(19) United States
(12) Patent Application Publication
(10) Pub. No.: US 2015/0056627 A1
(43) Pub. Date: Feb. 26, 2015

(54) APTAMERS

(71) Applicants: DuPont Nutrition Biosciences ApS, Copenhagen (DK); The University of Chester, Chester (GB)

(72) Inventors: Riikka Kärkkäinen, Chester (GB); Mette Ryun Drasbek, Sabro (DK); Niall W.G. Young, Tjele (DK); Graham A. Bonwick, Middlewich (GB); Christopher Smith, Rhualt (GB)

(21) Appl. No.: 14/355,351

(22) PCT Filed: Oct. 31, 2012
(86) PCT No.: PCT/GB2012/052702

§ 371 Date: Apr. 30, 2014

Related U.S. Application Data
(60) Provisional application No. 61/553,501, filed on Oct. 31, 2011.

(51) Int. Cl. G01N 33/569

(52) U.S. Cl. CPC G01N 33/56916
USPC 435/6.15; 536/23.1

(57) ABSTRACT

A nucleic acid aptamer comprising the nucleotide sequence shown herein as SEQ ID No. 5, 6, 7, 8, 9, 10, 1, 2, 3 or 4 or a fragment thereof or a sequence which is at least 80% identical therewith and use of nucleic aptamers to detect the presence of pathogenic bacteria in a sample, particularly in a complex matrix—such as a food system.

### FIELD OF THE INVENTION

The present invention relates to novel nucleic acid aptamers and their uses.

### BACKGROUND OF THE INVENTION

Aptamers are biomolecular ligands composed of nucleic acids. They can be selected to bind specifically to a range of target molecules such as proteins, bacterial cells, viruses and smaller molecular targets such as organic dyes. They can subsequently be exploited in a fashion analogous to more traditional biomolecules such as antibodies. Aptamers can be chemically synthesised. Therefore, in contrast to antibodies, no ethical issues are involved in aptamer production. The potential of aptamers and the need for development of new aptamers with specificity against pathogenic micro-organisms will be discussed.

Food can often be contaminated by a range of pathogenic micro-organisms. These contaminants, or products of, can spoil the food production or cause various illnesses ranging from the mildly uncomfortable to the life-threatening. Therefore, rapid detection of the pathogens is important for health and safety reasons.

Traditional detection methods, such as commonly used culture methods, are time consuming. Current principle methodologies for the rapid detection of food poisoning bacteria, such as immunoassays and the polymerase chain reaction (PCR), have significantly reduced the detection time compared to traditional culture methods. A commonly used antibody based method for pathogen detection is the enzyme-linked immunosorbent assay (ELISA) that makes use either of monoclonal or polyclonal antibodies. These are generally prepared in animals or in cell cultures derived from the tissues or organs of animals or humans (Bonwick & Smith, 2004; Karoonuthaisiri et al., 2009). Using animal derived material has ethical and moral considerations making the alternative ‘synthesised’ biomolecules more attractive.

In 1990, Tuerk & Gold and Ellington & Szostak first described specific nucleotide molecules that can be selected to bind to proteins. They called these high-affinity single-stranded DNA or RNA molecules, ‘aptamers’, a name derived from the Latin term aptus, ‘to fit’. Since their discovery, the techniques for isolating aptamers have been developed (Vivekananda & Kiel, 2003; Hamula et al., 2008; Cao et al., 2009) and aptamers have been targeted to bind to several different targets including proteins, bacterial cells (Hamula et al., 2008), viruses (Symensma et al., 1996), prions (Takemura et al., 2006) and smaller molecular targets such as organic dyes (Ellington & Szostak, 1990).

Aptamers can be selected in vitro through the technique described by Tuerk & Gold (1990) as the systematic evolution of ligands by exponential enrichment (SELEX). Once aptamers have been identified they can be inexpensively produced either synthetically or enzymatically (Pendergrast et al., 2005) and no animals or animal derived cells are needed for their production. This has the potential to lead to cheaper production costs when compared to antibodies. Aptamers can also be stored as a lyophilised powder at room temperature for more than one year (Pendergrast et al., 2005) and they can recover their native active conformation after denaturation. They are also more stable at higher temperatures than antibodies, which only normally function under physiological conditions (Tombelli et al., 2007).

Despite their potential, aptamers have not yet been accepted and routinely used particularly for the analysis of complex matrices such as food (Karkkainen et al., 2011 International Journal of Food Science and Technology 46(3), 445-454).

Therefore an object of the present invention is to provide novel aptamers for use in analysing complex matrices, such as food and/or for use in detecting pathogenic microorganisms in a sample (preferably in a complex matrix, such as food).

### BRIEF DESCRIPTION OF THE DRAWINGS

FIG. 1 shows the predicted secondary structure for aptamer 6AptK12—the sequences are given in table 2.1 in Example 1;

FIG. 2 shows FAM-labelled aptamers binding to E. coli K12 cells. The fluorescence (495 nm, Em 520) was measured by a plate reader in triplicate (n=3). The values are presented as means±s.d. Fluorescence has been corrected for background.

FIG. 3 shows FAM-labelled aptamers 1AptK12, 2AptK12, 4AptK12 and 6AptK12 (50 pmol) binding to the surface of E. coli K12. Pictures were taken with a fluorescence microscope with 60× magnification with a green (495 nm) light.

FIG. 4 shows FAM-labelled aptamers 1AptK12, 2AptK12, 4AptK12 and 6AptK12 binding to E. coli K12 extracted from yoghurt. The fluorescence (495 nm, Em 520) was measured by a plate reader (n=3). The values are presented as means±s.d. (F=36.75, P-value 3.7×10⁻³).

FIG. 5 shows FAM-labelled aptamers binding to E. coli O157 497 cells. The fluorescence (495 nm, Em 520) was measured by a plate reader in triplicate (n=3). The values are presented as means±s.d. Fluorescence has been corrected for background.

FIG. 6 shows FAM-labelled aptamers binding to live S. typhimurium 223 cells. Fluorescence (495 nm, Em 520) was measured by a plate reader in triplicate (n=3). The values are presented as means±s.d. Fluorescence has been corrected for background.

FIG. 7 shows FAM-labelled aptamers (2Apt223, 3Apt223 and 5Apt223) binding to live Salmonella enteritidis, Salmonella typhimurium, E. coli K12 and Listeria plantarum. Fluorescence (495 nm, Em 520) was measured by a plate reader in triplicate (n=3). The values are presented as means±s.d. Fluorescence has been corrected for background.

FIG. 8 shows an illustration of enzyme linked technique for detection of bound aptamers. Biotin labelled aptamer bound to target cell wall and peroxidase (Px) labelled streptavidin (SA) has bound to biotin. The colour change appears when ABTS substrate reacts with peroxidase.

FIG. 9 shows a schematic of Aptamer cloning. 1. PCR amplification of the aptamer pools. 2. Ligation of the aptamers (insert) into linearised plasmid vector. 3. Transformation of the vector with an aptamer insert into the competent bacterial cells. 4. Growth of bacteria and enrichment of the cloned plasmid during the normal bacterial growth.

FIG. 10 shows pGEM®-T Easy Vector map and sequence reference points (Promega Technical manual).

FIG. 11 shows sequence and multi-cloning site of pGEM®-T Easy Vector (adapted from Promega Technical manual).

FIG. 12 shows sequence and multi-cloning site of pGEM®-T Easy Vector (adapted from Promega Technical manual) with sequencing primer sites (in red).

FIG. 13 shows 2% Agarose gel with the DNA library and non-specific products. Lane M on the gel contains PCR Sizer 100 bp DNA Ladder, lane 0 is a PCR control and in lane 1 is a PCR amplified DNA library with 2.5 pmol template DNA.

FIG. 14 shows homo- and heterodimers that can be formed between the primers PR1 and PR2. The oligonucleotides were analysed with an Oligoanalyzer.

FIG. 15 shows 2% Agarose gel with DNA library. Lane M on the gel contains PCR MiniSizer 50 bp DNA Ladder, lane 0 is a PCR control sample (no template DNA added), lanes 1, 2, and 3 are DNA library with 0.1 pmol template DNA and lanes 4, 5, and 6 are DNA library with 0.5 pmol template DNA.

FIG. 16 shows agarose gel (2%) with aptamer pool 1 (a), 2 (b), 3 (c), and 4 (d) with two replicates. Lane M on the gel pictures contain PCR Sizer 100 bp DNA Ladder, lane 0 is the PCR control sample. The bacterial control samples are in lane 3 and DNA controls in lane 4.

FIG. 17 shows agarose gels (2%) with aptamer pool 5 (lanes 1 and 2) before (a) and after counter selection with L. bulgaricus (b). Lane M contains PCR Sizer 100 bp DNA Ladder and lanes 0 is the PCR control sample. On gel A the bacterial control sample is in lane 3 and DNA control in lane 4. On gel C, template control is in lane 1.

FIG. 18 shows agarose gel (2%) with aptamer pool 6 (Ap6), and 7 (Ap7). Lane M on gel A contains the PCR Sizer 100 bp DNA Ladder and on gel B PCR MiniSizer 50 bp DNA Ladder, PCR control samples is in lane 0. The bacterial control samples are in lane 3 and DNA controls in lane 4. On gel B two aptamer pools were produced in replicates (lanes 1.1Ap7, 1.2Ap7, 2.1Ap7 and 2.2Ap7).

FIG. 19 shows agarose gel (2%) with aptamer pool 8 before (a) and after (b) counter selection with B. subtilis and S. typhimurium. Lane M on the gels contains PCR MiniSizer 50 bp DNA Ladder and lane 0 shows the PCR control sample (no template DNA added). On gel A aptamer pool 8 is in lane 1.1, 1.2, 2.1, and 2.2. On gel B aptamer pool 8 after the counter selection is in lanes 1 and 2.

FIG. 20 polyacrylamide gel (10%) with aptamer pool 9. Lane M on the gel contains PCR MiniSizer 50 bp DNA Ladder, lane 0 is PCR control sample. On lane 1 and 2 is aptamer pool 9. The bacterial control sample is on lane 3 and DNA control on lane 4. The PCR was repeated 20 times.

FIG. 21 shows agarose gel (2%) with biotin-labelled aptamer pool 9. Lane M is PCR MiniSizer 50 bp DNA Ladder, lane 0 is a PCR control sample and lanes 1-12 are the PCR amplified aptamer biotin-labelled aptamer pool 9.

FIG. 22 shows agarose gel (2%) with FAM-labelled E. coli K12 binding aptamer pool 9. Lane M on the gel contains PCR MiniSizer 50 bp DNA Ladder, lane 0 is a PCR control sample (no template DNA added) and FAM-labelled aptamers with an aptamer pool 9 are in lanes 1-7.

FIG. 23 shows FAM-labelled aptamer pool 9 binding to E. coli K12 cells. The fluorescence (495 nm, Em 520) was measured by a plate reader in triplicate (n=3). The values are presented as means±s.d. Fluorescence has been corrected for background. (F=71.85, p=3.98×10⁻⁶).

FIG. 24 shows FAM-labelled aptamer pool 9 binding to the surface of E. coli K12. Images were taken with a fluorescence microscope with 60× magnification with a green light (495 nm) and visible light. No aptamers were added to the control sample (0 pmol) while 10 pmol and 50 pmol aptamers were added to the samples.

FIG. 25 shows FAM-labelled aptamer pool binding to the surface of E. coli K12. Image was taken with a fluorescence microscope with 60× magnification with a green light (495 nm). The long structure circled was a typical finding in fluorescence images that might indicate aptamers binding to the bacterial cells in the division stage of their life cycle.

FIG. 26 shows a number of fluorescent labelled bacteria. Two concentrations (20 pmol and 50 pmol) of aptamers were incubated with E. coli K12 and the fluorescence images were taken from six random fields (n=6). The green bacterial cells were counted from the images and the values presented are means±s.d. (F=34.8, p=1.5×10⁻⁴).

FIG. 27 shows optimal binding time of the aptamers. FAM-labelled aptamer pool 9 (˜6 pmol) was incubated with bacterial cells and the fluorescence (495 nm, Em 520) was measured by a plate reader after 0, 15, 30, 45, 60 and 75 min in triplicate (n=3). The values are presented as means±s.d. Fluorescence has been corrected for background. (F=30.4, p=2.04×10⁻⁶).

FIG. 28 shows fluorescence of non-binding aptamers after the first wash. FAM-labelled aptamer pool 9 (˜6 pmol) was incubated with E. coli K12. Fluorescence (495 nm, Em 520) was measured after 0, 15, 30, 45, 60 and 75 min in triplicate (n=3). The values are presented as means±s.d. Fluorescence has been corrected for background.

FIG. 29 shows fluorescence of non-binding aptamers after the 2nd and 3rd wash. FAM-labelled aptamer pool 9 (˜6 pmol) was incubated with E. coli K12. Fluorescence (495 nm, Em 520) was measured after 0, 15, 30, 45, 60 and 75 min in triplicate (n=3). The values are presented as means±s.d. Fluorescence has been corrected for background.

FIG. 30 shows FAM-labelled aptamer pools 3, 5, 7 and 9 binding to E. coli K12 bacterial cells. The fluorescence (495 nm, Em 520) measured by the plate reader (n=1). Fluorescence values were corrected for background.

FIG. 31 shows specificity of E. coli K12 specific aptamer pool 9. FAM-labelled aptamers were incubated with E. coli K12 (positive control), E. coli B, B. subtilis and S. aureus. The fluorescence (495 nm, Em 520) was measured by a plate reader (n=1). Fluorescence has been corrected for background.

FIG. 32 shows specificity of the E. coli K12 binding aptamers. Aptamers were incubated with E. coli K12 (positive control), E. coli B and the images were taken with a fluorescence microscope with 60× magnification with a green light (495 nm) and visible light. No aptamers were added to the control sample (0 pmol) while 20 pmol aptamers were added to the samples.

FIG. 33 shows specificity of the E. coli K12 binding aptamers. Aptamers were incubated with B. subtilis and S. aureus and the images were taken with a fluorescence microscope with 60× magnification with a green light (495 nm) and visible light. No aptamers were added to the control sample (0 pmol) while 20 pmol aptamers were added to the samples.

FIG. 34 shows the number of fluorescent labelled bacteria. Aptamers (20 pmol) were incubated with E. coli K12, E. coli B and S. aureus and the fluorescence images were taken from five random fields (n=5). The green bacterial cells were counted from the images. The values presented are means±s.d. (F=75.8, p=1.55×10⁻⁷).

FIG. 35 shows detection of E. coli K12 from a mixture of bacterial cells. FAM-labelled aptamer pool nine was incubated with a mixture of E. coli K12, E. coli B and S. aureus (Mix) and with each strain separately. The fluorescence (495 nm, Em 520) was measured by a plate reader (n=1). Fluorescence has been corrected for background.

FIG. 36 show specificity of E. coli K12 specific aptamer pool 9. FAM-labelled aptamers were incubated with E. coli K12 (positive control), E. coli B, S. aureus and L. acidophilus. The fluorescence (495 nm, Em 520) was measured by a plate reader (n=1). Fluorescence has been corrected for background.

FIG. 37 shows biotin labelled aptamer pool 9 bound to E. coli K12. FluoSpheres were bound to biotin and the images were taken with a fluorescence microscope with 100× magnification. Biotin labelled aptamers incubated with E. coli K12 (+) and no aptamers added to the negative (−) control sample. Normal sized images are on left hand side column and zoomed images on right hand side. An example where FluoSpheres are binding to bacterial cell is circled.

FIG. 38 shows FAM-labelled aptamer pool 9 bound to E. coli K12 followed by Live/Dead BacLight straining. The images were taken with a fluorescence microscope with 100× magnification.

FIG. 39 shows biotin labelled aptamer pool 9 bound to E. coli K12. FluoSpheres were bound to biotin followed by LIVE/DEAD BacLight staining. The images were taken with a fluorescence microscope with 100× magnification. Biotin labelled aptamers incubated with E. coli K12 (+) and no aptamers added to the negative (−) control sample. Green colour indicates the cells are alive whilst red colour indicates the cells are dead.

FIG. 40 shows agarose gel image of the PCR Spermix HiFi analysis for positive colonies. Lane M on the gel contains PCR MiniSizer 50 bp DNA Ladder and on lane 0 is a PCR control sample. On lane CI1-CI8 are the cloned colonies. Sample c is the plasmid control sample.

FIG. 41 shows agarose gel image of the restriction (EcoRI) products. Lane M on the gel contains PCR Sizer 100 bp DNA Ladder. Restriction products for cloned plasmids are in lanes CI1-CI8.

FIG. 42 shows agarose gel images for FAM-labelled cloned aptamers CI1 and CI2. Lane M on the gel contains PCR MiniSizer 50 bp DNA Ladder and lane 0 is a PCR control sample (no template DNA added).

FIG. 43 shows FAM-labelled cloned aptamers CI1 and CI2 binding to E. coli K12 cells. The fluorescence (495 nm, Em 520) was measured by a plate reader (n=1). Fluorescence has been corrected for background.

FIG. 44 shows predicted aptamer secondary structures (OligoAnalyzer 3.1, UNAFold) at 25° C. (NaCl 100 mM, MgCl 1 mM). The two strongest secondary structures for E. coli K12 binding nucleotide sequence (2CI-AptK12). Aptamers 1AptK12 and 2AptK12 has been created by cutting off (*) the possible binding sites from the 100 nt sequence. Isolated sequences are circled. Dots are representing the base-pair interactions.

FIG. 45 shows predicted aptamer secondary structures (OligoAnalyzer 3.1, UNAFold) at 25° C. (NaCl 100 mM, MgCl 1 mM). The two strongest secondary structures for E. coli K12 binding nucleotide sequence (3CI-AptK12). Aptamers 3AptK12 and 4AptK12 has been created by cutting off (*) the possible binding sites from the 100 nt sequence. Isolated sequences are circled. Dots are representing the base-pair interactions.

FIG. 46 shows predicted aptamer secondary structures (OligoAnalyzer 3.1, UNAFold) at 25° C. (NaCl 100 mM, MgCl 1 mM). The two strongest secondary structures for E. coli K12 binding nucleotide sequence (4CI-AptK12). Aptamers 5AptK12 and 6AptK12 have been created by cutting off (*) the possible binding sites from the 100 nt sequence. Isolated sequences are circled. Dots represent the base-pair interactions.

FIG. 47 shows FAM-labelled aptamers binding to E. coli K12 cells. The fluorescence (495 nm, Em 520) was measured by a plate reader in triplicate (n=3). The values are presented as means±s.d. Fluorescence has been corrected for background. This is a duplicate of FIG. 2.

FIG. 48 shows FAM-labelled aptamers 1AptK12, 2AptK12, 4AptK12 and 6AptK12 (50 pmol) binding to the surface of E. coli K12. Images were taken with a fluorescence microscope with 60× magnification with a green (495 nm) and visible light.

FIG. 49 shows a mixture of FAM-labelled aptamers (1AptK12, 2AptK12, 4AptK12 and 6AptK12) binding to E. coli K12, E. coli B and S. aureus. The fluorescence (495 nm, Em 520) was measured by a plate reader in triplicate (n=3). The values are presented as means±s.d. Fluorescence has been corrected for background. (F=626.1, p=1.08×10⁻⁷).

FIG. 50 shows aptamer specificity. A mixture of FAM-labelled aptamers (50 pmol) 1AptK12, 2AptK12, 4AptK12 and 6AptK12 incubated with the positive control E. coli K12, and the test strains E. coli B and S. aureus. Negative control samples (0 pmol) were performed with no aptamers. Images were taken with a fluorescence microscope with 60× magnification with a green light (495 nm, Em 520) and visible light.

FIG. 51 shows FAM-labelled aptamers 1AptK12, 2AptK12, 4AptK12 and 6AptK12 (20 pmol) binding to E. coli K12, E. coli B and S. aureus. The fluorescence (495 nm, Em 520) was measured by a plate reader in triplicate (n=3). The values are presented as means±s.d. Fluorescence has been corrected for background.

FIG. 52 shows FAM-labelled aptamer pool 9 binding to E. coli K12 in tap water. The fluorescence (495 nm, Em 520) was measured by a plate reader (n=1). The values are corrected for background.

FIG. 53 shows FAM-labelled aptamer pool 9 binding to E. coli K12 extracted from yoghurt. The fluorescence (495 nm, Em 520) was measured by a plate reader in triplicate (n=3). The values are presented as means±s.d. The samples are corrected for background. (F=34.27, p=4.2×10⁻³)

FIG. 54 shows FAM-labelled aptamer pool 9 binding to E. coli K12 extracted from yoghurt. The fluorescence (495 nm, Em 520) was measured by a plate reader (n=2). The values are presented as means±s.d. The vales are corrected for background. (F=18.6, p=0.05).

FIG. 55 shows FAM-labelled aptamers 1AptK12, 2AptK12, 4AptK12 and 6AptK12 binding to E. coli K12 extracted from natural probiotic yoghurt. The fluorescence (495 nm, Em 520) was measured by a plate reader (n=3). The values are presented as means±s.d. (F=36.75, p=3.7×10⁻³).

FIG. 56 shows 2% Agarose gel with aptamer pool for E. coli 496 (lanes 1 and 2) and E. coli O157 497 (lanes 34). The bacterial control samples are in lanes 5 and 6 and the DNA control is in lane 7. The M on the gel is a PCR Sizer 100 bp DNA Ladder and the 0 is the PCR control.

FIG. 57 shows aptamer pool 5 (a and b), 6 (c) and 7 (d) for L. innocua 17 (black boxes), L. monocytogenes 489 (grey boxes) and L. monocytogenes 490 (white boxes) on 2% agarose gel. The bacterial control samples are in lanes 7, 8 and 9 (c) and in lane 7 (d). The DNA control is in lane 10 (c) and 8 (d). The M on the gel is a PCR Sizer 100 bp DNA Ladder and the 0 is the PCR control.

FIG. 58 shows aptamer pool 8 (a) and 9 (b) for L. monocytogenes 490 (white boxes) on 2% agarose gel. Lane M on the gel is a PCR Sizer 100 bp DNA Ladder and lane 0 is the PCR control. Bacterial control is in lane 3 and the DNA control in lane 4 (a).

FIG. 59 shows aptamer pools 1 (a), 2 (b), 3 (c) and 4 (d) for S. typhimurium 223 (black boxes) and S. enteritidis 1152 (white boxes) on agarose gel (2%). Lane M is a PCR Sizer 100 bp DNA Ladder and lane 0 is the PCR control. Bacterial control sample is in lane 3 for 223 and in lane 6 for 1152. The DNA control sample is in lane 7.

FIG. 60 shows S. typhimurium 223 (black box) and S. enteritidis 1152 (white box) aptamer pool 5 on agarose gel (2%). The M on the gel is a PCR Sizer 100 bp DNA Ladder and in lane 0 is the PCR control. Bacterial control samples are in lanes 3 and 6 and the DNA control sample is in lane 7.

FIG. 61 shows aptamer pools 6 (a), 7 (b), 8 (c) and 9 (d) for S. typhimurium 223 (black boxes) and S. enteritidis 1152 (white boxes) on agarose gel (2%). Lane M on the gels is a PCR Sizer 100 bp DNA Ladder and in lane 0 is the PCR control. Bacterial control samples are in lanes 1 and 2 on gel A and in lane 5 on gel C. The DNA control samples are in lane 3 (a) and in lane 6 on gel c.

FIG. 62 shows 2% Agarose gel with the PCR Spermix HiFi analysis for positive colonies. Lane M on the gel contains PCR MiniSizer 50 bp DNA Ladder. In lanes CI1s-CI6s are the cloned colonies for S. typhimurium aptamers and in lanes CI1e-CI6e are the cloned colonies for E. coli aptamers. In lane 0 is a PCR control sample and N is negative control.

FIG. 63 shows 2% Agarose gel with the purified plasmid vectors. Lane M on the gel contains FullRanger 100 bp DNA Ladder. In lanes CI1s-CI6s are the positive clones for S. typhimurium aptamers and in lanes CI2e-CI6e are the positive clones for E. coli aptamers.

FIG. 64 shows predicted aptamer secondary structures (OligoAnalyzer 3.1, UNAFold) at 25° C. (NaCl 100 mM, MgCl 1 mM). The two strongest secondary structures for E. coli O157 497 binding nucleotide sequence (3CI-Apt497). Aptamer 1Apt497 has been created by cutting off (*) the possible binding sites from the 100 b sequence. Isolated sequences are circled. Blue and red dots are representing the base-pair interactions.

FIG. 65 shows predicted aptamer secondary structures (OligoAnalyzer 3.1, UNAFold) at 25° C. (NaCl 100 mM, MgCl 1 mM). The two strongest secondary structures for E. coli O157 497 binding nucleotide sequence (4CI-Apt497). Aptamers 2Apt497 and 3Apt223 has been created by cutting off (*) the possible binding sites from the 100 b sequence. Isolated sequences are circled. Blue and red dots represent the base-pair interactions.

FIG. 66 shows predicted aptamer secondary structures (OligoAnalyzer 3.1, UNAFold) at 25° C. (NaCl 100 mM, MgCl 1 mM). The two strongest secondary structures for E. coli O157 497 binding nucleotide sequence (5CI-Apt497). Aptamers 4Apt497 and 5Apt497 have been created by cutting off (*) the possible binding sites from the 100 b sequence. Isolated sequences are circled. Blue and red dots represent the base-pair interactions.

FIG. 67 shows predicted aptamer secondary structures (OligoAnalyzer 3.1, UNAFold) at 25° C. (NaCl 100 mM, MgCl 1 mM). The two strongest secondary structures for S. typhimurium 223 binding nucleotide sequence (1 CI-Apt223). Aptamers 1Apt223 and 2Apt223 has been created by cutting off (*) the possible binding sites from the 100 b sequence. Isolated sequences are circled. Blue and red dots represent the base-pair interactions.

FIG. 68 shows predicted aptamer secondary structures (OligoAnalyzer 3.1, UNAFold) at 25° C. (NaCl 100 mM, MgCl 1 mM). The two strongest secondary structures for S. typhimurium 223 binding nucleotide sequence (2CI-Apt223). Aptamer 3Apt223 has been created by cutting off (*) the possible binding site from the 100 b sequence. Isolated sequences are circled. Blue and red dots represent the base-pair interactions.

FIG. 69 shows predicted aptamer secondary structures (OligoAnalyzer 3.1, UNAFold) at 25° C. (NaCl 100 mM, MgCl 1 mM). The two strongest secondary structures for S. typhimurium 223 binding nucleotide sequence (3CI-Apt223). Aptamers 4Apt223 and 5Apt223 has been created by cutting off (*) the possible binding sites from the 100 b sequence. Isolated sequences are circled. Blue and red dots represent the base-pair interactions.

FIG. 70 shows predicted aptamer secondary structures (OligoAnalyzer 3.1, UNAFold) at 25° C. (NaCl 100 mM, MgCl 1 mM). The two strongest secondary structures for S. typhimurium 223 binding nucleotide sequence (4CI-Apt223). Aptamer 6Apt223 has been created by cutting off (*) the possible binding sites from the 100 b sequence. Isolated sequences are circled. Blue and red dots represent the base-pair interactions.

FIG. 71 shows FAM-labelled aptamers binding to E. coli O157 497 cells. The fluorescence (495 nm, Em 520) was measured by a plate reader in triplicate (n=3). The values are presented as means±s.d. Fluorescence has been corrected for background.

FIG. 72 shows microscopy images of FAM-labelled aptamers 1Apt497, 2Apt497 and 4AptK12 (20 pmol) binding to the surface of E. coli O157 497. Images were taken with a fluorescence microscope with 100× magnification with a green (495 nm) and visible light.

FIG. 73 shows FAM-labelled aptamers 1Apt497, 2Apt497, 4Apt497 and 4AptK12 binding to live E. coli K12 cells. Fluorescence (495 nm, Em 520) was measured by a plate reader in triplicate (n=3). The values are presented as means±s.d. Fluorescence has been corrected for background.

FIG. 74 shows microscopy images of FAM-labelled E. coli O157 aptamers 1Apt497, 2Apt497 and 4Apt497 (20 pmol) binding to the surface of E. coli K12. Images were taken with a fluorescence microscope with 60× magnification with a green (495 nm) and visible light.

FIG. 75 FAM-labelled aptamers binding to live S. typhimurium 223 cells. Fluorescence (495 nm, Em 520) was measured by a plate reader in triplicate (n=3). The values are presented as means±s.d. Fluorescence has been corrected for background.

FIG. 76 shows microscopy images of FAM-labelled aptamers 2Apt223, 3Apt223 and 5Apt223 (20 pmol) binding to the surface of S. typhimurium 223. Images were taken with a fluorescence microscope with 100× magnification with a green (495 nm) and visible light.

FIG. 77 shows specificity of S. typhimurium 223 aptamers. Fluorescence (495 nm, Em 520) was measured by a plate reader in triplicate (n=3). The values are presented as means±s.d. Fluorescence has been corrected for background.

FIG. 78 shows microscopy images showing the binding of the FAM-labelled aptamer 3Apt223 to S. typhimurium and S. enteritidis. Images were taken with a fluorescence microscope with 100× magnification with a green (495 nm) and visible light.

FIG. 79 shows microscopy images showing the binding of the FAM-labelled aptamer 3Apt223 to E. coli K12 and L. plantarum. Images were taken with a fluorescence microscope with 100× magnification with a green (495 nm) and visible light.

### SUMMARY OF THE INVENTION

A seminal finding of the present invention is the development of novel aptamers and the fact that these aptamers have high specificity for pathogenic microorganisms (particularly pathogenic bacteria).

For the first time the inventors have shown that the aptamers of the present invention have specificity for live pathogenic microorganisms (particularly pathogenic bacteria).

In addition the inventors have demonstrated the feasibility of using the aptamers of the present invention in complex matrices (e.g. real food systems) to target and detect specific microorganisms (e.g. microbial food contaminants).

The inventors have developed the aptamers of the present invention using a novel selection method using centrifugation (see section 2.3.6 below).

Based on these findings we provide a new rapid detection method for microorganisms, e.g. food spoilage microorganism, or pathogenic microorganisms in a sample, preferably in a complex matrix.

### STATEMENTS OF THE INVENTION

According to a first aspect the present invention provides a nucleic acid aptamer which specifically binds a pathogenic microorganism, preferably a pathogenic bacterium.

According to a further aspect the present invention provides a nucleic acid aptamer comprising the nucleotide sequence shown herein as SEQ ID No. 5, 6, 7, 8, 9, 10, 1, 2, 3 or 4, or a fragment thereof, or a sequence which is at least 80% identical therewith, or a sequence which hybridises under stringent conditions therewith.

In another aspect the present invention provides a kit comprising at least one nucleic acid aptamer according to any one of the preceding claims together with instructions on how to use the at least one nucleic acid aptamer.

The present invention yet further provides a device (preferably a hand-held or portable device) comprising at least one of the nucleic aptamers of the present invention or capable of detecting at least one of the nucleic aptamers of the present invention.

The present invention further provides a microarray or biosensor comprising at least one of the nucleic acid aptamers of the present invention.

A further aspect of the present invention provides a method of detecting a microorganism in a sample comprising admixing a nucleic acid aptamer according to the present invention with the sample and identifying the presence of a bound aptamer.

In another aspect of the present invention there is provided use of a nucleic acid aptamer according to the present invention for detecting a microorganism in a sample.

In a yet further aspect of the present invention there is provided a method of selecting aptamers, wherein the aptamer is selected on its ability to bind (e.g. specifically bind) to live bacterial cells, preferably live pathogenic bacterial cells, which method comprises the steps of exposing an aptamer to live bacterial cells (preferably live pathogenic bacterial cells) and selecting an aptamer which binds (e.g. specifically) binds to said live bacterial cells, optionally said method further comprises a washing and centrifuging (e.g. at 3500-4000 g for 5 min at 4° C.) step. In one embodiment, said method of selecting aptamers comprises two washing and centrifuging (e.g. at 3500-4000 g for 5 min at 4° C.) steps. When the method comprises two washing and centrifuging steps one occurs before aptamer binding and the other one occurs after aptamer binding.

### DETAILED DISCLOSURE OF THE PREFERRED EMBODIMENTS OF THE INVENTION

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Other definitions of terms may appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and reference to “the feed̂” includes reference to one or more feedŝ and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

Preferably the nucleic acid aptamer according to the present invention comprises a nucleotide sequence shown herein as SEQ ID No. 5, 6, 7, 8, 9 or 10, or a fragment thereof, or a sequence that hybridises under stringent conditions thereto.

Suitably the nucleic acid aptamer according to the present invention comprises at least 10, preferably at least 20, preferably at least 30, more preferably at least 40 nucleotides.

Suitably the nucleic acid aptamer according to the present invention comprises at most 70 nucleotides in length, preferably at most about 60 nucleotides in length.

Suitably the nucleic acid aptamer according to the present invention comprises in the region of 40 to 60 nucleotides, preferably 42-59 nucleotides.

In one embodiment the nucleic acid aptamer according to the present invention comprises about 50 nucleotides.

Preferably the nucleic acid aptamer has specificity against a live pathogenic bacterium.

The term “specificity” as used herein means that the aptamer is selectively reactive with live pathogenic bacteria compared with either dead pathogenic bacteria or live non-pathogenic bacteria.

In some embodiments aptamers have specificity for a particular genera, species or strain of pathogenic bacteria. By way of example only the aptamer may be selective for Salmonella spp. (such as Salmonella typhimurium, e.g. Salmonella typhimurium 233, Salmonella enteritidis), Escherichia coli spp. (such as E. coli O157) or Listeria spp. In this regard the term “specificity” would mean that the aptamer preferentially selects that genera, species or strain over any other genera, species or strain and/or that there is no or insignificant cross-reactivity with other genera, species or strains.

Preferably the nucleic acid aptamers are synthetic.

The aptamers according to the present invention may be used in a fashion analogous to antibodies. Like antibodies the aptamers provide target binding specificity.

The aptamers may be modified by addition of one or more reporter labels (or detectable labels).

In some embodiments the label may be attached to either the 5′ or 3′ end of the aptamer. In a preferred embodiment the label may be attached to the 5′-end of the aptamer.

The skilled person will be aware of techniques for attaching labels to nucleic acid strands. Any one of these methods may be utilised in the present invention to attach a detectable label to the nucleic acid aptamers.

In some embodiments the aptamer may be synthesized by Eurofins MWG Operon, Modified DNA oligos (Oligos a la carte) and FAM (6-carboxyfluroescein) may be attached to the 5′-end.

In one embodiment the nucleic acid aptamer comprise a detectable label. The detectable label may be attached directly or indirectly to the nucleic acid aptamer. If the label is indirectly attached to the nucleic acid aptamer this may be by any mechanism known to one of skill in the art, such as using biotin and streptavidin.

Suitably, the aptamer may comprise a reporter label, such as a fluorescent dye or an enzyme.

Suitably, the aptamer may comprise a fluorescent label.

In some embodiments, the reporter label may comprise one or more parameter(s) for detection.

The parameters may be for example the size of the label and/or the optical properties of the label,

In some embodiments, the optical properties are selected from the group consisting of: light reflectivity, colour, the fluorescence emission wavelength(s) and the fluorescence emission intensity.

In some embodiments, the properties of each label may be measured using microscopy.

In some embodiments, the microscopy method is selected from the group consisting of bright field microscopy, phase-contrast microscopy, oblique illumination microscopy, dark field microscopy, differential interference contrast microscopy, reflection contrast microscopy, contrast microscopy, polarizing microscopy, interference microscopy and fluorescence microscopy.

In one embodiment UV illumination may be used to detect labelled (e.g. fluorescently labelled) aptamers. The detected aptamers may be bound direct to a target (e.g. a microorganism, such as a pathogenic microorganism).

In some embodiments, the fluorophore is selected from the group consisting of a fluorophore that emits a blue, green, near red or far red fluorescence.

In some embodiments, where two or more fluorophores are used, the fluorophores do not quench each other.

In some embodiments, the sizes are selected from the group consisting of about 1.9 μm, about 4.4 μm, about 5.4 μm, about 5.8 μm, about 7.4 μm, about 9.7 μm and about 9.8 μm

In some embodiments, the fluorophore is selected from the group consisting of UV2, Starfire Red and TRITC.

In one embodiment the aptamer may comprise biotin (or be modified to include biotin) for binding with streptavidin.

In another embodiment the aptamer may be pegylated, for example to minimise degradation if used therapeutically in vivo.

The aptamer(s) of the present invention may be immobilised on (e.g. bound or adhered to) a substrate or carrier, e.g. a microcarrier.

The aptamer(s) of the present invention may be immobilised on a magnetic bead, or microbead.

In some embodiments, the microcarrier is a porous or a solid microcarrier.

In some embodiments, the porous microcarrier is selected from the group consisting of Cytopore microcarrier (e.g. a Cytopore 1 microcarrier or a Cytopore 2 microcarrier), a Cultispher microcarrier, a Cultispher-G microcarrier, a Cultispher-GL microcarrier and a Cultispher-S microcarrier, an Informatrix microcarrier, a Microsphere microcarrier, a Siran microcarrier, and a Microporous MC microcarrier.

In some embodiments, the solid microcarrier is selected from the group consisting of a Cytodex microcarrier (eg. a Cytodex 1, Cytodex 2 or Cytodex 3 microcarrier) a Biosilon microcarrier, a Bioglass microcarrier, a FACT III microcarrier or a DE 52/53 microcarrier.

By way of example the aptamer(s) may be used in a device, a microarray, a biosensor, a rapid detection test such as a lateral flow assay (dipstick) or a microplate based assay (e.g. analogous to ELISA).

The present invention further provides a microarray or biosensor comprising at least one of the nucleic acid aptamers of the present invention.

In some embodiments the microarray or biosensor may comprise more than one aptamer (optionally in combination with one or more antibodies) wherein at least one of the aptamers is an aptamer in accordance with the present invention.

In some embodiments the device in accordance with the present invention may be a lateral flow device. A lateral flow assay may also be known as a Lateral Flow Immunochromatographic Assay. A lateral flow device is intended to detect the presence (or absence) of a target analyte in a sample (e.g. complex matrix). Most commonly these tests are used for medical diagnostics either for home testing, point of care testing or laboratory use. The later flow device may be in a dipstick format. A lateral flow test is a form of assay in which the test sample flows along a solid substrate via capilliary action. After the sample is applied to the test it encounters a coloured reagent which mixes with the sample and transits the substrate encountering lines or zones which have been pretreated with the aptamer. Depending upon the analytes present in the sample the coloured reagent can become bound at the test line or zone. Thus the lateral flow device may give rise to a coloured band or spot.

In some embodiments the device in accordance with the present invention may be a microplate. The term microplate as used herein may also be referred to as a microtitre plate or microwell plate. The microplate may be a flat plate with multiple “wells” used as small test tubes.

The microplate may have 6, 12, 24, 48, 96, 384 or even 1536 sample wells arranged in a 2:3 rectangular matrix.

In one embodiment the microrarray or biosensor may comprise at least one nucleic acid aptamers of the present invention bound to a microcarrier.

The aptamer(s) of the present invention may be used in combination with an antibody (e.g. a target specific antibody) to produce a hybrid assay.

In one embodiment the device according the present invention is one capable of detecting colorimetric or fluorescence signal comprising:
i) a sample holder;
ii) an excitation light generating source including but not limited to a light emitting diode, a tungsten light, a halogen light or laser;
iii) a means to detect the emitted signal including but not limited to a photo diode or a photomultiplier tube.

In one embodiment the aptamers may be used in the selective purification and/or extraction of target molecules (e.g. microorganisms) from mixtures. This can be useful in pre-concentration steps. In some embodiments the aptamer(s) may be immobilised on a carrier (such as magnetic beads).

In one embodiment at least the nucleic acid aptamer according to the present invention may be supplied in a kit.

The kit according to the present invention may be a rapid detection test kit.

The kit may for example comprise i) at least one (such as 2, 3 or 4) labelled nucleic acid aptamer(s) according to the present invention and ii) instructions on how to use the aptamer(s).

In one embodiment the kit may comprise i) at least one fluorescently labelled nucleic acid aptamer according the present invention.

The kit of the present invention may further comprise a microcarrier. The microcarrier in the kit may be in a separate container to the nucleic acid aptamer(s). Alternatively, the kit may comprise nucleic acid aptamer(s) bound or adhered to a carrier, e.g. microcarrier.

The kit of the present invention may further comprise one or more antibodies, e.g. a target specific antibody.

In one embodiment the kit may comprise i) a biotin labelled aptamer and ii) an enzyme labelled streptavidin which enzyme reacts to provide a detectable label.

By way of example only the enzyme may be a peroxidase.

When the enzyme is a peroxidase the kit may optionally comprise iii) a 2,2′-azino-bis(3-ethylbenzthiazoline-6-sulphonic acid (ABTS) substrate and/or iv) hydrogen peroxide.

In one embodiment the kit according to the present invention comprises more than one (e.g. at least 2, such as at least 3) nucleic acid aptamers.

In one embodiment the kit according to the present invention may comprise:
I. A fluorescently labeled aptamer specific to a target of interest (e.g. a microorganism)—such as an aptamer in accordance with the present invention,
II. A binding reagent to enable binding of the aptamer to said target,
III. A washing reagent to remove unbound labeled aptamer, and
IV. A sample-resuspension agent or solid phase binding reagent to provide a carrying solution/binding phase to expose the aptamer labeled targets to a fluorescence detector.

The aptamers and/or kit may be used in a detection method (or detection assay) for detecting the presence of a microorganism in a sample.

Preferably the microorganism is a bacterium.

Preferably the microorganism is a pathogenic bacterium.

In one embodiment the pathogenic microorganism is selected from the group consisting of Salmonella spp. (such as Salmonella typhimurium, Salmonella enteritidis), Escherichia coli spp. (such as E. coli O157) or Listeria spp.

In one embodiment the aptamers of the present invention may be used to detect coliform bacteria in a food or water sample. Coliform bacteria are usually present in large numbers in the faeces of warm-blooded animals, and their detection in water and/or food samples can indicate contamination of the water or food. Coliform bacteria themselves may not cause serious illness, however their presence is used to indicate that other pathogenic organism of faecal original may be present. Therefore in one embodiment of the present invention the microorganism detected by aptamers may be a coliform bacterium, which may or may not be a pathogenic microorganism. Typical genera of coliform bacteria include Citrobacter, Enterobacter, Hafnia, Klebsiella, Serratia, Escherichia.

Preferably the sample is a complex matrix.

The aptamers according to the present invention may be used in a diverse range of diagnostic methods.

The sample (which is preferably a complex matrix) may be a food or feed sample, a beverage, a pharmaceutical sample, or a personal care sample.

The sample (which is preferably a complex matrix) may be a raw ingredient, a finished product or may be taken from the environment of manufacture or storage.

By “complex matrix” we mean a sample which comprises more than one component. The complex matrix may be a food or feed product, a beverage, a pharmaceutical product, or a personal care product.

By way of example only where the complex matrix is a food or feed—it may be meat or a meat product (e.g. raw or cooked meat product).

The term “admixing” as used herein means bringing the nucleic acid aptamer according to the present invention into contact with the sample. This may include bringing the aptamer into contact with the surface of a sample, for example the surface of meat or a meat product.

By way of example only where the complex matrix is a food or feed—it may be a dairy product or a composition used in the production of a dairy product, such as cheese or yoghurt.

By way of example only where the complex matrix is a food or feed—it may be a vegetable based food product.

By way of example only where the complex matrix is a food or feed—it may be a ready to eat food or a food ingredient.

By way of example only where the complex matrix is a food or feed—it may be a salad product, such as packaged vegetables, e.g. packaged lettuces.

By way of example only where the complex matrix is a food or feed—it may be infant formula.

The present invention may be used to detect for contamination of a food or feed with spoilage microorganism (e.g. spoilage bacteria) and/or pathogenic microorganisms (e.g. pathogenic bacteria).

In one embodiment the sample may be a personal care product such as an eye care product, such as contact lens solution.

In one embodiment the sample may be a beverage, such as beer or a sample taken during the brewing of beer. In other words the present invention may be used to detect beer spoilage bacteria.

In some embodiments the aptamers according to the present invention may be used in a manufacturing plant to detect for the presence of spoilage bacteria on or in equipment used therein.

It is envisaged that the aptamers of the present invention may be find use in public health applications. For example, the aptamers may be used to detect the presence of pathogenic bacteria in drinking water for instance. Thus in one embodiment the term “beverage” as used herein includes drinking water. The aptamers of the present invention may be used to detect faecal contamination of drinking water.

In some embodiments once the nucleic acid aptamer has been admixed with the sample, any unbound aptamer may be washed off before detecting the presence of bound aptamer.

Therefore the method of the present invention may comprise a further step of washing the admixture of aptamer and sample in order to remove any unbound aptamer.

The method of detecting the microorganism in a sample may comprise the steps of admixing the at least one aptamer with the sample, optionally centrifuging the sample and optionally washing the sample, followed by detecting the bound aptamer(s).

Suitably the aptamer may be admixed with the sample for at least 45 minutes before detecting the presence of bound aptamer. Preferably the sample and aptamer are admixed for about 1 hour before detection of the presence of bound aptamer.

Preferably the term “aptamer” as used herein means a single stranded DNA or RNA molecule, e.g. a high-affinity single stranded DNA or RNA molecule. The term “high-affinity” as used herein means that the aptamer readily (and preferably selectively) combines with the microorganism of interest.

In a preferred embodiment the aptamers of the present invention may be used for direct detection of a microorganism in a sample.

In an even more preferred embodiment the aptamers of the present invention may be used for direct detection of a microorganism in a complex matrix.

Preferably the method according to the present invention is an in vitro method.

The term “live” as used herein means that the microorganism, preferably bacterium, is capable of actively dividing or is actively dividing.

By way of example only the aptamers of the present invention may be used for direct detection of a pathogenic bacterium in a complex food matrix.

The term pathogenic as used herein means harmful to human or animal health. The pathogenic microorganism, e.g. pathogenic bacteria, may be one which causes food poisoning in humans.

The dosage of aptamer(s) used in accordance with the present invention may be determined by one of ordinary skill in the art. In any event, by way of guidance only it is envisaged that approximately 20 to 100 pmol of aptamer per 100 μm of sample would be sufficient for detection of the target microorganism(s). In one preferred embodiment 50 pmol of aptamer/100 μm of sample may be used.

### Advantages/Technical Effects

Aptamers can be used in a fashion analogous to antibodies because they exhibit target binding specificity. However aptamer production does not have moral and ethical issues associated with antibody production.

In addition, the cost of producing aptamers is significantly less than when producing antibodies.

One advantage of the present invention is that the method of detecting a microorganism in a sample in accordance with the present invention does not require enrichment of the microorganism (e.g. culturing of the microorganism) before detection can be carried out. This means the method is easy and fast.

As the aptamers are specific for the pathogenic or spoilage microorganism being tested—false positives can be kept to a minimum.

Another advantage of aptamers is that they can be stored as a lyophilised powder at room temperature for more than one year. In addition aptamers can recover their native active conformation after denaturation.

Aptamers are more stable at higher temperatures than antibodies, which can give significant advantages.

### Biosensors

Biosensors are devices for the detection of biological analytes. Biosensor applications can differentiate biological recognition elements such as enzymes, antibodies and nucleic acids, to detect the target molecule.

A typical biosensor contains three components: a biological sensing element that can recognise or bind the analyte, a transducing element which converts the detection event into a measurable signal, and a display that transforms the signal into a digital format.

The sensing element primarily defines the selectivity and sensitivity of the biosensor.

The detection of the analytes is usually based on sensing the analytes with either an electrical (Liss et al., (2002), Analytical Chemistry, 74, 4488-4495; Tombelli et al., 2005 Biosensors and Bioelectronics, 20, 2424-2434; Liu et al., 2009 Electrochimica Acta, 54, 6207-6211) or optical (Baldrich et al., 2004 Analytical Chemistry, 76, 7053-7063; Wang et al., 2007b Analytical and Bioanalytical Chemistry, 389, 819-825; Lautner et al., 2010 The Analyst, 135, 918-9; Ohk et al., 2010 Journal of Applied Microbiology, 109, 808-817) readout, each of these references in incorporated herein by reference.

A problem in development of biosensors is the failure of most biomolecules to produce an easily measured signal upon target binding. For example, antibodies normally do not change their shape or dynamics when they bind to their target.

Biosensor technology is currently creating interest because it promises equally reliable results in a shorter time compared to more traditional detection methods such as PCR, colony count, and ELISA.

Some examples of rapid biosensor platforms for the detection of bacteria will be now introduced. A highly sensitive and specific RNA biosensor for the rapid detection of viable E. coli in water was developed by Baeumner et al. (2003) Biosensors and Bioelectronics, 18, 405-413. This biosensor can detect as few as 40 bacterial cells in 15-20 minutes. The detection of this portable, inexpensive and very easy to use biosensor was based on the amplification of mRNA. A biosensor to detect food-borne pathogens was developed by Muhammed-Tahir & Alocilja (2003) Biosensors and Bioelectronics, 18, 813-819. It was a conductometric biosensor that provided a specific, sensitive, low volume, and near real-time detection mechanism for food-borne pathogens. The biosensor is based on electrochemical immunoassay which are biosensors constructed with antibodies as biological elements, attached to an electrochemical transducer. In their study the enterohemorrhagic E. coli O157:H7 and Salmonella ssp. which are of concern to biosecurity were used. It was suggested that the method can be changed for detection of other food-borne pathogens by changing the specificity of the antibodies.

It is envisaged herein that antibodies used as biological elements may be replaced with aptamers in biosensor applications. This change enables a rapid method to detect pathogenic bacteria. The advantages of using aptamers over antibodies include the lower costs of production and there are no ethical issues when aptamers are used because they can be produced by a chemical synthesis where no animals or animal cells are needed.

Aptamer-based biosensors, aptasensors, can be used for the detection of pathogenic micro-organisms and viruses.

In general, aptamers can be a very good substitute for antibodies because they are easy to handle and they are stable compared to biologically generated proteins.

Aptasensors also provide an advantage in chemical stability compared to antibody based affinity biosensors (Liu et al., 2010 Electrochimica Acta, 54, 6207-6211).

Liss et al. (2002) Analytical Chemistry, 74, 4488-4495 demonstrated that the performance of the aptamers as immobilised ligands in biosensor application can be as good as antibodies when considering the sensitivity and specificity. Better performance was also found in terms of stability and reusability of the biosensor as the aptamer biosensor was found to be relatively heat resistant and stable over several weeks and it can tolerate repeated affine layer regeneration after ligand binding.

In one embodiment the device and/or biosensor according to the present invention may comprise elements from biosensors and/or aptasensors as disclosed herein.

In one embodiment the aptamers according to the present invention may be used in any known biosensor and/or aptasensor.

### Aptasensors—Aptamer Based Biosensors

Aptamers could be used as biological recognition elements of biosensors. That an aptamer has bound to its target does not mean it can be used in a biosensor as it is necessary to have a measurable signal from a binding event between the aptamer and the target. When the aptamers bind to their target they usually undergo significant conformational changes. This has been suggested to be one of the key factors when designing the aptamer based biosensors (Wang et al. Analytical and Bioanalytical Chemistry, 389, 819-825 2007b; Zhang et al., 2008 Small, 4, 1196-1200).

Aptasensors have created interest because they are easy to handle and they are stable compared to biologically generated proteins. They are also chemically more stable than antibody-affinity biosensors.

The aptamer based biosensors often use immobilised aptamers as recognition elements for the target molecules. The most popularly used electrode material is gold where the thiolated DNA/RNA strands, in this case aptamers, can be immobilised via strong Au-S linkage (Herne et al., 1997 Journal of American Chemistry Sociaty, 119, 8916-8920; Steel et al., 1998 Analytical Chemistry, 70, 4670-4677). A streptavidin-biotin linkage has also been used (Hamula et al., 2008 Trends in Analytical Chemistry, 25, 681-691; Joshi et al., 2009 Molecular and Cellular Probes, 23, 20-28).

Both, RNA and DNA aptamers have been used in biosensors. It has been established that the unmodified RNA aptamer based biosensors can be used only for a single measurement in biological media because of the degradation of the RNA by the ribonucleases (McCauley et al., 2003 Analytical Biochemsitry, 319, 244-250.). DNA aptamer based assays have been shown to be reusable with minimal or no change in sensitivity (Lee & Walt, 2000 Analytical Biochemistry, 282, 142-146; Liss et al. Analytical Chemistry, 74, 4488-4495, 2002; Minunni et al., 2004 Biosensors and Bioelectronics, 20, 1149-1156; Liu et al., 2009 Electrochimica Acta, 54, 6207-6211). These findings would suggest the DNA based aptamers are more suitable for the biosensor applications even though the stability of RNA aptamers could be improved with modification of the aptamer structure or by adding ribonuclease inhibitors. Thrombin binding aptamers are well established and thrombin is the most commonly used analyte when developing aptamer based biosensors (Hall et al., 2009 Biotechnology and Bioengineering, 103, 1049-1059; Torres-Chavolla & Alocilja, 2009 Biosensors and Bioelectronics, 24, 3175-3182).

Also the first reported aptamer based biosensor was used for thrombin detection (Potyrailo et al., 1998 Analytical Chemistry, 70, 3419-3425). In the study of Potyrailo et al. (1998) anti-thrombin aptamers were fluorescently labelled and immobilized on a glass support. The binding of thrombin to the aptamers was demonstrated by detecting the changes in the evanescent-wave-induced fluorescence anisotropy of the immobilised aptamer.

There are already a wide range of different aptamer based biosensor techniques published in the scientific literature. A skilled person will appreciate that the aptamers according to the present invention may be used any of known biosensor or aptasensor.

By way of example only some of the recent aptamer based biosensors will be introduced.

The aptamers of the present invention may be used with one or more of these biosensors.

The term biosensor as used herein may be any one of the biosensors or aptasensors taught herein and which comprises the aptamers of the present invention.

The aptamer biosensors are roughly divided into two different groups: the biosensors where the interaction between the aptamer and the analyte is detected by optical readout, or by electrochemical readout.

### Optical Platforms

Optical platforms use colour or fluorescence labels in detection of the aptamer binding to the analyte. Biosensors based on surface plasmon resonance (SPR) can be used for label-free analysis of biomolecular interactions, providing data on selectivity, affinity and kinetics (Näslund et al., 2006 Nature Methods Application Notes, 14-16). SPR has been used to study the interactions between the aptamers and their targets (Baldrich et al. 2004 Analytical Chemistry, 76, 7053-7063; Tombelli et al., 2005 Biosensors and Bioelectronics, 20, 2424-2434; Wang et al., 2007b Analytical and Bioanalytical Chemistry, 389, 819-825; Lautner et al., 2010 The Analyst, 135, 918-926). This technique relies on the change of the optical parameter upon changes in the layer closest to the sensitive surface. Other platforms such as fibre optic biosensors (Lee & Walt, 2000 (supra); Ohk et al., 2010 (supra)), colorimetric sensors (Liu & Lu, 2006 Angewandte Chemie International Edition, 45, 90-94), fluorescence based biosensors, where the fluorescence signal is due to the conformation change of the aptamer (Nutiu & Li, 2003 Journal of American Chemical Society, 125, 4771-4778; 2004 A European Journal, 10, 1868-1876; Hall et al. 2009 (supra); Tuleuova et al., 2010 Analytical Chemistry, 82, 1851-1857), or fluorescence polarisation (McCauley et al., 2003 (supra)), and gold nano-particle based biosensors (Wang et al. 2007b (supra); Zhang et al., 2008 (supra)) have been developed.

### Surface Plasmon Resonance

SPR imaging has been used on several occasions to study interactions between the aptamers and their targets. Tombelli et al. (2005) (supra) used previously reported RNA aptamers (Yamamoto et al., 2000, Genes Cells, 5, 371-388) as a biorecognition element to develop aptasensors for the detection of human immunodeficiency virus type 1 (HIV-1) Tat protein. In their platform the aptamers were immobilised on a gold surface and SPR was used to detect the interaction between the aptamer and protein. Analytical performance (sensitivity, reproducibility and specificity) of SPR-based biosensor was studied and the immobilisation of the aptamer resulted in a very reproducible step. High selectivity was also obtained for this biosensor. Wang et al. (2007b) (supra) developed an SPR biosensor for human immunoglobulin E (IgE) detection. Thrombin aptamer in Biacore platform, that uses SPR, was extensively studied and optimised by Baldrich et al. (2004) (supra). The thiolated trombin binding aptamers were immobilised at gold surfaces by self-assembly and the aptamer thrombin interactions were studied by SPR. They found out that different parameters, such as immobilisation strategy, incubation time and temperature, and buffer composition should be optimised for each aptamer. They also suggested that all the aptamers are unique to its structure and a considerable study of assay parameters is necessary for the elucidation of the optimal system.

An SPR based DNA aptamer biosensor for the detection of apple stem pitting virus (ASPV) coat proteins PSA-H and MT32 was proposed by Lautner et al. (2010) (supra). In their aptasensor, the thiolated aptamers were immobilised onto a gold sensor chip surface and different parameters affecting this binding, such as the aptamer flanking, surface coverage, and type of spacer molecules, were identified and their influence was determined. Various concentrations of the target proteins were exposed to the sensor chip and dissociation constants indicating affinity between aptamer and protein were generated with 55 nM for MT32 aptamer and 8 nM for PSA-H aptamer. The aptasensor was shown to be specific to its target proteins and the SPR signal increased when higher amounts of virus were exposed to the sensor chip.

### Fluorescence Platform

Fluorescence signalling aptamers have been used in many aptasensors and different fluorescence based techniques have been extensively reviewed by Nutiu & Li (2005) Methods, 37, 16-25. The same research group developed a structure-switching signalling aptamers (Nutiu & Li, 2003; 2004 (supra)). Their non-fluorescent aptamer can be turned into fluorescencesignalling reporter when the target molecule is available. In their study the aptamer binds to a DNA sequence modified with a quencher (QDNA) in the absence of the aptamer target. When the system is exposed to the target analyte the aptamer binds to the target instead of the QDNA. This conformation change means that the quencher effect of the QDNA disappears and the fluorescence signal of the aptamer can be detected. Similar to structure-switching aptasensors, aptamer beacons (Hall et al., 2009, supra) that work in similar way to the molecular beacons (Tyagi & Kramer, 1996 Nature Biotechnology, 14, 303-308) has been reported. An interaction of the aptamer beacons with the analytes leads to a separation of fluorophore and quencher. Hall et al. (2009) (supra) generated a series of thrombin aptamer beacons. A fluorophore and quencher were included in the aptamer. An addition of thrombin leads to a conformation change of the aptamer and a separation of the fluorophore from the quencher. They developed two different thrombin aptamer beacons that had fast activation rates at 25° C. An aptamer array sensor was developed for the multiplex detection of four analytes in biological matrix such as human serum or cellular extract (McCauley et al., 2003 (supra)).

### Colorimetric Platform

The optical colorimetric signalling has been reported extensively by Liu & Lu (2006) (supra). They developed a fast colorimetric sensor based on the disassembly of nanoparticle aggregates linked by aptamers. In their study, the sensors were developed to detect adenosine and cocaine. The adenosine detecting biosensor was made of nanoparticles containing three components: gold nanoparticles functionalised with 3′-thiolmodified DNA, or 5′-thiol-modified DNA, and a linker DNA molecule. A similar gold nanoparticle-based (AuNPs) simple readout technique was developed to detect target analytes with aptamers by naked eye (Wang et al. 2007b (supra)). The technique was simple in design as no oligonucleotide labelling or AuNPs modification was needed AuNPs have also been used in detection of small molecules, such as adenosine and potassium, by Zhang et al. (2008) (supra). Their strategy relies on the size-dependent SPR properties of AuNPs probes and aptamers.

### Fibre-Optic Platform

A fiber-optic biosensor to detect thrombin was developed by Lee & Walt (2000) (supra). The aptamers were immobilized on the surface of silica microspheres and the binding of the protein was monitored in a microarray system. A similar fibre optic based aptasensor was developed by Ohk et al. (2010) (supra). Their antibody-aptamer functionalized fibre-optic biosensor can be used in the detection of Listeria monocytogenes from food. The sandwich fiber-optic biosensor was based on an aptamer, specific for an invasion protein of L. monocytogenes together with an antibody. The antibody was immobilised on a surface to capture the target bacteria and aptamer was used as fluorescence-labelled reporter.

### Electrochemical Platforms

Aptamer based electrochemical biosensors have been extensively reviewed byseveral authors (e.g Willner & Zayats, 2007 Angewandte Chemie International Edition, 46, 6408-6418; Lee et al., 2008 Analytical and Bioanalytical Chemistry, 390, 1023-1032; Cheng et al., 2009 Bioelectrochemistry, 77, 1-12; Sassolas et al., 2009 Electroanalysis, 21, 1237-1250). Typical, electrochemical aptasensor use an electrode surface as the platform to immobilise the aptamers. The binding of the analyte is then monitored based on electrical current variations.

Detection methods, such as for example; quartz crystal microbalance (QRM) (Liss et al., 2002 Analytical Chemistry, 74, 4488-4495; Tombelli et al., 2005 (supra); Hianik at al., 2007 Bioelectrochemistry, 70, 127-133), electrochemical impedance spectroscopy (EIS) (Xu et al., 2005 Analytical Chemistry, 77, 5107-5113; Min et al., 2008 Biosensors and Bioelectronics, 23, 1819-1824; Liu et al., 2009 Electrochimica Acta, 54, 6207-6211; Ho, et al., 2012 Analytical Chemistry, 84, 4245-4247) and several different voltammetry methods, such as cyclic voltammetry (CV) (Cheng et al., 2007 Biochemical and Biophysical Research Communications, 357, 743-748; Liu et al., 2009 (supra)) and square wave voltammetry (SWV) (Liu et al., 2009 (supra)) have been used for detecting the binding events between aptamers and analytes. The sensors are normally based on the changes in aptamer configuration, conformation, and conductivity of the aptamer-containing DNA construct upon binding an analyte, respectively. Someaptasensors based on electrochemical detection are introduced here to give a short overview of the aptamer based electrochemical biosensors.

Although the development of aptamer based biosensors is proceeding (Freeman et al., 2012 Analytical Chemistry, 84, 6192-9198; Kim et al. 2012 Analytical Chemistry, 84, 6192-9198), relatively few aptasensors have been developed for the direct detection of bacterial cells, particularly pathogenic bacterial cells. Offering detection methods with little or no preanalysis preparation, coupled with the potential to detect highly pathogenic organisms, aptamers are emerging as a cost effective tool for use in rapid diagnostics for food quality and assurance.

In one aspect of the present invention there is provided a aptasensor comprising the aptamers according to the present invention.

### Isolated

In one aspect, preferably the nucleic acid sequence according to the present invention is in an isolated form. The term “isolated” means that the sequence is at least substantially free from at least one other component with which the sequence is naturally associated in nature and as found in nature. The sequence of the present invention may be provided in a form that is substantially free of one or more contaminants with which the substance might otherwise be associated. Thus, for example it may be substantially free of one or more potentially contaminating polypeptides and/or nucleic acid molecules.

### Purified

In one aspect, preferably the aptamer according to the present invention is in a purified form. The term “purified” means that the given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### Nucleotide Sequence

The term “nucleotide sequence” as used herein refers to an oligonucleotide sequence or polynucleotide sequence, and variant, homologues, fragments and derivatives thereof (such as portions thereof). The nucleotide sequence may be of synthetic or recombinant origin, which may be single-stranded whether representing the sense or anti-sense strand.

The term “nucleotide sequence” in relation to the present invention includes synthetic DNA and RNA.

Preferably the nucleotide sequence of the aptamer according to the present invention could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

The term “fragment” as used herein may mean a portion of the aptamer sequence taught herein which has the same or better affinity, specificity or functional activity for the target of interest compared with the full sequence. The fragment may be comprised of about 20 nucleotides (e.g. 18-25, preferably 19-21 nucleotides). The fragment preferably has a secondary structure similar to that of the original full length aptamer over the region represented by the fragment.

### Sequence Identity

The present invention also encompasses the use of sequences having a degree of sequence identity or sequence similarity with the nucleic acid sequence(s) of the present invention.

In the present context, a similar sequence is taken to include a nucleotide sequence which may be at least 80%, suitably at least 90% identical, preferably at least 95 or 98% identical to the subject sequence. Typically, the similar sequences will comprise the same or similar secondary structure as the subject nucleic acid aptamer.

In one embodiment, a similar sequence is taken to include a nucleotide sequence which has one or several additions, deletions and/or substitutions compared with the subject sequence.

Sequence identity comparisons can be conducted by eye, or more usually, with the aid of readily available sequence comparison programs. These commercially available computer programs can calculate % identity between two or more sequences.

% identity may be calculated over contiguous sequences, i.e. one sequence is aligned with the other sequence and each base in one sequence is directly compared with the corresponding base in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following residues to be put out of alignment, thus potentially resulting in a large reduction in % identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical bases, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final % identity can be measured in terms of pure identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage identities may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 0 |
| GAP EXTENSION | 0 |
| FOR CLUSTAL | DNA |
| WORD SIZE | 2 |
| GAP PENALTY | 15 |
| GAP EXTENSION | 6.66 |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, suitably over at least 50 contiguous nucleotides.

Suitably, the degree of identity with regard to a nucleotide sequence may be determined over the whole sequence.

Preferably similar or homologous sequences have a similar secondary structure to the original aptamer.

As well as primary sequence it is the secondary structure or conformation of the aptamer which is most likely important for its binding specificity.

Aptamer sequences may be refined (e.g. by random or directed mutagenesis) to alter the base sequence in order to generate aptamer molecules with greater target affinity or specificity.

### Synthetic Nucleic Acids

The nucleic acid aptamers according to the present invention and for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the specificity of the aptamers.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries. In addition, similar sequences should be capable of selectively hybridising to the sequences shown in the sequence listing herein.

Alternatively, such polynucleotides may be obtained by site directed mutagenesis of characterised sequences.

The term “synthetic” as used herein may mean chemically synthesised.

### Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

Preferably, the sequences that are capable of hybridising to the nucleic acid sequences present herein under stringent conditions (e.g. 50° C. and 0.2×SSC {1×SSC=0.15 M NaCl, 0.015 M Na₃citrate pH 7.0}).

More preferably, the sequences that are complementary to the nucleotide sequences presented herein are capable of hybridising under high stringent conditions (e.g. 65° C. and 0.1×SSC {1×SSC=0.15 M NaCl, 0.015 M Na₃citrate pH 7.0}).

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

### Food

The composition of the present invention may be used to detect a microorganism in a food. Here, the term “food” is used in a broad sense—and covers food for humans as well as food for animals (i.e. a feed). In a preferred aspect, the food is for human consumption.

The food may be in the form of a solution or as a solid—depending on the use and/or the mode of application and/or the mode of administration.

The food in which the detection methods can be used may be one or more of: jams, marmalades, jellies, dairy products (such as milk or cheese), meat products, poultry products, fish products and bakery products.

The beverage in which the detection methods may be used include soft drinks, a fruit juice or a beverage comprising whey protein, health teas, cocoa drinks, milk drinks and lactic acid bacteria drinks, yoghurt and drinking yoghurt, calcium fortified soy/plain and chocolate milk, calcium fortified coffee beverage, wine and beer.

### Meat Based Food Product

A meat based food product according to the present invention is any product based on meat. The meat based food product is suitable for human and/or animal consumption as a food and/or a feed. In one embodiment of the invention the meat based food product is a feed product for feeding animals, such as for example a pet food product. In another embodiment of the invention the meat based food product is a food product for humans.

A meat based food product may comprise non-meat ingredients such as for example water, salt, flour, milk protein, vegetable protein, starch, hydrolysed protein, phosphate, acid, spices, colouring agents and/or texturising agents.

A meat based food product in accordance with the present invention preferably comprises between 5-90% (weight/weight) meat. In some embodiments the meat based food product may comprise at least 30% (weight/weight) meat, such as at least 50%, at least 60% or at least 70% meat.

In some embodiments the meat based food product is a cooked meat, such as ham, loin, picnic shoulder, bacon and/or pork belly for example.

The meat based food product may be one or more of the following:

Dry or semi-dry cured meats—such as fermented products, dry-cured and fermented with starter cultures, for example dry sausages, salami, pepperoni and dry ham; Emulsified meat products (e.g. for cold or hot consumption), such as mortadella, frankfurter, luncheon meat and pâté;

Fish and seafood, such as shrimps, salmon, reformulated fish products, frozen cold-packed fish;

Fresh meat muscle, such as whole injected meat muscle, for example loin, shoulder ham, marinated meat;

Ground and/or restructured fresh meat—or reformulated meat, such as upgraded cut-away meat by cold setting gel or binding, for example raw, uncooked loin chops, steaks, roasts, fresh sausages, beef burgers, meat balls, pelmeni;

Poultry products—such as chicken or turkey breasts or reformulated poultry, e.g. chicken nuggets and/or chicken sausages;

Retorted products—autoclaved meat products, for example picnic ham, luncheon meat, emulsified products.

In one embodiment of the present invention the meat based food product is a processed meat product, such as for example a sausage, bologna, meat loaf, comminuted meat product, ground meat, bacon, polony, salami or pate.

A processed meat product may be for example an emulsified meat product, manufactured from a meat based emulsion, such as for example mortadella, bologna, pepperoni, liver sausage, chicken sausage, wiener, frankfurter, luncheon meat, meat pate.

The meat based emulsion may be cooked, sterilised or baked, e.g. in a baking form or after being filled into a casing of for example plastic, collagen, cellulose or a natural casing. A processed meat product may also be a restructured meat product, such as for example restructured ham. A meat product of the invention may undergo processing steps such as for example salting, e.g. dry salting; curing, e.g. brine curing; drying; smoking; fermentation; cooking; canning; retorting; slicing and/or shredding.

In one embodiment the meat may be minced meat.

In another embodiment the food product may be an emulsified meat product.

### Meat

The term “meat” as used herein means any kind of tissue derived from any kind of animal.

The term meat as used herein may be tissue comprising muscle fibres derived from an animal. The meat may be an animal muscle, for example a whole animal muscle or pieces cut from an animal muscle.

In another embodiment the meat may comprise inner organs of an animal, such as heart, liver, kidney, spleen, thymus and brain for example.

The term meat encompasses meat which is ground, minced or cut into smaller pieces by any other appropriate method known in the art.

The meat may be derived from any kind of animal, such as from cow, pig, lamb, sheep, goat, chicken, turkey, ostrich, pheasant, deer, elk, reindeer, buffalo, bison, antelope, camel, kangaroo; any kind of fish e.g. sprat, cod, haddock, tuna, sea eel, salmon, herring, sardine, mackerel, horse mackerel, saury, round herring, Pollack, flatfish, anchovy, pilchard, blue whiting, pacific whiting, trout, catfish, bass, capelin, marlin, red snapper, Norway pout and/or hake; any kind of shellfish, e.g. clam, mussel, scallop, cockle, periwinkle, snail, oyster, shrimp, lobster, langoustine, crab, crayfish, cuttlefish, squid, and/or octopus.

In one embodiment the meat is beef, pork, chicken, lamb and/or turkey.

### Vegetable Based Food Product

The vegetable based product as taught herein may be any vegetable.

Suitably the vegetable based food product as taught herein may be a fermented vegetable product, a brined vegetable, or a pickled vegetable product.

In one embodiment the vegetable based food product as taught herein may be a beverage, for example a beverage containing soya such as a soya vegetable drink.

Suitably the vegetable based food product as taught herein may be a fermented vegetable product such as a sauerkraut fermentation, pickles from fresh, green cucumbers, fermented mixed vegetables or any fermented plant or legumes that can be for example onion, celery, beet, lettuce, spinach, broccoli, cauliflower, mushroom, potatoes, radish, cabbage, peas. It can also be silage.

Suitably the vegetable based food product as taught herein may be bean based, such as cheonggukjang, doenjang, miso, natto, soy sauce, stinky tofu, tempeh for example.

Suitably the vegetable based food product as taught herein may be grain based. In some embodiments the vegetable based food product may be a batter made from rice and lentil (Vigna mungo) prepared and fermented for baking Idlis and Dosas, amazake, beer, bread, choujiu, gamju, injera, makgeolli, murri, ogi, sake, sikhye, sourdough, rice wine, Malt whisky, grain whisky, Vodka, batter.

### Pharmaceutical

Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

The invention will now be described, by way of example only, with reference to the following Figures and Examples.

### EXAMPLES

### Example 1

### Development of Novel Biomolecules Based on DNA (Aptamers)

Novel biomolecules based on DNA (aptamers) have potential applications in the area of food safety and quality assurance. The method for the selection of the aptamers was developed using a previously described selective evolution technique (SELEX). The SELEX procedure by which aptamers are generated offers the prospect of generating biomolecules with specific binding properties; similar to those exhibited by antibodies. These novel molecules can be used as the basis of either simple, rapid assays or real-time monitors of food quality. The application of tools based on aptamers will further help to ensure the supply of safe food and prevent incidents of food poisoning. This technology also offers the prospect of the animal-free alternative to commercial diagnostic procedures that are based on the use of antibodies.

In this study, the selection technique was established and the detection technique developed by selecting the aptamers against non-pathogenic Escherichia coli K12. The same techinque was then used to select the aptamers against the common pathogenic food poisoning bacteria E. coli O157, Listeria monocytogenes and Salmonella typhimurium. The aptamers for both non-pathogenic and pathogenic E. coli and for S. typhimurium were cloned and sequenced.

The results of this study show that the non-pathogenic E. coli aptamers bind specifically to live E. coli K12 bacterial cells and these aptamers were also shown to be suitable for detecting bacterial cells extracted from natural probiotic yoghurt. Preliminary studies have also shown that the identified aptamers for pathogens can also be used to detect live bacterial cells.

A rapid detection method based on the aptamers selected in this study can be developed or the aptamers can possibly be used as a part of an existing detection system.

### 1. Materials and Methods

1.1 Selection of the Aptamers Against Non-Pathogenic E. coli K12

The selection of the aptamers started with a creation of a random DNA library. Non-pathogenic E. coli K12 was first used to establish the method for the aptamer selection against live bacterial cells. A method based on centrifugation was used for the aptamer selection to separate the non-binding molecules from those having the affinity to the structures on bacterial cell surface. The aptamers were then cloned and sequenced and the binding of these aptamer sequences were tested by using a method based on fluorescence. The binding was also visualised by a microscope.

A natural yoghurt containing Lactobacillus acidophilus and Bifidobacterium spp was used as an example of a food matrix to test the aptamers. The yoghurt was spiked with E. coli K12 cells and the bacterial cells were roughly separated from yoghurt. The aptamers were then used to detect the cells.

### 1.2 Selection of the Aptamers Against Pathogenic Bacteria

Using the selection process previously developed the aptamers were selected against pathogenic Listeria monocytogenes and Salmonella typhimurium. The aptamers were also selected against E. coli O157 but the selection was done from the pool of E. coli K12 binding aptamers instead of DNA library. E. coli O157 and S. typhimurium aptamers were cloned and the sequences analysed.

### 2. Results

2.1 Selection of the Aptamers Against Non-Pathogenic E. coli K12

Aptamer selection and detection methods were established by selecting the aptamers against non-pathogenic E. coli K12. Aptamers were cloned and four clones were selected for the affinity tests. The sequences of these aptamers can be seen in Table 2.1. As an example, a predicted aptamer structure for aptamer 6AptK12 is presented in the table.

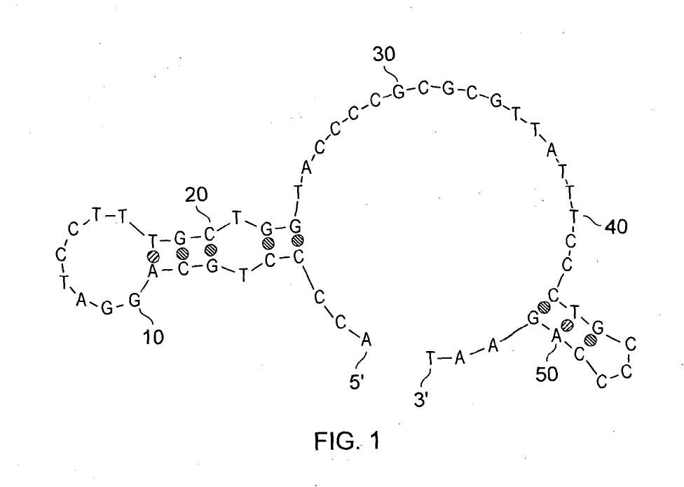

TABLE 2.1
Sequences for the synthesised FAM-labelled
aptamers, the predicted secondary structure
for aptamer 6AptK12 is shown in FIG. 1.

| Aptamer | Nt | Sequence |
|---|---|---|
| 1AptK12 | 54 | 5′FAM-ACCCCTGCAGGATCCTTTGCTGGTACCCCGCGC |
| | | GTTATTTCCCTGCCCCAGAAT-3′ (SEQ ID No. 1) |
| 2AptK12 | 51 | 5′FAM-CCCTCCCTCATCCGTTGTCTCGCTCAGAGTATC |
| | | GCTAATCAGTCTAGAGGG-3′ (SEQ ID No. 2) |
| 4AptK12 | 54 | 5′FAM-ATTCTGGGGCCCTCTAGACTGATTAGCGATACT |
| | | ACTTAACCTGCATGCAGGGGT-3′ (SEQ ID No. 3) |
| 6AptK12 | 64 | 5′FAM-ACCCCTGCAGGATCCTTTGCTGGTACCGCGTTA |
| | | TGGGAAAATCAGGAGAGAGGGGCCCCAGAAT-3′ |
| | | (SEQ ID No. 4) |

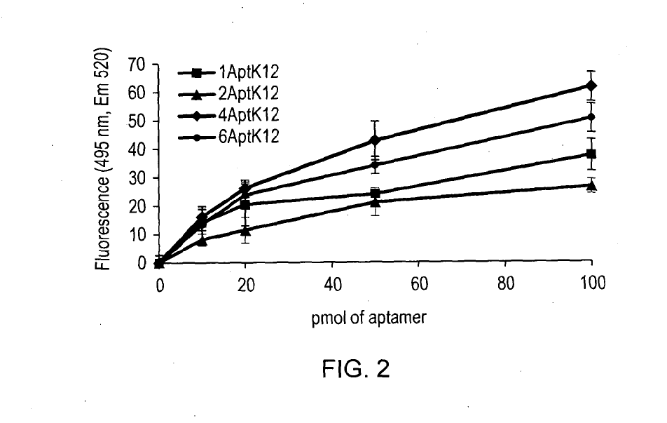

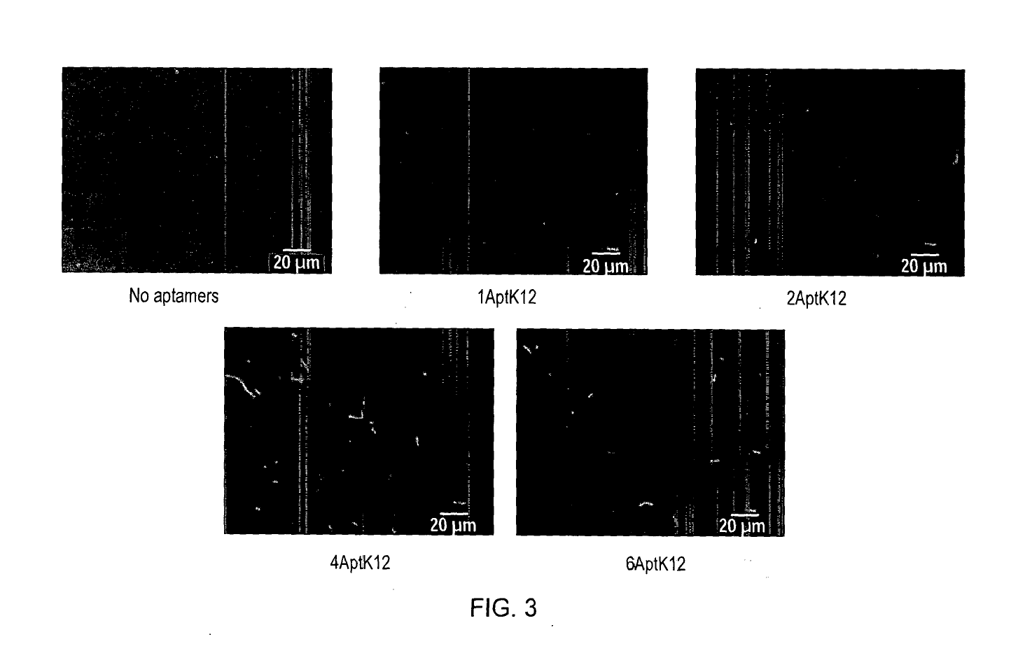

The binding affinity and specificity of the cloned aptamers were tested using fluorescent-labelled aptamers combined with fluorimetry analysis (FIG. 2) and microscopy (FIG. 3).

The results in FIG. 2 shows that the greater the number of bound aptamers the higher the fluorescence. This means that the aptamers have bound on the surface of E. coli K12. It can be seen that the two strongest binding aptamers are 4AptK12 and 6AptK12.

The samples were also visualised under the microscope (FIG. 3) in the images it can be seen, that when the aptamers are added, some of the bacterial cells can be seen as bright green spots.

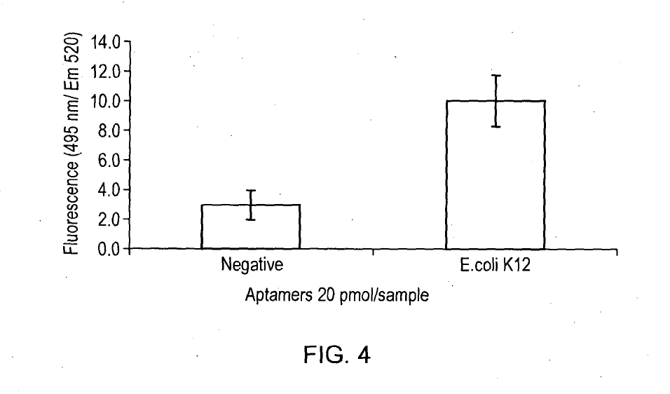

A natural yoghurt containing Lactobacillus acidophilus and Bifidobacterium spp was used as an example food matrix to test the specificity of the aptamers. The yoghurt was spiked with E. coli K12 and a mixture of specific aptamers was used to detect the bacterial cells by fluorimetry analysis. The fluorescence values are presented in FIG. 4. It can be seen in the figure that the fluorescence is significantly higher in the E. coli K12 sample than in the negative sample where E. coli K12 has not been added to the yoghurt.

### 2.2 Selection of the Aptamers Against Pathogenic Bacteria

Aptamers against E. coli O157 and S. typhimurium were selected and cloned. A list of cloned sequences can be seen in Table 2.2.

TABLE 2.2
Sequences for the FAM-labelled E. coli O157
(497)and S. typhimurium (223) aptamers.

| Aptamer | Nt | Sequence |
|---|---|---|
| 1Apt497 | 42 | 5′FAM-CCTGCATGCCCAGTAAGCGGTACCAGCAAAGG |
| | | ATCCTGCAGG-3′ (SEQ ID No. 5) |
| 2Apt497 | 60 | 5′FAM-ATTCTCCTTAGCCATAAATTACGGAGCGGATG |
| | | AGGTACCAGCAAAGGATCCTGCAGGGGT-3′ |
| | | (SEQ ID No. 6) |
| 4Apt497 | 50 | 5′FAM-CCCTCTAGACTGATTAGCGATACTCTCCCACCT |
| | | ACGCCTTAACTTTTCCA-3′ (SEQ ID No. 7) |
| 2Apt223 | 45 | 5′FAM-ATCCTTTGCTGGTACCTAGAAGCCGGCCGTAGA |
| | | GGAGGAAAGGAT-3′ (SEQ ID No. 8) |
| 3Apt223 | 50 | 5′FAM-CCCTGCAGGATCCTTTGCTGGTACCAGGGAAAT |
| | | CGTAGTTGATTACGATT-3′ (SEQ ID No. 9) |
| 5Apt223 | 35 | 5′FAM-GGGGCTGAGTATCGCTAATCAGTCTAGAGGGCC |
| | | CC-3′ (SEQ ID No. 10) |

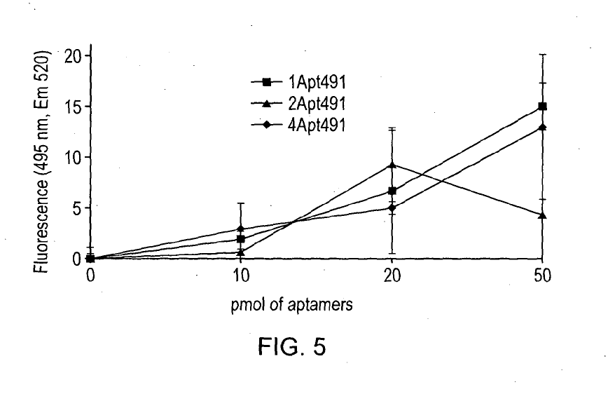

Binding of these six fluorescent-labelled aptamers (Table 2.2) was tested. The results from fluorimetry analysis for E. coli O157 aptamers can be seen in FIG. 5. It can be seen that the fluorescence is higher when more aptamers have been added. Despite the high error bar for these samples the overall results show that some aptamer binding for E. coli O157 can be detected.

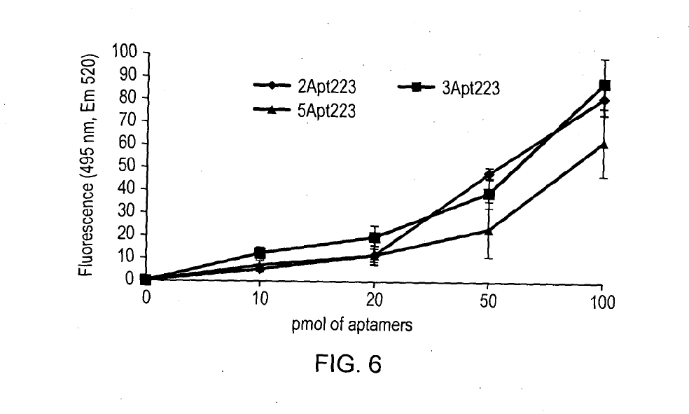

In FIG. 6 are the results for fluorimetry analysis for S. typhimurium binding aptamers. It can be seen that the greater the number of bound aptamers the higher the fluorescence. These results demonstrate that the Apt223 aptamers are binding to S. typhimurium.

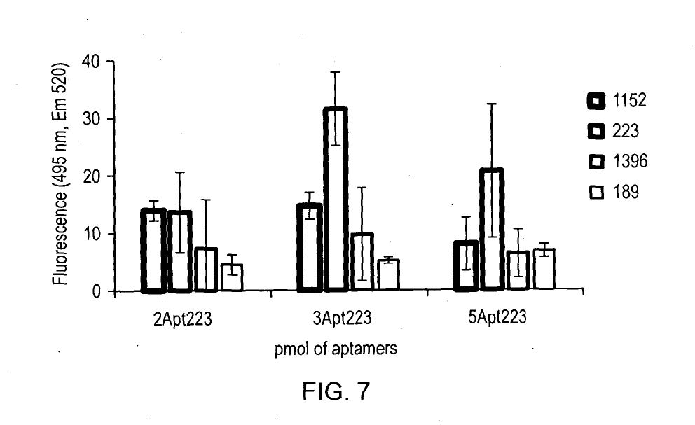

The specificity of S. typhimurium binding aptamers (2Apt223, 3Apt223 and 5Apt223) was tested. The aptamers were incubated with gram negative S. enteritidis and E. coli K12 and gram positive Lactobacillus plantarum. S. typhimurium was used as a positive control. The fluorescence values are shown in FIG. 7. It can be seen that the aptamer 3Apt223 has the strongest binding and is the most specific aptamer from these three S. typhimurium aptamers.

### 3. Conclusions

The selection method for aptamers against live bacterial cells was developed. Non-pathogenic E. coli K12 was used as a model organism for the method development. A fluorimetry method was used to detect the binding of the aptamers to their target organism. In this study, aptamers were selected to non-pathogenic E. coli K12 and pathogenic E. coli O157, L. monocytogenes and S. typhimurium. Aptamers against E. coli K12, E. coli O157 and S. typhimurium were cloned and sequenced and the binding of these aptamers was demonstrated.

### Example 2

### General Methods for Aptamer Identification

### Materials and Methods

### 2.1 Equipment

Bio-Rad Power supply Model 1000/500—Rich-Mond Agencies Ltd., UK;

Bio-Tek Synergy HT Multi-detection Microplate reader—Labtech International Ltd., UK Eppendorf DNA LoBind tubes—Fischer Scientific, UK

Gen5™ 1.07 Data collection and Analysis Software—BioTek Instruments Inc., UK

Hermle Z 323 K Refrigerated centrifuge, fixed angle rotor (24×1.5 mL)—VWR International Ltd., UK

Mastercycler gradient—Eppendorf, USA

MultiScreen™_{HTS} Vacuum Manifold—Millipore Ireland BV

MultiScreen Filter plates with Durapore® Membrane, 0.45 μm Hydrophil Low protein binding—Millipore Ireland BV

Nikon Eclipse TE2000-U Microscope System—Nikon Corporation Ltd., UK—IPLab™ 4.0 Software—Scanalytics, Inc., USA

Olympus BX41 Clinical Microscope—Olympus Corp.—Olympus U-RFL-T Burner—Olympus ColorView Soft Imaging System—CellF 2.7 Cell Imaging Software for Life Science Microscopy—Olympus soft imaging solutions GmbH

Quantity One® 4.6.3. 1-D Analysis Software—BioRad Laboratories Inc., UK

Syngene Bioimaging GeneFlash—Synoptics Ltd., UK

Techne TC-3000 Thermal cycler—Bibby Scientific Ltd., UK

UV transilluminator—BioRad Laboratories Ltd., UK

### 2.2. Materials

### 2.2.1. Reagents

10% Novex® TBE Gel—Invitrogen, Life technologies Ltd., UK

ABTS (2,2′-azino-bis(3-ethylbenzthiazoline-6-sulphonic acid))—Sigma-Aldrich Co., UK

Agar No. 1 Bacteriological MC2—LabM Ltd., UK

Agarose—Fisher Scientific, Thermo Fisher Scientific Ltd., UK

Ampicillin—Sigma-Aldrich Co., UK

Bacto™ Agar—BD Biosciences, BD Co., USA

Bovine Serum Albumin (BSA)—Invitrogen, Life technologies Ltd., UK

D-Biotin (Vitamin B7)—Fisher Scientific Ltd., UK

DNA Loading Dye (6×)—Fermentas, Thermo Fisher Scientific Inc., UK

DTT (Dithiothreitol)—Promega Co., UK

EcoRI—Invitrogen, Life Technologies Ltd., UK

EDTA (Ethylenediaminetetraacetic acid)—Sigma-Aldrich Co., UK

Ethidium bromide (EtBr; 3,8-Diamino-5-ethyl-6-phenylphenanthridinium bromide)—Fluka, Sigma-Aldrich Co., UK

FAM™ (6-Carboxyfluorescein)—Applied Biosystems, Life Technologies Ltd., UK

FluoSpheres NeutrAvidin labelled microspheres, 0.2 μm Yellow-Green—Invitrogen, Life Technologies Ltd., UK

GelRed—Biotium Inc., USA

illustra PuReTaq Ready-To-Go™ PCR Beads—GE Healthcare, Life Sciences, UK

IPTG (Isopropyl β-D-1-thiogalactopyranoside), Dioxane-Free—Promega Co., UK

LA agar (Elliker Broth)—Fluka, Sigma-Aldrich Ltd.

LIVE/DEAD® BacLight™ Bacterial viability kit—Invitrogen, Life technologies Ltd., UK

MES (4-morpholine ethanesulfonic acid)—Sigma, Sigma-Aldrich Co., UK

Microplate Black—Sterilin Ltd., Thermo Fisher Scientific Inc., UK

Mini Sizer 50 bp DNA Ladder—Norgen Biotek Corp. CA

Nutrient agar—LabM Ltd., UK

Nutrient Broth—Oxoid, Thermo Fisher Scientific Inc., UK

PCR 100 bp Low Ladder—Sigma, Sigma-Aldrich Co., UK

PCR Sizer 100 bp DNA Ladder—Norgen Biotek Corp., CA

PCR Supermix High Fidelity (HIFI)—Invitrogen, Life technologies Ltd., UK

pGEM®-T Easy Vector System—Promega Co., UK

PureLink™ Quick Plasmid Miniprep Kit—Invitrogen, Life technologies Ltd., UK

QIAquick gel extraction kit—Qiagen, UK

QIAquick® Spin PCR product purification kit—Qiagen, UK

Spin column PCR purification kit—NBS Biologicals Ltd., UK

Streptavidin peroxidase from Streptomyces avidinii—Sigma, Sigma-Aldrich Co., UK

Tris-Borate-EDTA buffer (TBE)—Invitrogen, Life technologies Ltd., UK

triSodium Citrate—Sucrechem products Ltd., UK

Trizma Base—Sigma, Sigma-Aldrich Co., UK

Tryptic Soy Broth (CASO)—Merk, Merc & Co., Inc., USA

Tryptone—Oxoid, Thermo Fisher Scientific Inc., UK

X-Gal—Promega Co., UK

Yeast extract—Oxoid, Thermo Fisher Scientific Inc., UK

Yeast tRNA—Invitrogen, Life technologies Ltd., UK

Yeo Valley Organic Natural Probiotic Yoghurt—YeoValley organic, UK

### 2.2.2. Bacterial Strains

Bacillus subtilis—NCTC 10400—publically available from the Health Protection Agency.
Escherichia coli K12—ID LZB035—publically available from Blades Biological Ltd, Cowden, Edenbridge, Kent, TN8 7DX, UK
Escherichia coli O157 DCS 497—Danisco A/S, Denmark
Salmonella typhimurium—NCTC12116 (now called Salmonella enterica subsp. enterica)—publically available from the Health Protection Agency.
Salmonella typhimurium DCS 223—Danisco A/S, Denmark
Salmonella enteritidis DCS 1152—Danisco A/S, Denmark
Staphylococcus aureus—publically available from the Health Protection Agency
Lactobacillus bulgaricus—ID LZB045—publically available from Blades Biological Ltd, Cowden, Edenbridge, Kent, TN8 7DX, UK
Lactobacillus plantarum DCS 189—Danisco A/S, Denmark
Listeria innocua DCS 17—DSMZ 20649—publically available from Leibniz Institut DSMZ—Deutsche Sammlung von Mikroorganismen und Zellkulturen GmbH, Inhoffenstraβe 7B, 38124 Braunschweig, Germany or at www.dsmz.de (see http://www.dsmz.de/catalogues/details/culture/DSM-20649.html?tx_dsmzresources_pi5%5BreturnPid%5D=329)
Listeria monocytogenes DCS 489—NCTC 12426—publically available from the Health Protection Agency (see http://www.hpacultures.org.uk/products/bacteria/detail.isp?refId=NCTC+12426&collection=nctc)
Listeria monocytogenes DCS 490—Danisco A/S, Denmark

JM109 Competent cells, High Efficiency—Promega Co., UK

One Shot® MAX Efficiency® DH5α-T1® Chemically competent—Invitrogen, Life technologies Ltd., UK

Specific strains of Salmonella enterica serotype typhimurium are publically available and can be purchased through e.g. ATCC-LGC Standards (http://www.lgcstandards-atcc.org), such as, e.g., the strains having ATCC Number 6994, 7832, 13311, 14028, 15277, 19585, 23555, 23564, 23565, 23566, 23567, 23591, 23592, 23593, 23594, 23595, 23952, 23853, 23854 or 23855. Other sources for obtaining specific strains are e.g. Leibniz-Institut DSMZ—Deutsche Sammlung von Mikroorganismen und Zellkulturen GmbH (http://www.dsmz.de) or Agricultural Research Service Culture Collection (http://nrrl.ncaur.usda.gov/).

Specific strains of Escherichia coli O157 are publically available and can be obtained through for example ATCC-LGC Standards (http://www.lgcstandards-atcc.org), such as, e.g. the strains having ATCC Number 35150, 43888, 43889, 43894 and 43895. Other sources for obtaining specific strains are e.g. Leibniz-Institut DSMZ—Deutsche Sammlung von Mikroorganismen und Zellkulturen GmbH(http://www.dsmz.de) or Agricultural Research Service Culture Collection (http://nrrl.ncaur.usda.gov/).

Details of the Heath Protection Agency can be found at www.hpacultures.org.uk.

### 2.2.3. Oligonucleotides

Oligonucleotides, sequencing and labelled aptamers were obtained from Eurofins MWG Operon. The oligonucleotides and the aptamers were analysed with Oligoanalyzer 3.1 and UNAFold (IDT—Integrated DNA technologies Inc., USA).

### DNA Library:

5′-ACC CCT GCA GGA TCC TTT GCT GGT ACC 40×N AGT
ATC GCT AAT CAG TCT AGA GGG CCC CAG AAT-3′

Primers

Forward PR1:
5′-ACC CCT GCA GGA TCC TTT GCT GGT ACC-3′
Reverse PR2:
5′-ATT CTG GGG CCC TCT AGA CTG ATT AGC
GAT ACT-3′

Biotin labelled primers

PR1BIO:
5′BIO-ACC CCT GCA GGA TCC TTT GCT GGT ACC-3′
PR2BIO:
5′BIO-ATT CTG GGG CCC TCT AGA CTG ATT AGC
GAT ACT-3′

Fluorescence FAM-labelled primers

PR1FAM:
5′FAM-ACC CCT GCA GGA TCC TTT GCT GGT ACC-3′
PR2FAM:
5′FAM-ATT CTG GGG CCC TCT AGA CTG ATT AGC
GAT ACT-3′

Sequencing primers:

SP6r:
5′-TAT TTA GGT GAC ACT ATA G-3′
T7:
5′-TAA TAC GAC TCA CTA TAG GG-3′

### 2.2.4. Buffers and Solutions

All buffers were sterilised by autoclaving at 121° C. for 15 min.

1× Binding buffer for aptamer selection by centrifugation (BB)
50 mM Tris-Cl, pH 7.4
5 mM KCl
100 mM NaCl
1 mM MgCl₂
   1× Binding buffer for aptamer selection by filtration (BBf)
20 mM Tris-Cl, pH 7.5
45 mM NaCl
3 mM MgCl
1 mM EDTA
1 mM DTT

The stock solutions for the binding buffers were first prepared and autoclaved before making up to a final volume.

0.05 M Citrate buffer, pH 4.0
9.61 g Citrate acid (anhydrous)
1000 mL dH₂O
   Elution buffer (filter selection)
7 M Urea
100 mM MES
3 mM EDTA
   1×PBS (Tris Phosphate buffered saline), pH 7.2
8 g NaCl
0.2 g KCl
1.44 g Na₂ HPO₄
0.24 g KH₂PO₄
1000 mL H₂O
   20× Saline Sodium Citrate buffer (SSC), pH 7.9
3 M NaCl
300 mM triSodium citrate

### 1× TE-Buffer (Tris-EDTA), pH 8.0

10 mM Tris
1 mM EDTA
   50× Tris base-acetic acid-EDTA buffer (TAE)
242 g Trizma base
57.1 mL Glacial acetic acid
100 mL 0.5 M EDTA

### 2.2.5. Bacterial Growth Media

Plate Count Agar (PC-Agar)
Tryptone solution, pH 7.0
   2.5 g Tryptone
   1.25 g Yeast Extract
   0.5 g Glucose
   500 mL dH₂O
Agar solution:
   6 g Agar
   500 mL dH₂O

The tryptone solution pH was adjusted and the solution was mixed with the agar. The mixture was autoclaved and plated.
Luria Broth (LB)
   5 g Yeast extract
   10 g Tryptone
   5 g NaCl
   1000 mL dH₂O

For selective antibiotic containing media, filter sterilised antibiotic was added into the autoclaved solution (25 μg/mL, 50 μg/mL or 100 μg/mL).

Indicator plates (Selective LB-Agar+ampicillin, IPTG and X-Gal)
5 g Yeast extract
10 g Tryptone
5 g NaCl
1000 mL dH₂O
pH of the solution was adjusted to 7.0 and 14 g of agar was added. Sterilised ampicillin 100 μg/mL, IPTG 0.5 mM and X-Gal 80 μg/mL were added to autoclaved solution.

### S.O.C Medium

2 g Tryptone
0.5 g Yeast Extract
1 mL NaCl
0.25 mL KCl
100 mL dH₂O
Sterile filtered Mg²⁺ solution and sterile filtered glucose solution in a final concentration of 20 mM. Each was added to autoclaved solution (pH 7.0).

### 2.3. Methods

### 2.3.1. Polymerase Chain Reaction

PCR reaction was performed by using Ready-to-Go PCR beads with 25 pmol of reverse PR1 and forward PR2 primers and 1 μl of template DNA. The amplification parameters were 5 min at 94° C. for initial denaturation, denaturation at 94° C. for 45 s, annealing at 62° C. for 45 s, and elongation at 72° C. for 45 s unless otherwise stated. The final elongation was 7 min at 72° C. Denaturation, annealing, and elongation were initially repeated for 15 or 20 cycles depending on the reaction. The PCR products were separated on agarose gel and the products were purified from the gel with a gel extraction kit or a spin column PCR purification kit by following the manufacturer's protocols.

### 2.3.2. Electrophoresis

The sizes of the PCR products were estimated on 2% agarose gel in 1×TAE buffer and 0.01% GelRed or 0.05% EtBr, or on polyacrylamide gel in 1×TBE buffer and post staining the gel with GelRed. PCR samples (3 μl) were mixed with 6× loading dye and topped up with the water to achieve 1× loading dye solution before the samples were applied in the wells. The agarose gels were run for 40-60 minutes with an electric field of 80V to 210V depending on the size of the gel. Polyacrylamide gels were run for 1 h in an electric field of 170V. The gels were visualised by UV transillumination and the pictures were taken and analysed.

### 2.3.3. DNA Library Production

The aptamers were selected from a pool of 100 nucleotides (nt) long DNA sequences. The 100 nt sequence contained a 40 nt long random sequence and constant regions in both ends for the primer binding: 5′-ACC CCT GCA GGA TCC TTT GCT GGT ACC-40×N TAA GAC CCC GGG AGA TCT GAC TAA TCG CTA-3′. This initial ssDNA library (0.5 pmol) was amplified by PCR. The PCR products were separated on agarose gel and purified directly from the gel or with the spin columns.

### 2.3.4. DNA Precipitation

### 2.3.4.1. Ethanol Precipitation

One tenth of 3 M Sodium acetate and three volumes of 95-100% Ethanol were added before the samples (100 μl) were centrifuged for 10 min at 13,000 rpm at 4° C. The supernatant was removed and the DNA pellet washed with 500 μl of 70% Ethanol (stored in −20° C.) and centrifuged for 10 min as described above. The supernatant was removed and the pellet air dried before the pellet was redissolved into TE-buffer (pH8.0).

### 2.3.4.2. Isopropanol Precipitation

Isopropanol (0.65 volumes) was added to the DNA solution (100 μl) and mixed well before the samples were centrifuged at 13,500 rpm at 4° C. for 15 min. The supernatant was removed and the DNA pellet washed with 500 μl of 70% Ethanol (room temperature) to remove the salt and isopropanol residues. The samples were centrifuged for 8 min at 13,500 rpm and the DNA pellet was air dried before redissolved into the TE-buffer (pH 8.0).

### 2.3.5. Selection of Aptamers Specific for Live Bacterial Cells—Filtration Method

The selection of DNA aptamers against killed Francisella tularensis bacteria was described by Vivekananda & Kiel (2006). The selection method based on filtration was followed with some modification. The double stranded DNA (dsDNA) library (2.3.3) was extracted and purified from the agarose gel and heated for 3 minutes at 94° C. with an equal volume (45 μl) of binding buffer (BBf) and cooled on ice to separate the strands. To exclude the filter binding single stranded DNA (ssDNA) sequences, the ssDNA samples with an equal volume of BBf (45 μl) were applied on the MultiScreen filter plates and drawn through the filter by using a vacuum manifold. The samples were washed three times with 50 μl of BBf and the flow through samples containing non-filter binding ssDNA sequences were collected and amplified by PCR. Ready-to-go PCR beads were used with 1 μl of template DNA (non-filter binding) and 25 μM of each primer PR1 and PR2. The amplification parameters were 5 min at 94° C. for initial denaturation, denaturation at 94° C. for 1 min, annealing at 62° C. for 1 min, and elongation at 72° C. for 1 min. The final elongation was at 72° C. for 10 minutes. The samples were separated on agarose gel (2.3.2) and purified with the PCR product purification kit.

Four colonies of bacterial cells from the Nutrient agar were suspended into PBS following a centrifugation for 10 min at 6000 g. The washing step was repeated twice and the bacterial pellet was resuspended into 500 μl of BBf. The non-filter binding PCR amplified pool of DNA in BBf (100 μl) was denatured as described above and mixed with the bacterial cell suspension (100 μl). The control samples without ssDNA library were performed in parallel. After a 60 min incubation at room temperature with a gentle rotation, samples were applied on a 96 well filter plate (100 μl each well) and drawn through the filter. The unbound sequences were washed three times with 50 μl of BBf. In order to elute the bound molecules, 100 μl of boiling Elution buffer was added to the samples and the aptamers were collected and precipitated with ethanol or isopropanol (2.3.4). To enrich the pool of aptamers, the samples were amplified by PCR under the conditions previously described. The selection process was repeated by using the enriched pool of aptamers in the following rounds of selection.

### 2.3.6. Selection of Aptamers Against Live Bacterial Cells—Centrifugation Method

The selection of aptamers against live bacterial cells was first described by Hamula et al. (2008). In this study their protocol was followed with some modifications. Fresh overnight cultures of bacterial cells were used in every round of selection. Cell suspension (1 ml) was washed three times by centrifuging at 3500 g for 5 min at 4° C. and resuspended in 500 μl of 1× binding buffer (BB). The dsDNA was denatured to ssDNA by heating at 94° C. for 5 minutes and then cooling on ice for 10 minutes. 100 μl of cell suspension and 25 μl of ssDNA library (PCR product) were mixed with BB containing 125 μg/ml tRNA and 0.005% BSA in order to reduce non-specific binding. Low DNA binding (LoBind) tubes were used to reduce the aptamers to bind the tube. The mixture was incubated at room temperature with gentle rotation for 45 min. Unbound aptamers were washed three times after each of the first seven incubations and five times on the eighth round of selection with 250 μl of BB containing 0.05% BSA by centrifuging the cells at 4000 g for 5 min at 4° C. and collecting the supernatant. Bacterial cells and DNA were separated and the aptamers-containing supernatant was collected. Tubes were replaced with new fresh tubes after the first and third washes in order to eliminate aptamers which may have bound non-specifically to the tube wall. After the washes the bacterial cells with bound aptamers were resuspended in 10 mM Tris-Cl (pH 8.5) and the cells were heated to 94° C. for 10 min to release the captured aptamers from the cells. The cells were centrifuged and the aptamers containing supernatant collected. The aptamer pool (supernatant) (1 μl) was amplified by PCR (2.3.1). The primers and other PCR parameters were the same as those used to produce the DNA library and the PCR amplification was repeated 20 cycles.

Counter selection was performed in order to eliminate aptamers that bind bacteria other than the one of interest. The counter selection was performed after selection round 5 and round 8. The protocol for counter selection was the same as that used when selecting specific aptamers except that the unbound DNA was collected and used as a new pool of aptamers. The number of PCR cycles was reduced to 15 cycles and the template concentration had to be lowered in order to obtain 100 bp PCR products. The template was diluted 1:30 and 1:40.

### 2.3.7. Labelled Aptamers

Aptamers were labelled with biotin (BIO) or fluorescence (FAM) by amplifying the aptamer pools by PCR (2.3.1) using 5′ biotinylated or 5′ FAM-labelled primers PR1 and PR2. All PCR amplification conditions remained the same. The PCR products were separated and visualised on agarose gel and the products were purified with the spin columns as described. Before the binding reaction, aptamers were strand separated by heating the aptamers at 94° C. for 10 min and cooling on ice immediately.

### 2.3.8. Detection of Aptamer Binding by Enzyme Linked Technique

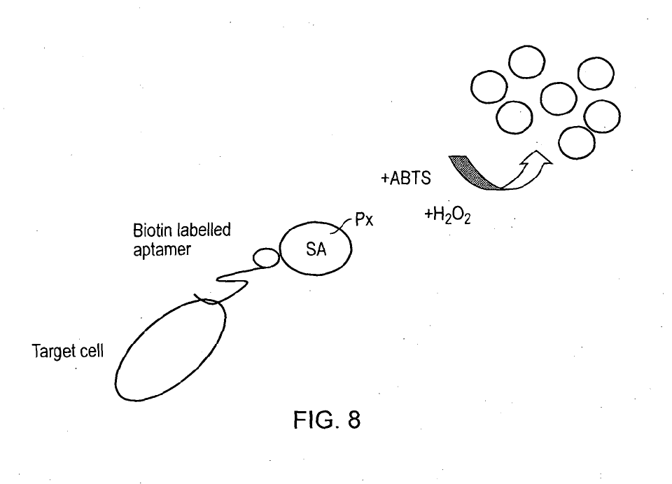

Fresh overnight bacterial culture was prepared as previously described (2.3.6) and biotin-labelled aptamers were produced (2.3.7). 100 μl of single stranded aptamer solution was added to an equal volume of bacterial suspension in BB and incubated for 45 min at room temperature with gentle rotation. Unbound aptamers were washed three times by centrifuging the cells at 3500 g at 4° C. for 5 min and resuspending in BB containing 0.05% of BSA. New fresh tubes were changed via resuspension after the incubation. The cells were resuspended in the PBS containing 0.1% BSA and 1 μg/ml peroxidase labelled Streptavidin and the samples were incubated for 45 min at room temperature in gentle rotation to allow streptavidin to bind to biotin. Unbound streptavidin was washed three times with PBS and fresh clean microcentrifuge tubes were changed after the first and third wash via resuspension. The ABTS substrate (1%) in 0.05M citrate buffer in presence of 0.3% H₂O₂ was added and after 40 min incubation the cells were centrifuged and the absorbance of the reaction mixture measured at 405 nm. Five different dilutions of aptamers (1:2, 1:4, 1:10, 1:20, 1:40) were tested in triplicate. The reaction is illustrated in FIG. 8.

### 2.3.9. Detection of Fluorescence Aptamers

### 2.3.9.1. FAM-Labelled Aptamer Pools Binding to the Bacterial Cell Surface

FAM-labelled aptamers were produced (2.3.7) and strand separated by heating the aptamer pool at 94° C. for 10 min and cooling immediately on ice. A bacterial suspension was prepared by centrifuging of fresh overnight grown culture (1.5 ml) at 3500 g for 5 min at 4° C. The bacterial pellet was then washed and resuspended in 500 μl of BB. The bacterial suspension (100 μl) was incubated with denatured single-stranded aptamers for 45 min at room temperature in gentle rotation in LoBind tubes followed by centrifugation of 3500 g for 5 min at 4′C. Unbound aptamers were washed three times with 250 μl BB to remove the unbound aptamers. Incubation time was optimised to 45 min.

### 2.3.9.2. Fluorescence Microscope

On a microscope slide 6 μl of bacterial cell suspension was added and the samples were viewed under the 60× (Nikon) or 100× (Olympus) magnification with the filter settings for green light (Excitation/emission maxima 520/495 nm) and normal visible light. The pictures were taken and analysed.

### 2.3.9.3. Fluorimetry

A fluorescence plate reader (495 nm, Em 520) was used to measure the fluorescence of the FAM-labelled aptamers with a sensitivity of 50. Washed bacterial cells with bound aptamers were resuspended in 100 μl of BB and the samples were applied to black 96-well plates.

### 2.3.10. FluoSpheres® Fluorescence Microspheres

The bacterial suspension and the aptamer binding reaction were performed as described for FAM-labelled aptamers (2.3.10.1), except biotin-labelled aptamers were used. Biotin-labelled aptamers were produced (2.3.7) and the aptamers were strand separated by heating at 94° C. for 10 min and cooling immediately on ice. The bacterial cells with biotin labelled aptamers on the surface were washed once with BB and resuspended into PBS with 1% fluorescence microspheres. The samples were incubated at room temperature for 45 min with a gentle rotation to let the fluoSpheres to bind to the biotin labelled aptamers. 1% BSA was used to block the non-specific binding sites. After the incubation, samples were washed twice with PBS and the pellet was resuspended into 30 μl PBS. 6 μl of this suspension was placed on a microscope slide and the samples were viewed under×100 magnification with green light (Excitation 488 nm) and normal visible light. The pictures were taken and analysed.

### 2.3.11. Live/Dead® BacLight™ Staining

Bacterial cells were stained with Live/Dead BacLight kit to distinguish the aptamer binding between the live and dead bacterial cells. The staining is based on CYTO-9 green-fluorescent nucleic acid stain that stains all the bacteria and the Propidium iodide red-fluorescent nucleic acid stain that only stains the bacteria with damaged membranes. FluoSpheres (2.3.10.2) were used to visualise the aptamer binding. Bacterial cells with biotin aptamers and FluoSpheres bound to them were resuspended into 0.85% NaCl solution. An equal volume of staining solution (1 volume CYTO-9, 4 volumes Propidium iodide) was added to the suspension and incubated for 15 min at room temperature. 6 μl of bacterial cell suspension was added on a microscope slide and the samples were viewed under the 100× magnification with the filter settings for green (Excitation/emission maxima 480/500 nm) and for red fluorescence (Excitation/emission maxima 490/635 nm). The pictures were taken and analysed.

### 2.3.12. Identification of Aptamer Sequences

### 2.3.12.1. Cloning of Aptamers

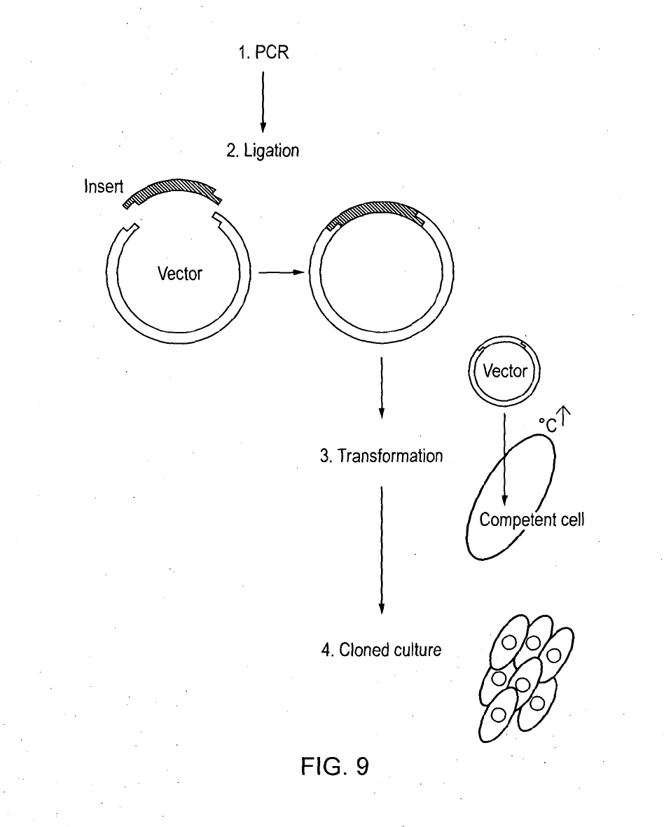

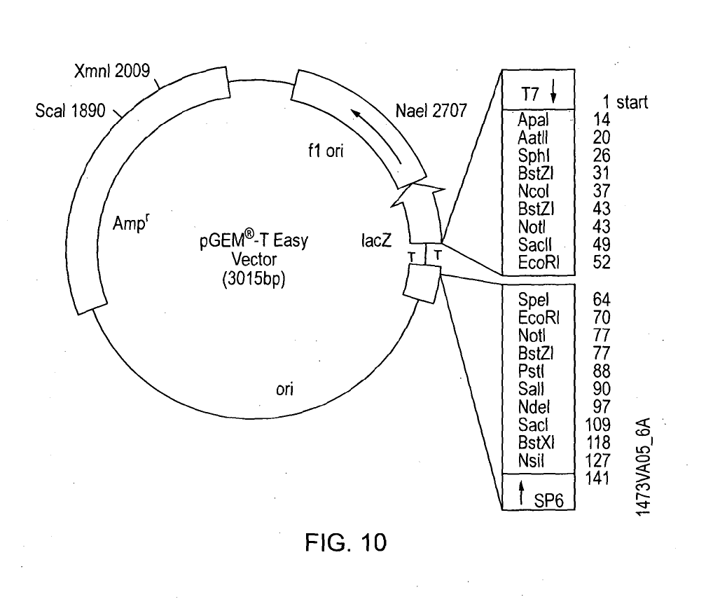

The pGEM-T Easy Vector system was used for cloning the aptamer pools. The cloning was performed by following the instructions manual (Promega Technical Manual). In FIG. 9 is a schematic presentation of the main cloning steps. First the aptamers are PCR amplified (1. PCR) and the products purified with the spin columns. Purified aptamers are ligated to pGEM-T Easy vector by incubating in a rapid ligation buffer for an hour at room temperature (2. Ligation). The linearised vector (FIG. 10) is designed with a single 3′-terminal thymidine (T) overhang at both ends. This improves the efficiency of ligation of PCR products by preventing recircularisation of the vector and providing a compatible overhang for PCR products generated by Taq-polymerases (Promega Technical Manual). The insert vectors are then transformed into the competent cells (FIG. 9, 3.Transformation) and the cells are then incubated in order to enrich the amount of the bacterial cells that have the insert vector inside (4. Cloned culture).

The aptamer pools 9 were PCR amplified (2.3.1) before they were ligated into the vector. Reaction components and amounts for the ligation reaction are presented in the Table below. Positive control with control insert DNA and background control without an insert were performed. The ligation reactions (vectors with the inserts) were first incubated on ice with the competent cells (thawed on ice for 5 min) for 20 minutes before transformed into the JM109 competent cells.

Reaction components for ligation.

| | Standard | | Positive |
|---|---|---|---|
| Reaction component | reaction | Background | control |
| Ligation buffer | 5 μl | 5 μl | 5 μl |
| pGEM-T Easy vector | 1 μl | 1 μl | 1 μl |
| PCR product | X μl | — | — |
| Control insert DNA | — | 2 μl | — |
| Ligase | 1 μl | 1 μl | 1 μl |
| H₂O to a final volume of | 10 μl | 10 μl | 10 μl |

The transformation of the vectors into the bacterial cells was done by heating the samples for 50 sec at 42° C. and cooled down on ice for 2 minutes. Transformation efficiency was estimated by using the uncut plasmid (0.1 ng). The competent cells were then incubated in SOC medium (950 μl) for 1.5 h at 37° C. with 150 rpm rotation followed by the plating of the samples (100 μl) on the selective indicator plates. The plates were incubated over night at 37° C.

### 2.3.12.2. Analysing the Positive Colonies—Colour Selection

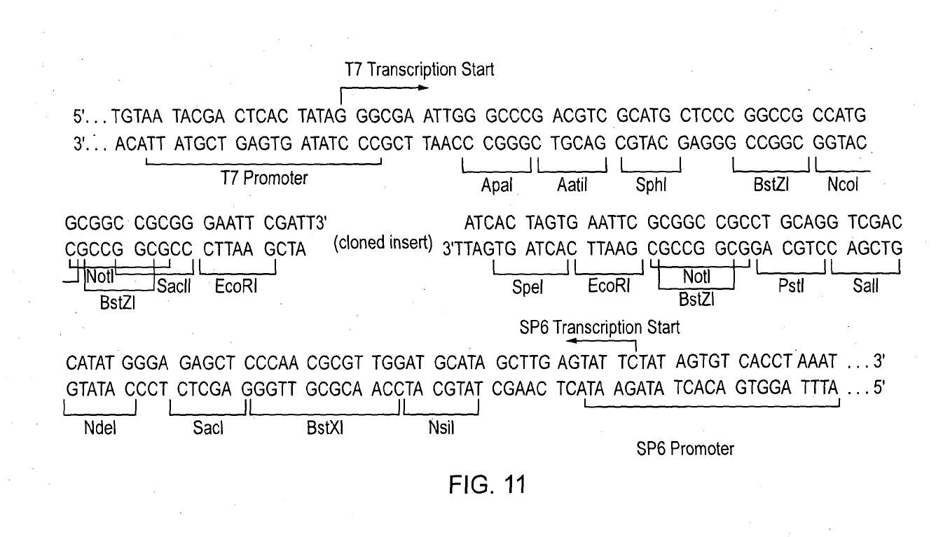

pGEM-T Easy vector has a multiple cloning region (FIG. 11) within the α-peptide coding region of the enzyme β-galactosidase (Promega Technical manual). The DNA insert deactivates the α-peptide and makes the colour screening of the recombinants possible on the indicator plates. Positive colonies were white on the plates and negative colonies were blue. Positive colonies were selected from the plates and transferred to a new selective plate. After an overnight incubation the clones were analysed by PCR and restriction analysis.

### 2.3.12.3. Analysing the Positive Colonies—PCR Analysis

For PCR analysis, PCR Supermix HiFi was used with 25 μM of both primers (PR1 and PR2) and one colony on the plate was used as a template. The amplification parameters were 10 min at 94° C. for initial denaturation, denaturation at 94° C. for 45 s, annealing at 62° C. for 45 s, and elongation at 72° C. for 45 s. The final elongation was 10 min at 72° C. The PCR cycles denaturation, annealing, and elongation were repeated 20 times. PCR products were separated on agarose gel and the pictures were taken (2.3.2).

### 2.3.12.4. Plasmid Extraction

One positive colony from the indicator plate was incubated in 5 ml LB-broth (ampicillin 50 μg/ml). The plasmid vector was extracted from a 3 ml of overnight culture by using a plasmid extraction kit. The pure plasmid was then used for restriction analysis and sequencing.

### 2.3.12.5. Analysing the Positive Colonies—Restriction Analysis

EcoRI was used as a restriction enzyme to digest the insert from the plasmid. The restriction map for pGEM-T Easy vector is shown in FIG. 10. Enzyme EcoRI (1 μl) was added to 14 μl of sterile water with 2 μl 10× Buffer (provided with the enzyme) and 3 μl purified plasmid. The reaction was incubated at 37° C. water bath for 1 h. The restriction products (9 μl each) were separated on an agarose gel (2.3.2).

### 2.3.12.6. Sequencing of Cloned Vector

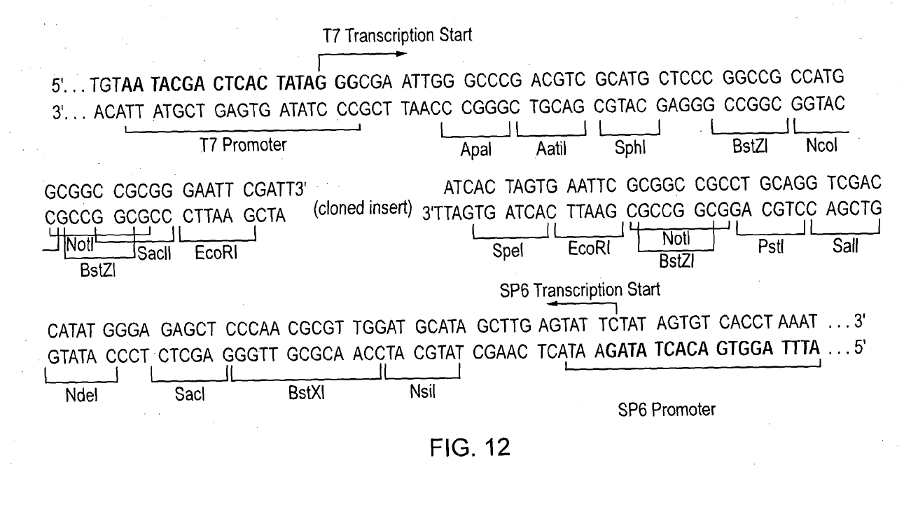

The aptamers were cloned by using pGEM-T Easy Vector system and the plasmid DNA was purified with Quick Plasmid Miniprep Kit. The plasmid DNA (30 μl) samples were sent in a microcentrifuge tube to sequencing. The sequencing primers (T7 and SP6r) were designed to match to vector's multiple cloning sites. The primer binding sites are marked in FIG. 12 in red. The first forward sequencing primer (T7) was selected from the sequencing suppliers Services à la Carte (list of standard primers). The reverse primer (SP6r) was synthesised for sequencing.

### 2.3.12.7. Aptamer Sequence Analysis

The 100 nt long aptamer sequences were identified from the vector sequence by comparing the primers PR1 and PR2 sequences to the vector sequence. This 100 nt aptamer sequence was analysed by UNAFold program and the length of the aptamers was reduced to 35-70 nucleotides. Aptamers were synthesised with a fluorescence FAM-label and the binding was tested (2.3.9).

The aptamer binding to its target is dependent on its secondary structure. The possible secondary structures of the aptamer sequences were analysed using OligoAnalyzer 3.1 UNAFold program. The sequence was given to the program for analysis of sodium concentration being 100 mM and magnesium concentration 1 mM as in binding buffer (BB). The temperature was set to 25° C. The most common secondary structure given was used as a template to reduce the length of the nucleotide sequence of the aptamers from 100 bases to 35 to 70 bases. The analysis of these aptamers was done with the UNAFold.

### Example 3

### Selection of Specific Aptamers Against Non-Pathogenic Escherichia coli K12

### 3.1. Introduction

Aptamers are single-stranded DNA or RNA ligands that can be selected to bind to proteins but also smaller molecules such as organic dyes (Ellington & Szostak, 1990) as well as prions (Iqbal et al., 2000), bacterial cells (Hamula et al., 2008) and viruses (Symensma et al., 1996). Binding of the aptamers to their target mainly encompasses all types of non-covalent binding except normal standard nucleic acid bond formation (Watson-Crick base pairing).

This work was focused on optimising the techniques necessary for the routine preparation of aptamers against live bacterial cells. Aptamers were selected from a randomly created DNA library using a selective evolution technique against live bacterial cells of non-pathogenic Escherichia coli K12. The development of selection techniques began with a technique based on filtering. Filter membranes were used to separate the non-target binding molecules from the ones binding the target.

Because of the difficulties of the aptamer elution procedure of the filtering method, a new technique, that is easier and faster to perform, was introduced. The new technique, centrifugation method, is based on different weights and sizes of the molecules. Nine rounds of selection were performed and counter selection was used to deselect aptamers that were binding to bacterial cells other than E. coli K12. The counter bacteria used were Lactobacillus bulgaricus after selection round 5 and Bacillus subtilis and Salmonella typhimurium after selection round 8. The aptamer pool 9 was biotin labelled and the binding of the aptamers was tested with an enzyme linked technique.

### 3.2. Methods

### 3.2.1. DNA Library Production

A random DNA library was produced (2.3.3). The PCR cycle (denaturation-annealing-elongation) was initially repeated over 30 cycles with 1 min reaction times resulting in the formation of non-specific products. Primers were analysed with an Oligoanalyzer to see the primer-dimers that can possibly be formed. The reaction times, temperatures, template concentration and the number of PCR cycles were optimised. The PCR products were separated on agarose gel (2.3.2) and the pictures were taken. The DNA library was purified from gels with a gel extraction kit.

3.2.2. Selection of E. coli K12 Specific Aptamers

The aptamers were selected from a random DNA library to bind specifically to non-pathogenic strain E. coli K12. Filtration selection (2.3.5) was first used to select the aptamers but was found to be overly complicated. The elution of the bound aptamers was difficult, as boiling elution buffer with high urea concentration was needed. It was impossible to add the buffer that was boiling on the filter. The other major problem occurred when the elution buffer was boiled for too long and some of the water was evaporated. This resulted in a precipitation of the urea on a filter making the aptamers impossible to elute. The EDTA in the elution buffer keeps the DNA more stable when stored but the DNA had to be precipitated, purified and redissolved in another buffer for next steps of the selection. The centrifugation method (2.3.6), which did not suffer from these constraints, was developed and used for aptamer selection.

The first 5 aptamer pools were selected (2.3.6) and the pools were amplified by PCR (2.3.1) using 20 reaction cycles. The first counter selection was performed after the fifth round of selection where L. bulgaricus was used as a counter bacterium. The samples were collected and amplified by PCR. After the counter selection the PCR was optimised. The template was diluted to 1:30 and the number of amplification cycles was reduced to 15 cycles. The template control was performed to see if the addition of a template to the reaction appears as a band on an agarose gel.

Aptamer pool 5, that has gone through the counter selection, was used as an aptamer pool for selection round 6. Samples were amplified by PCR with 15 PCR cycles. Selection round 7 was performed as normal and both replicates were amplified by PCR twice in two separate tubes due the low PCR product yield after the previous selection round. Those two PCR products were extracted and mixed together and aptamer pool 8 was selected from this pool of aptamers in two replicates. The number of PCR cycles was increased to 20 cycles as 15 cycles did not result in a PCR product that could have been seen on an agarose gel. This increase of the PCR cycles resulted in a higher number of aptamer copies and therefor the PCR product can be visualised on an agarose gel. Two replicates were amplified in two separate reaction tubes and mixed together.

The second counter selection was done by selecting the aptamers that are not binding to B. subtilis or S. typhimurium. Aptamer pool 8 was used for the selection and the non-binding samples were collected and amplified by PCR. The template for the PCR was diluted 1:40 and 15 rounds of PCR was used.

The ninth pool of aptamers was selected from the pool 8. Pool 9 was PCR amplifies and separated on polyacrylamide gel (2.3.2). Polyacrylamide gel was used for these samples in order to see which one, agarose or polyacryamide gel, is more suitable for separating the aptame PCR products.

3.2.3. Biotin Labelled E. coli K12 Binding Aptamers

Aptamer pool 9 specific for E. coli was biotin-labelled by PCR using 5′ biotin-labelled primers (2.3.7).

3.2.4. Detection of E. coli K12 Binding Aptamers by Enzyme Linked Technique

The biotin-labelled aptamer pool 9 selected to bind E. coli K12 was incubated with bacterial culture (2.3.8). Bacterial cells with biotin-labelled aptamers bound to them were incubated with streptavidin peroxidase following the addition of ABTS and H₂O₂. The colour change was detected by measuring the absorbance of the samples at 405 nm. Five different dilutions of aptamers (1:2, 1:4, 1:10, 1:20, 1:40) were tested in triplicate. The results were analysed with the analysis of variance (ANOVA).

### 3.3. Results and Discussion

### 3.3.1. DNA Library Production

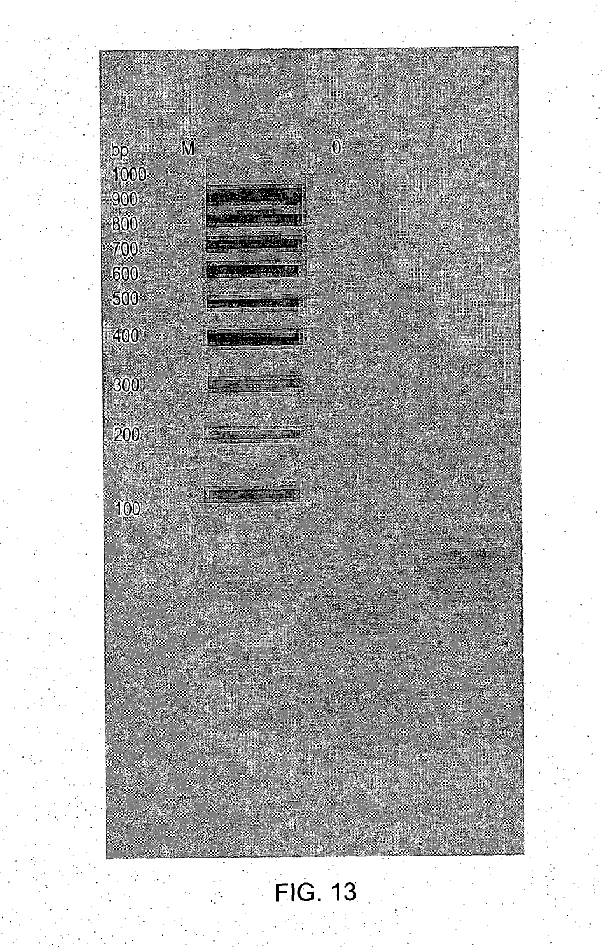

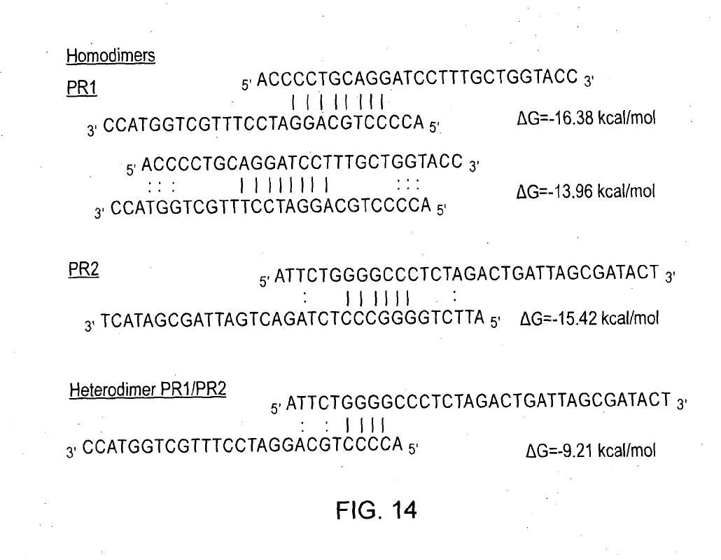

The random DNA library was produced by PCR and the samples were separated on agarose gel. When the PCR amplification reaction was repeated 30 cycles, and the reaction (denaturation, annealing and elongation) times were 1 min, non-specific products were observed. In these conditions non-specific PCR products were appearing on agarose gel pictures as extra bands (FIG. 13). In lane 0 is a PCR control, where only PCR primers PR1 and PR2 are added, products smaller than 100 bp and a faint 100 bp band can be seen. In lane 1 where the template DNA is added, two bands can unexpectedly be seen. These extra bands can be caused by the dimerisation of the primers, for example, formation of self-dimers or hetero-dimers. In FIG. 14 the strongest primer-dimers likely to be formed with the primers PR1 and PR2 are presented. Nevertheless, the strongest homo-dimer having a ΔG—16.38 kcal/mol is not as strong as the bonding between the primers and their complementary target sequences (ΔG for PR1—55.47 kcal/mol and for PR2—62.09 kcal/mol).

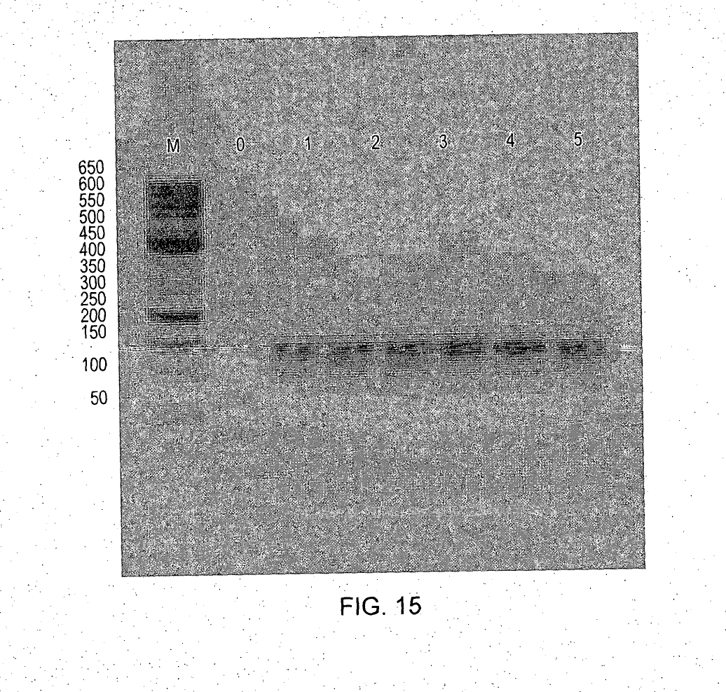

PCR products without non-specific products were achieved when the number of the PCR cycles was reduced to 15 or 20 and the reaction times were reduced from 1 min to 45 s. PCR control sample appears to be clear and non-specific bands cannot be observed on gel pictures. The DNA library was produced by using these conditions and the agarose gel of the library is shown in FIG. 15. The samples (DNA library) are on gel in lanes 1-6 and can be seen as thick bands. The PCR products were expected to be 100 bp in size because the DNA library size was 100 nucleotides. The bands on the gel seem to be 140 bp instead of 100 bp. It has been found that the pre-stained agarose gels might affect the mobility of DNA on gel and especially small DNA fragments might be affected (Miller et al. 1999) making it difficult to estimate the size of the PCR product on agarose gel. Therefore the actual product size is very likely to be 100 bp. It is possible, when the PCR yield is large, that the samples do not move on the gel as fast as the samples with containing less DNA. This could have been confirmed by adding less PCR product on the gel. Two different template concentrations were used in PCR reaction. In lanes 1, 2 and 3, 0.1 pmol template was added and in lanes 4, 5 and 6, 0.5 pmol template was added. It can be seen that this concentration change did not affect to the actual PCR yield. In lane 0 is the PCR control sample where no template DNA was added. The faint band, smaller than 50 bp, is the primer dimer.

3.3.2. Selection of E. coli K12 Specific Aptamers

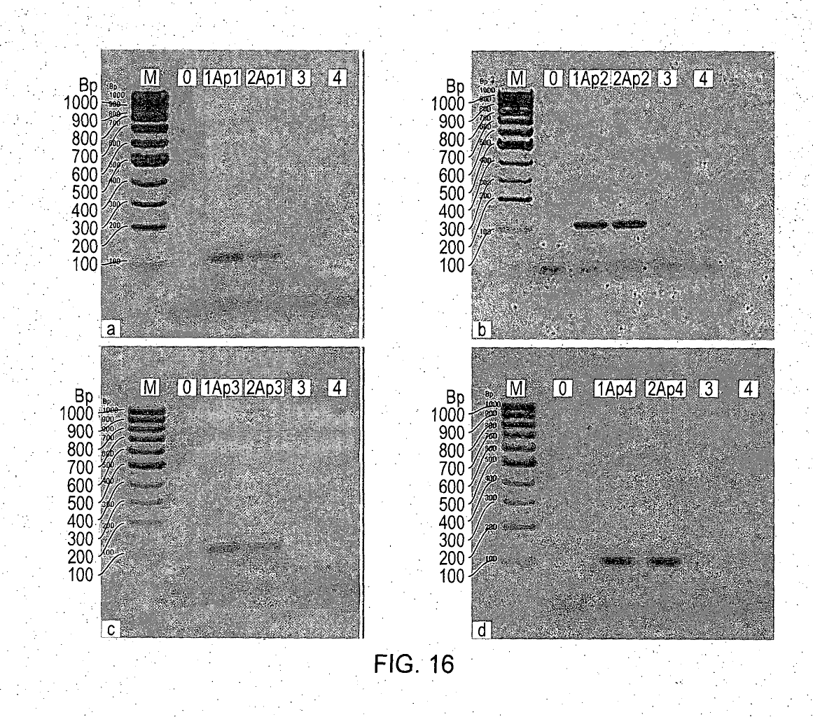

Aptamers were selected to bind non-pathogenic strain E. coli K12 by using a centrifugation method first described by Hamula et al. (2008) with some modifications. Nine rounds of selection and counter selection after selection round 5 and 8, were performed. Agarose gels after the selection round 1, 2, 3 and 4 are presented in FIG. 16. The aptamer pools can be seen in the gel images in lanes 1 and 2 as 100 bp or slightly bigger bands. No amplification can be seen in DNA control samples where no bacterial cells were added (lanes 3), as expected. This is because the aptamers have been washed off from the samples as there are no binding sites for them in the solution. This also shows the aptamers are not binding anything else such as the tube wall. Also, the bacterial control samples in lanes 4 are clear. This shows E. coli K12 has no DNA sequence for the PCR primers used in this experiment to bind and therefore cannot be amplified in PCR. The PCR control samples in lanes 0 have no 100 bp bands, only a faint band (smaller than 50 bp) that is a primer dimer, as expected.

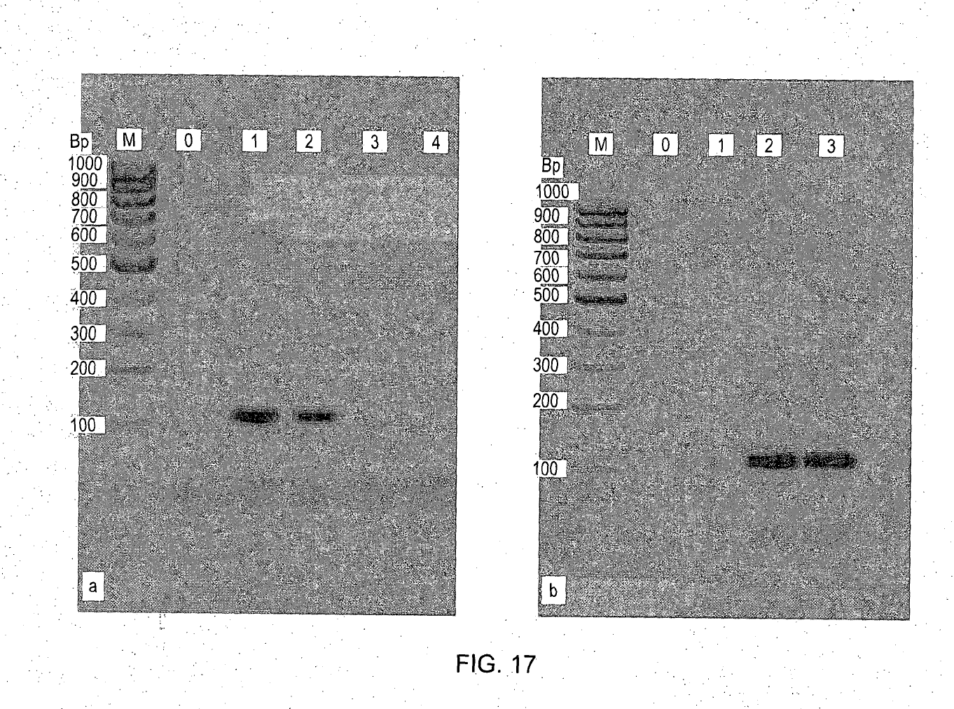

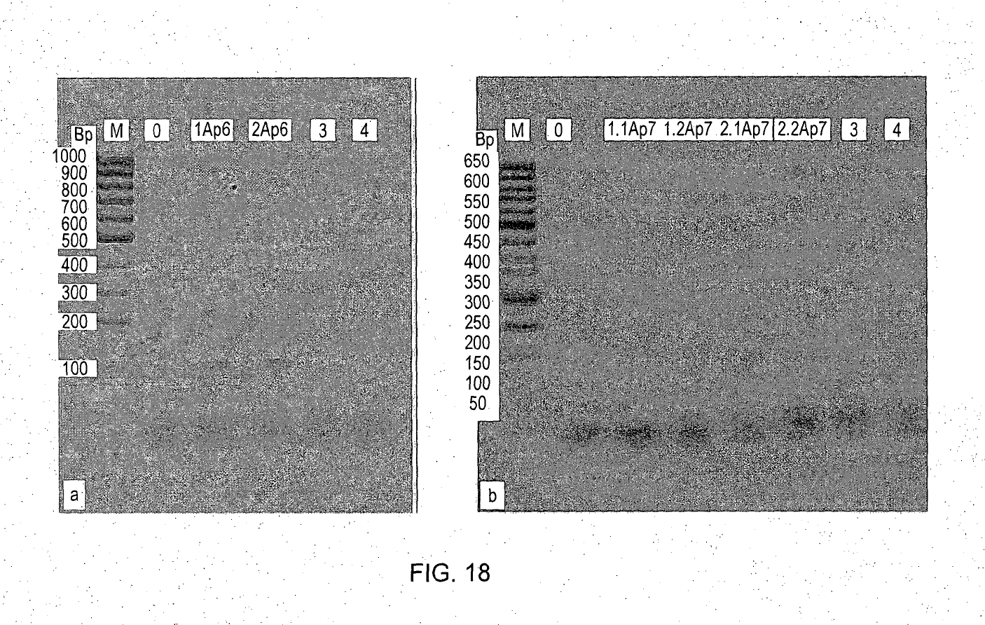

After five rounds of selection, the counter selection was performed by using L. bulgaricus as a counter bacterium. The agarose gel pictures are presented in FIG. 17. The PCR product of aptamer pool 5 is on gel a (FIG. 17a) in lanes 1 and 2 and the aptamer pool 5 after the counter selection is on gel b (FIG. 17b) in lanes 1 and 2. The counter selection products are the aptamers that are binding E. coli K12 but not L. bulgaricus. A template control in lane 1 on gel b (FIG. 17b) was performed to see if the addition of a template can be seen on an agarose gel. As expected, no 100 bp template band can be seen and this indicates that the bands seen on gel images are PCR amplification products. Lanes 0 on the gels are PCR control samples where the primers were added with no template DNA. All these control samples are clear as expected. The faint bands around 50 bp are the primers.

Agarose gel pictures of aptamer pools 6 and 7 are presented in FIG. 18. The aptamer pool 6 has faint bands on the gel a (FIG. 18a). Because of the small yield of the PCR product 6, two aptamer pool 7 samples were amplified in two replicates. Samples 1.1 and 1.2, and samples 2.1 and 2.2 on gel b (FIG. 18b) were mixed together after they were purified in order to achieve an aptamer pool with more aptamers. On lanes 0, where the PCR control samples are, no amplification can be seen because no template DNA was added. No amplification can be seen on bacterial control samples (lanes 3) or in DNA control samples (lanes 4), as expected. The bacterial control sample only contains bacterial cells and the DNA control samples the DNA aptamers that have been washed off because there were no binding sites for the aptamers in the mixture. The faint band, smaller than 50 bp, contains the primers.

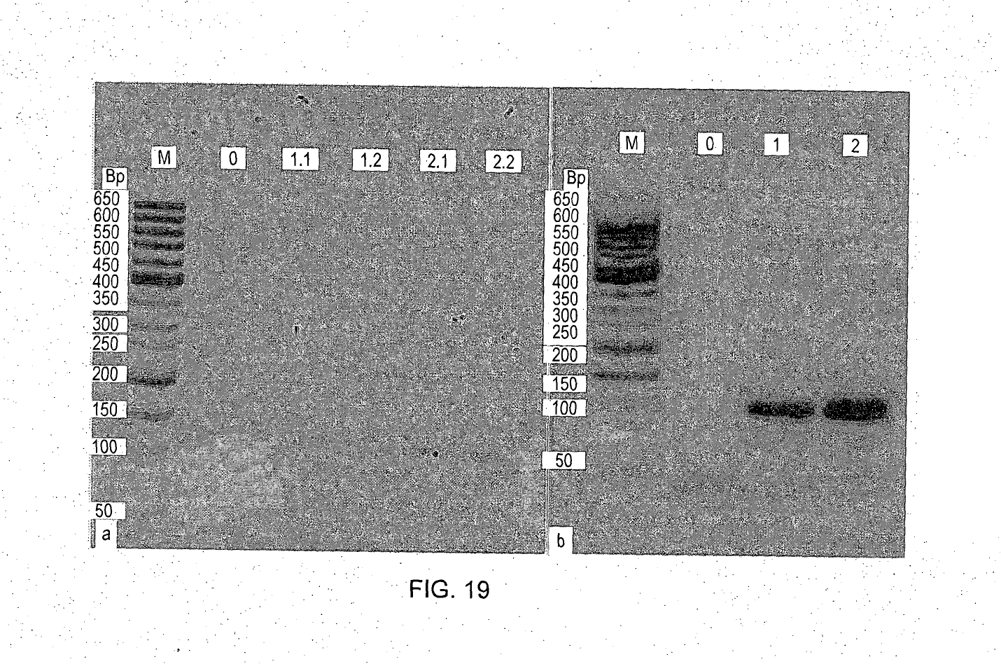

The agarose gel pictures of the aptamer pool 8 before and after the counter selection are presented in FIG. 19. On gel a (FIG. 19a), it can be seen that the aptamer pool samples (100 bp) are faint and therefore the samples have been amplified in two replicates (1.1, 1.2, 2.1 and 2.2). The counter selection was performed with B. subtilis and S. Typhimurium and the PCR products separated on agarose gel can be seen on gel b (FIG. 19b) in lanes 1 and 2. It can be seen that the intensity of the PCR products after the counter selection is much higher than before. That is because most of the aptamers in the pool are not binding to counter bacteria and therefore more template has been added to the amplification reaction. PCR control samples where only the primers have been added are on lanes 0 and no amplification products can be seen on these lanes, as expected. The faint bands smaller than 50 bp are the primers.

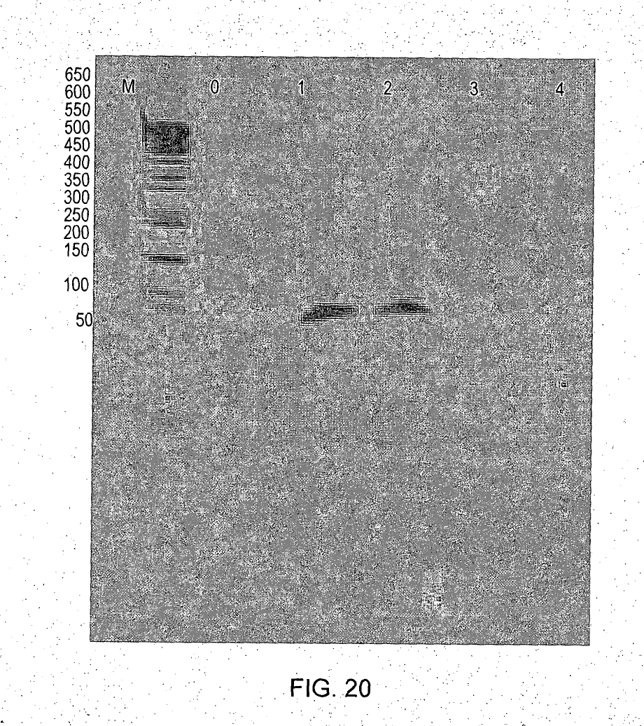

The aptamer pool 9 was selected and the PCR products were separated on polyacrylamide gel. Polyacrylamide gel was used to see if it is suitable for separating aptamer samples. The gel picture of the aptamer pool 9 and the control samples are presented in FIG. 20. Two bands can be seen on the gel in lanes 1 and 2 around 100 bp. These samples are aptamer pool 9 in two replicates. It can be seen that the molecular weight marker (lane M) has unusual bands when comparing the bands to the other gel images above. It seems that the ladder used in this experiment is not suitable for polyacrylamide gels. The bacterial control sample in lane 3 and the DNA control sample in lane 4 are clear, as expected. The bacterial control sample only contains bacterial cells and the DNA control sample only contains the DNA aptamers that have been washed off because there were no binding sites for the aptamers in the mixture. PCR control sample, where only the primers have been added, is on lane 0. No amplification can be detected, as expected. The faint bands at the bottom of the image are the primers. The polyacrylamide gel can be used for separating the samples, but agarose gels were used in further experiments because of their easiness.

3.3.3. Biotin Labelled E. coli K12 Binding Aptamers

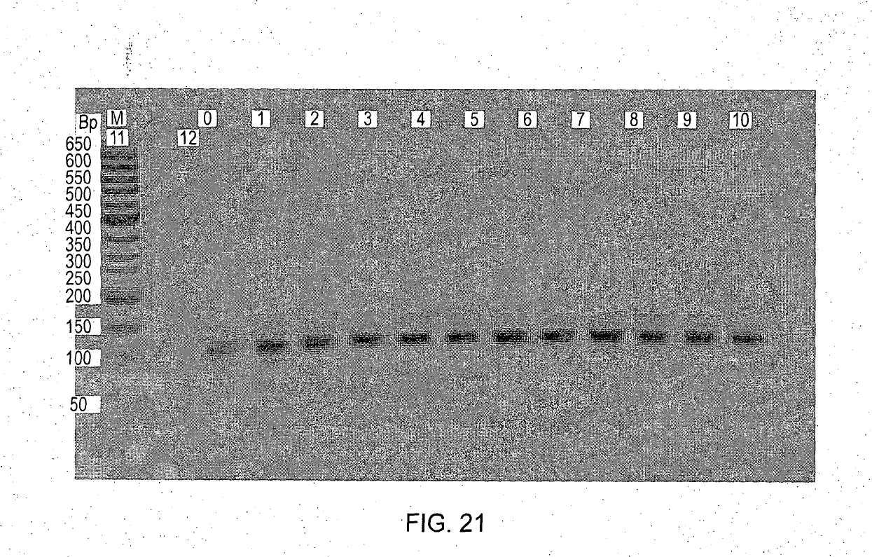

The biotin-labelled aptamer pool 9 was produced by PCR. The labelling was done by using biotinylated primers. The PCR products were separated on agarose gel to see the size of the products. In FIG. 21 biotin-labelled aptamer pool 9 selected to bind E. coli K12 is presented. Aptamer pools are the 100 bp bands in lanes 1-12. In lane 0 the PCR control sample where no template DNA was added is clear, as expected. Samples were purified with spin columns and used for the binding reaction.

### Example 4

### Development of Fluorescence Based Detection Method for Escherichia Coli K12 Binding Aptamers

### 4.1 Introduction

The method for selecting aptamers against live bacterial cells was developed. The specific aptamers were selected to bind to non-pathogenic E. coli K12 by using a method based on centrifugation (3.2.2). The binding of these aptamers to their target was demonstrated with an enzyme linked method (3.3.4), where the aptamers were first labelled with biotin. As the strong binding between streptavidin and biotin is well known, peroxidase-labelled streptavidin was attached to biotin. Addition of a substrate led to a colour change when reacting with the peroxidase. This colour change correlates to the amount of aptamers bound to the bacterial cell surface. This method demonstrates the aptamer binding, but is time consuming due the number of washes. Also too many bacterial cells were washed off during the washes leading to variable results when the method was repeated.

In this study aptamers were labelled with a fluorescent (FAM) label. Fluorescence based detection methods for aptamer binding were developed, the binding properties of the aptamers characterised and the specificity tested. As E. coli K12 is a rather easy bacterium to work with, an alternative method to detect aptamer binding to smaller bacterial cells was tested. This method is based on fluorescent-labelled streptavidin beads that can bind to biotin labelled-aptamers and can possibly be used to test the aptamer binding to smaller and faster moving bacterial cells such as Salmonella. This study demonstrates the E. coli K12 aptamers binds specifically to its target.

### 4.2 Methods

4.2.1 Development of Fluorescence Detection Method for E. coli K12 Binding Aptamers

### 4.2.1.1. FAM-Labelled Aptamers

Aptamers were selected to bind to E. coli K12 (3.2.2). The aptamer pools used in the experiments were labelled with 5′ fluorescence (FAM) label (2.3.7) and by detecting the label the aptamers will be detected. For all the experiments, aptamer pools were produced by PCR using the aptamer pool 9 as a template. More template was produced by amplifying the aptamer pool 9 PCR product in dilution 1:40 (Nested template) by PCR. The PCR amplified aptamer pools were separated on agarose gels and the images were taken followed by a purification of the product by spin columns. The PCR product concentration was measured using a spectrophotometer to measure the absorbance of the samples at a wavelength 460 nm.

### 4.2.1.2 Fluorimetry

Fluorescence values of the E. coli K12 bacterial cells when FAM-labelled aptamers were bound to them were measured to detect the aptamer binding. E. coli K12 binding aptamers with FAM-labels were incubated with bacterial cells (2.3.9.1) at three different concentrations (10 pmol, 20 pmol and 30 pmol). Samples were incubated at room temperature for one hour and the samples were washed with binding buffer (BB). The fluorescence of the samples was measured with a fluorescence plate reader (2.3.9.3) and the results were analysed with ANOVA.

### 4.2.1.3 Fluorescence Microscope

The fluorescence microscope was used to visualise the fluorescent labelled aptamers bound to bacterial cell surface. E. coli K12 binding aptamers with FAM-labels were incubated with overnight grown E. coli K12 culture (2.3.9.1) and the samples were visualised under a fluorescence microscope (2.3.9.2). Two different concentrations (10 pmol and 50 pmol) were used and a control with no aptamers. The images were taken from six random fields with green (495 nm) and visible light and the fluorescent labelled bacterial cells were counted from the images. Results were analysed with a statistic test the analysis of variance (ANOVA).

### 4.2.2 Optimal Binding Time of the Aptamers

FAM-labelled aptamer pool 9 specific for E. coli K12 was used to measure the optimal binding time of the aptamers. The binding reaction (2.3.9.1) was performed with an aptamer pool 9 (10 μl, approximately 6 pmol) and 90 μl of an overnight culture. The samples were incubated at room temperature in triplicate for 0, 15, 30, 45, 60, and 75 minutes and the fluorescence values were measured with a fluorescence plate reader (2.3.9.3). The samples were washed three times with 200 μl of BB and the fluorescence was measured from the washes in order to see how many washes were needed to wash off the nonbinding aptamers. The fluorescence results were analysed with the ANOVA.

### 4.2.3. Binding of the Aptamer Pool 3, 5, 7 and 9

Binding of the aptamer pools was tested in order to see their binding capacity. Aptamer pools were collected after each round of aptamer selection (3.2.2) and the FAM-labelled aptamer pools were produced by PCR. Binding properties of the aptamer pools 3, 5, 7 and 9 were tested by incubating the aptamers with bacterial cells (2.3.9.1) and measuring the fluorescence of the samples. Aptamers were incubated in 50 μl of E. coli K12 suspension with three aptamer concentration (10 pmol, 15 pmol and 25 pmol) for aptamer pool 5 and 9. Due the small number of aptamers (PCR product), only two different aptamer concentrations (10 pmol and 15 pmol) were used for the analysis of aptamer pool 3 and 7. After 45 minutes incubation at room temperature the bacterial cells were washed and the fluorescence of the samples was measured with a fluorescence plate reader (2.3.9.3).

4.2.4 Specificity of the E. coli K12 Binding Aptamers

The specificity of the aptamers was tested in order to see if the E. coli K12 binding aptamers are specific to E. coli K12 or if they bind to other bacteria too. FAM-labelled aptamer pool was incubated with different bacterial cells. Two different types of experiments were performed. The samples were visualised under a fluorescence microscope and the fluorimetry experiments were performed. The aptamers were also tested to see if E. coli K12 could be detected from a mixture of bacterial cells.

4.2.4.1 Binding of the Aptamers to E. coli B, B. subtilis and S. aureus

The E. coli K12 specific aptamer pool 9 was tested with E. coli B, B. subtilis and S. aureus. E. coli K12 was used as a positive control. Bacterial suspensions were prepared (2.3.9.1) and 50 μl of this suspension was incubated with three different amounts of aptamers. E. coli K12 was incubated with 10 pmol, 20 pmol and 30 pmol of aptamers and the other strains (E. coli B, B. subtilis and S. aureus) with 5 pmol, 20 pmol and 30 pmol of aptamers. 5 pmol of aptamers were used instead of 10 pmol, because not enough aptamers were produced (small PCR yield). A negative control sample, where no aptamers were added, was made for all different bacteria samples. The fluorescence was measured from all of the samples by a plate reader (2.3.9.3). The microscope images were taken (2.3.9.2) from the 20 pmol samples (E. coli K12, E. coli B and S. aureus) with a green fluorescence light and visible light from five random fields and the fluorescent labelled bacterial cells were counted. The results were analysed with the ANOVA.

4.2.4.2 Detection of E. coli K12 from a Bacterial Mixture with FAM-Labelled Aptamers

The aptamer pool nine was tested to see if the aptamers are able to detect the E. coli K12 bacterial cells from a mixture of different bacterial cells. The bacterial mixture containing equal amounts of E. coli K12, E. coli B and S. aureus was prepared. Each bacterial suspension was prepared as described (2.3.9.1) and each suspension was mixed together to a total sample volume of 100 μl. The control samples were prepared by adding a third of bacterial suspension and topped up to 100 μl with BB. The aptamers (20 pmol) were incubated with the bacterial cell suspension followed by the washes. The fluorescence was measured by a plate reader (2.3.9.3).

4.2.4.3 Binding of the Aptamers to L. acidophilus

L. acidophilus is a common bacterium found in dairy products such as yoghurt and was therefore chosen to be one of the strains to be tested. The specificity experiment (4.2.4.1) was performed by incubating 20 pmol of FAM-labelled aptamers with a L. acidophilus strain. E. coli K12 was used as a positive control. The negative control samples, with no added aptamers, were made for both strains. The fluorescence values were measured by a fluorescence plate reader (2.3.9.3).

### 4.2.5 Fluorescence Microspheres

A method to visualise the biotin labelled aptamers (2.3.7) binding with fluorescent labelled NeutrAvidin microspheres (FluoSpheres) was developed (2.3.10). The method was developed in order to detect aptamers binding to some other bacterial cells that are smaller than E. coli K12 and therefore difficult to see under the fluorescence microscope using FAM-labelled aptamers. The FluoSpheres are small fluorescent NeutrAvidin spheres, and therefore have a high affinity to bind to biotin, as stated before for streptavidin (2.3.8). Biotin-labelled aptamers (50 μl) (eight PCR products in 100 μl spin column elution buffer) were incubated with E. coli K12 bacterial cells. The samples were washed and the FluoSpheres were added and incubated (2.3.10). Non-binding FluoSpheres were washed and the samples were visualised under a fluorescence microscope with a green fluorescence and visible light.

4.2.6 Aptamer Detection of Live and Dead E. coli K12 Bacteria Cells

The Live/Dead BacLight staining was introduced to see if the E. coli K12 specific aptamers are binding to live or dead bacterial cells (2.3.11). The FAM-labels of the aptamers were difficult to see on the microscope images because of the brighter fluorescence of the Live/Dead BacLight staining. Instead of using the FAM-labelled aptamers the FluoSpheres were used to detect the bound aptamers. The biotin labelled aptamer pool was first incubated with E. coli K12 as previously described (4.2.5). Once the aptamers and the fluorescence microspheres were bound to the bacterial cell surface the Live/Dead staining was performed (2.3.11). The samples were visualised by a fluorescence microscope and the images were taken.

### 4.3 Results and Discussion

4.3.1 Development of Fluorescence Detection Method for E. coli K12 Binding Aptamers

### 4.3.1.1 FAM-Labelled Aptamers

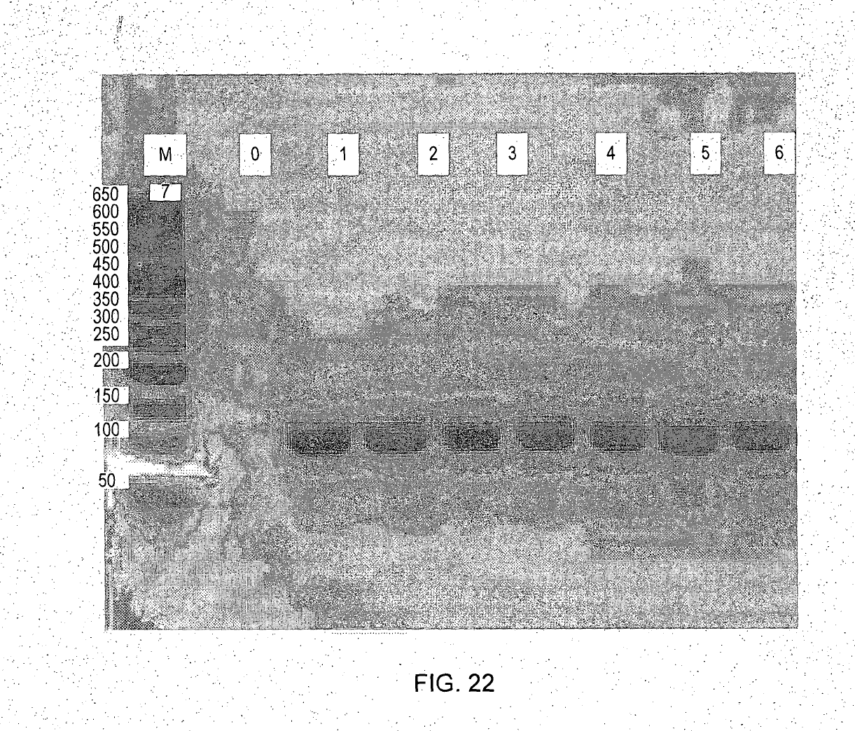

Aptamers were produced for all different experiments with FAM-labelled primers by PCR and the samples were separated on an agarose gel followed by purification of the samples. FIG. 22 shows an agarose gel with the PCR products of the fluorescent labelled aptamers in lanes 1-7. By comparison with a molecular weight marker in lane M, it can be seen the sizes of the aptamers are around 100 bases, as expected. The PCR control (no template DNA) sample in lane 0 is clear as expected. The PCR products were purified by using spin columns.

### 4.3.1.2 Fluorimetry

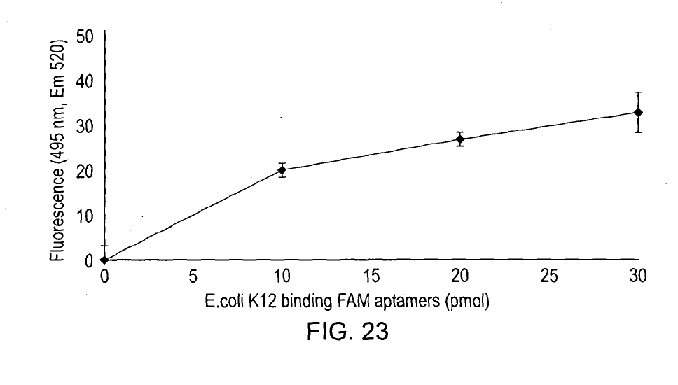

The detection of E. coli K12 binding FAM-labelled aptamer pool 9 was performed by fluorimetry using a fluorescence plate reader. The fluorescence of the E. coli K12 samples incubated with different aptamer concentrations (10 pmol, 20 pmol and 30 pmol) are shown FIG. 23. The greater the number of bound aptamers the higher the fluorescence can be seen in the figure. It can be seen that when the aptamers has been added the fluorescence values are significantly higher (F=71.85, p=3.98×10⁻⁶). The results show that 10 pmol addition of the aptamers is enough to detect the E. coli K12 bacterial cells (F=81.8, p=8.2×10⁴). These results show that the aptamers have bound to live E. coli K12 cells. This method developed for aptamer detection is easy to perform, repeatable and will be used in further aptamer characterisation experiments.

### 4.3.1.3 Fluorescence Microscope

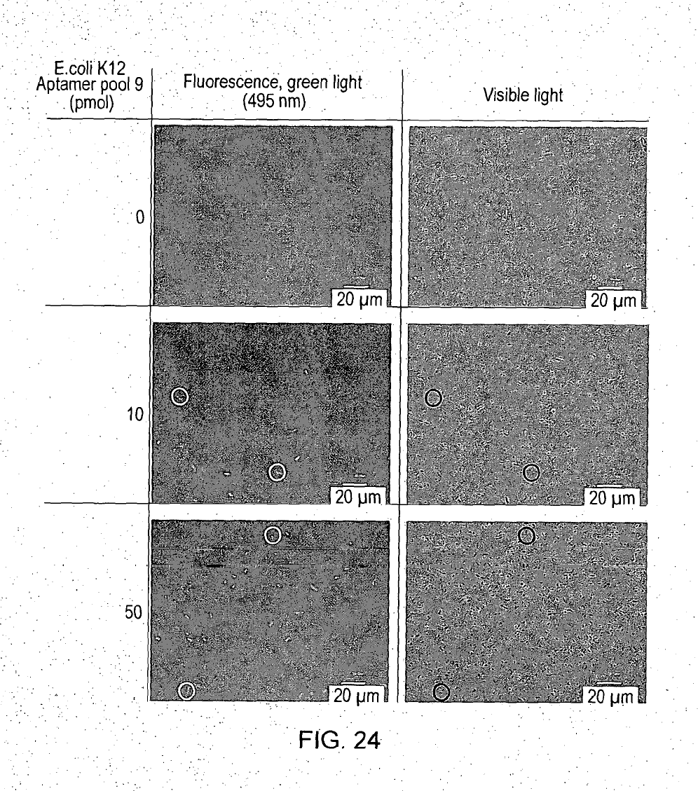

Fluorescence microscopy was used to visualise the bacterial cells with aptamers bound to them. The images were taken from each field with fluorescence and visible light. Images of negative control samples with no added aptamers (0 pmol) and samples with 10 pmol and 50 pmol aptamers are shown in FIG. 24. The results show that when FAM-labelled aptamers are not added (0 pmol) (left hand side), no fluorescent dots can be seen but the dots can be seen when aptamers are added (10 pmol and 50 pmol), as expected. This indicates that the aptamers have bound to the live E. coli K12 cells. By comparing the fluorescence images (left hand side) to the images taken with a visible light (right hand side), one can see that there are more dots on the light microscope images that on the fluorescence images. It could be possible that the used aptamers bind to cells which may be in a specific stage of the cell cycle.

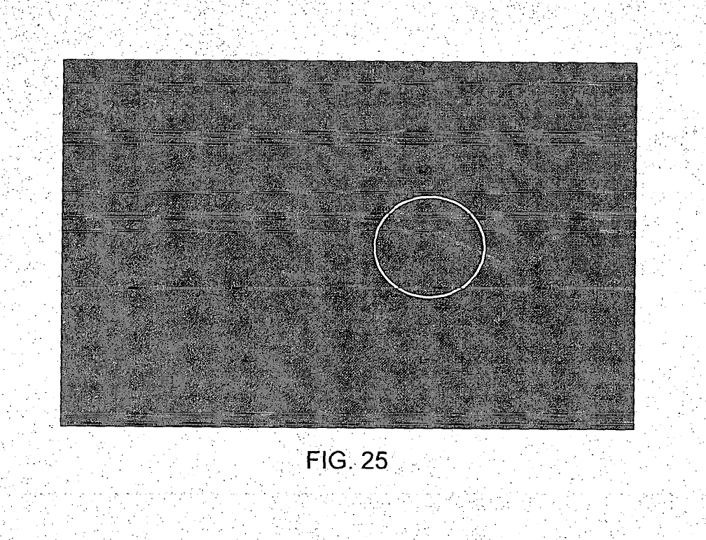

An enlarged fluorescence microscopy image of the bound aptamers is presented in FIG. 25. The long structures can often be seen in the fluorescence images taken in this study. This structure may indicate that the cells, which are detected with the aptamers, are in a dividing stage.

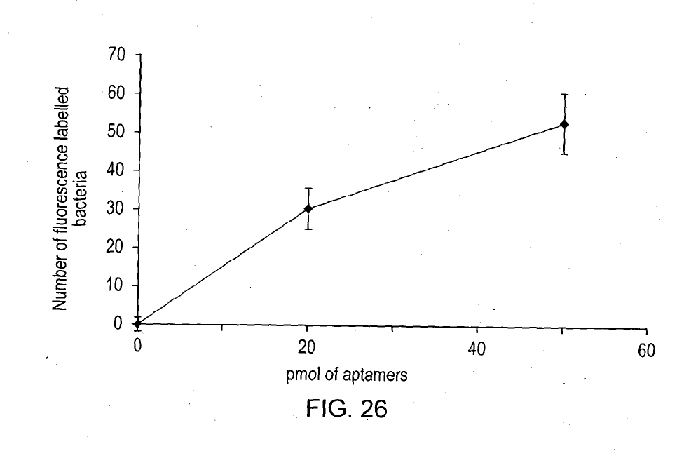

The fluorescence images were taken from six random fields and the bacterial cells with fluorescent labels were counted. The results are shown in FIG. 26. It can be seen that with more aptamers added more fluorescent labelled bacterial cells (dots) are detected. The number of fluorescent labelled bacteria is significantly higher when 50 pmol aptamers have been added compared to 20 pmol aptamer addition (F=34.8, p=1.5×10⁻⁴). On the microscope slide, the bacterial cells are not divided even and therefore the images taken from random fields might have variable results. In some of the images much more bacterial cells can be seen comparing to another image taken from a same microscope slide. The counting of the bacterial cells from the microscope slides is also time-consuming but the method is suitable for visualising the binding of the FAM-labelled aptamers.

### 4.3.2 Optimal Binding Time of the Aptamers

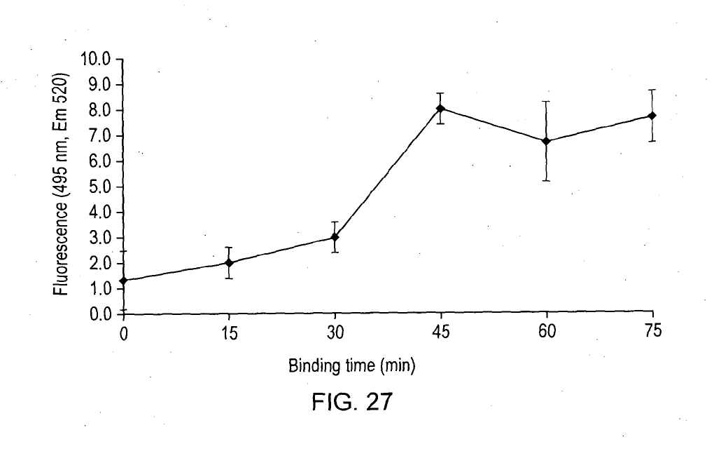

Optimal binding time of the aptamers was measured by incubating the aptamer pool 9 with E. coli K12 and measuring the fluorescence after different time points. The first sample collected was after 0 minutes of incubation and then samples were collected every 15 minutes until 75 minutes. The samples were washed three times before the fluorescence was measured. The fluorescence values are presented in FIG. 27. It can be seen that the highest fluorescence values (best binding of the aptamers) is achieved after 45 minutes incubation. Next sample collected (60 min) is having a lower fluorescence. For the 75 min sample the fluorescence increased again back to the same level with the 45 min sample. The lower fluorescence in 60 min sample might be due to a loss of bacterial cells in one of the replicates during the washes and therefore the measured fluorescence is slightly lower. Also the aptamer concentration may have been lower in this sample than in the other samples. However, there is no significant difference between the samples 45 min, 60 min and 75 min (F=1, p=0.4). It can also be seen that no significantly higher binding can be detected when the samples were incubated for 15 minutes (F=2, p=0.2) but 30 min incubation was enough to see significant increase in the binding (F=12.5, p=0.02). Incubation time 45 minutes was routinely used in following experiments.

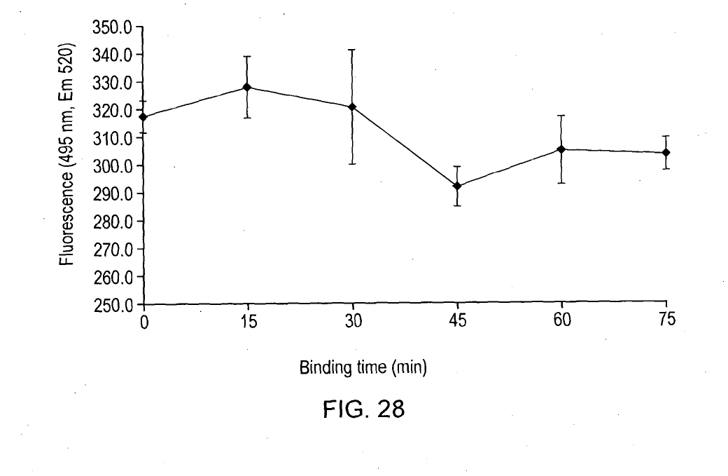

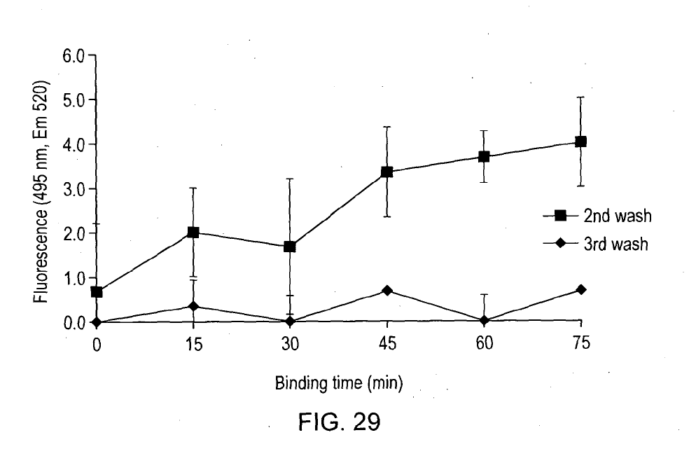

The washes of the optimal binding time samples (0, 15, 30, 45, 60 and 75 min) were collected and the fluorescence was measured to determine the number of washes needed to wash off non-binding aptamers. The fluorescence values for non-binding aptamers after the first wash are presented in FIG. 28, and after the second and third wash in FIG. 29. It is noticeable that the fluorescence values are very high (fluorescence 290-330) after the first wash (FIG. 28) comparing to the second or third washes (fluorescence less than 4) (FIG. 29). This indicates most of the non-binding aptamers are washed off after the first wash and three washes is enough to wash off the non-binding aptamers. In FIG. 29, it can be see that the 60 min sample has a lower fluorescence than the 45 min or 75 min sample. As stated above, it is possible the actual aptamer concentration added to the sample has been lower by mistake. It can also be seen that the fluorescence of the first wash (FIG. 28) is much higher than the fluorescence of the actual samples where the aptamers have bound to the E. coli K12 (FIG. 27). There can be several reasons for such a high number of non-binding aptamers. It can be possible that the most of the denatured DNA aptamers renature back to their double stranded form with their complementary strands. This is instead of forming the structure that allows aptamers to bind to their target when mixed with the bacterial cells. Another reason for the low binding of the aptamers can be that there are not enough bacterial cells serving the binding sites for the aptamers. It has previously been demonstrated that these E. coli K12 binding aptamers do not detect all of the E. coli K12 cells in the solution (FIG. 24). In FIG. 28 it can also be seen that less fluorescence was detected in the 45 min sample than in the other samples. This may indicate that more aptamers have bound to the bacterial cell surface and have not been washed off. In conclusion, 45 minutes was demonstrated to be an optimal binding time for the aptamers and three washes can be used to remove non-binding aptamers from the solution.

### 4.3.3 Binding of the Aptamer Pool 3, 5, 7 and 9

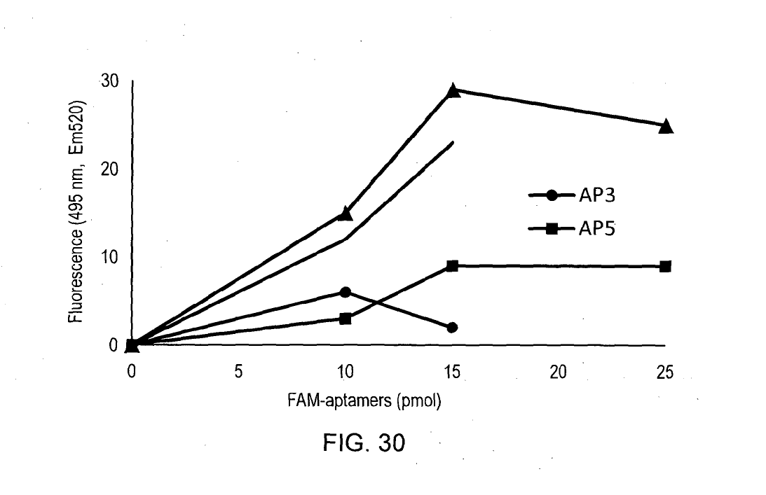

Aptamers were selected to bind to live E. coli K12 bacterial cell by repeating the selection process nine times. The binding of the selected aptamer pool 9 was previously demonstrated. In this study the binding of the previous aptamer pools were tested in order to see if the binding increases during the selection process. FAM-labelled aptamer pools 3, 5, 7 and 9 were incubated with E. coli K12 bacterial cells and the fluorescence of the samples was measured. The fluorescence values of each pool are presented in FIG. 30. Aptamer pool 5 and 9 were tested with three concentrations (10 pmol, 15 pmol and 25 pmol) while aptamer pools 3 and 7 were tested with two different concentrations (10 pmol and 15 pmol). That is because the PCR yield (aptamers) was smaller for the aptamer pool 3 and 7. The PCR yield of different aptamer pools might vary depending on the quality of the template or the amount of the aptamer molecules in each sample. The results show that the highest values were obtained for pools 7 and 9. This indicates that at least seven or nine rounds of selection have to be performed in order to achieve a specific pool of aptamers. If aptamers were further selected, for example aptamer pool 10, it could be possible to maintain even more specific pool. In this study, pool 9 was selected to be enough specific to bind to E. coli K12 bacterial cells.

4.3.4 Specificity of the E. coli K12 Binding Aptamers
4.3.4.1 Binding of the Aptamers to E. coli B, B. subtilis and S. aureus

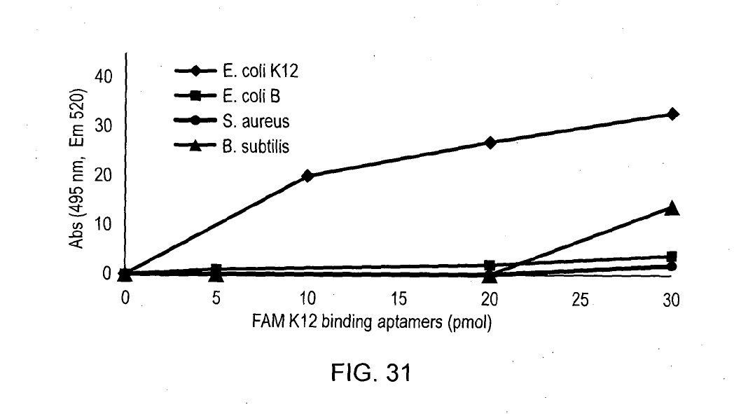

The fluorescent labelled aptamer pool 9 was incubated with E. coli K12, E. coli B, B. subtilis and S. aureus cultures with three different concentrations. For E. coli K12 10 pmol, 20 pmol and 30 pmol aptamers were used and for E. coli B, B. subtilis and S. aureus) 5 pmol, 20 pmol and 30 pmol aptamers were used. The fluorimetry test was performed for all samples. The fluorescence values are presented in FIG. 31. In the figure it can be seen that the more aptamers there are in the sample the higher the fluorescence. The highest fluorescence values can be seen in E. coli K12 sample, even when small amounts (5-10 pmol) of aptamer are added. The fluorescence values for the other samples (E. coli B, S. aureus and B. subtilis) are very low. B. subtilis has given a higher fluorescence value when 30 pmol aptamers have been added. The higher fluorescence might not be actual binding to bacterial cells and can possibly be caused by the clusters the bacteria forms while growing in broth. The aptamer concentration 20 pmol was selected to use in the following specificity experiments because it shows high fluorescence for E. coli K12 but not much fluorescence for E. coli B, S. aureus or B. subtilis.

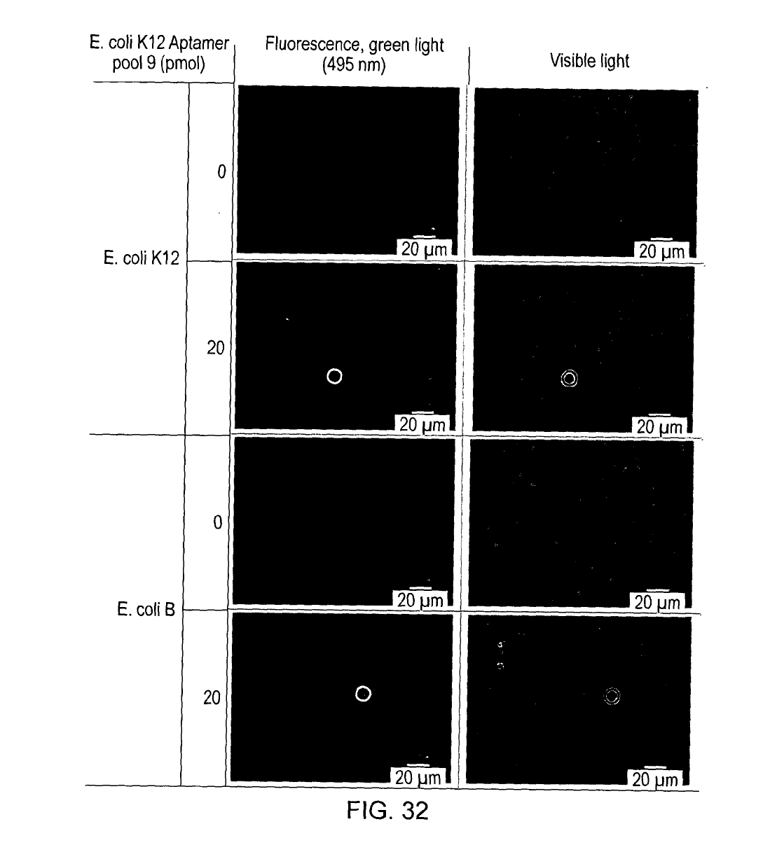

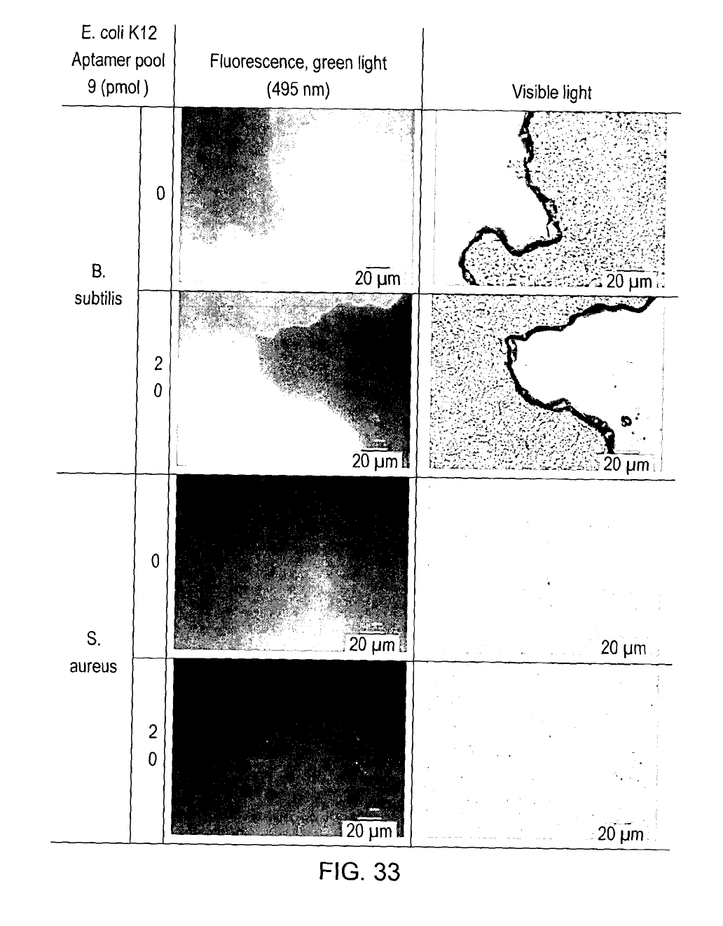

The fluorescence microscope images were taken of each sample (E. coli K12, E. coli B, S. aureus and B. subtilis) from five random fields and the fluorescent dots were counted. The images were taken with a green fluorescence light and with a visible light. The microscopy images of 20 pmol samples of E. coli K12 (positive control) and E. coli B are presented in FIG. 32 and of B. subtilis and S. aureus are presented in FIG. 33. It can be seen in the microscope images, when the FAM-labelled aptamers are added, that bacterial cells with fluorescent labels are visualised as green dots. When the FAM aptamers were incubated with E. coli B, four fluorescent dots can be detected in the image. This indicates that the aptamers might also bind to E. coli B, but not as strongly as to E. coli K12. It can also be possible that the dots are not actual binding to bacterial cell surface but are the remaining fluorescence aptamers in the solution instead. In FIG. 33 no separate bacterial cells can be seen on B. subtilis samples as the bacterial cells were growing in clusters in the broth. The clusters did not break down to separate bacterial cells when the samples were mixed and that can also be seen on the microscope images. The negative control sample (0 pmol) for B. subtilis looks the same as the sample where the aptamers have been added (20 pmol). Due to the difficulties of handling B. subtilis, it was not used in following experiments. In FIG. 33 S. aureus samples are shown. It can be seen in the samples, where FAM-labelled aptamers were added (20 pmol), that some of the bacterial cells are detected with the aptamers but it can also be seen that the number of detected bacteria is much smaller than when the aptamers are added to the positive control samples (E. coli K12, FIG. 32). This result indicates that some of the aptamers might bind to S. aureus, but the binding is not as strong as to E. coli K12. When comparing the S. aureus (FIG. 33) images taken with a visible light (right hand side) to the image taken with fluorescence light (left hand side), it can be seen that there is an area where more bacterial cells are in the same place. This area can also be seen brighter green in the fluorescence image. It might be possible the fluorescence in the images is not actual aptamer binding to bacterial cells but is caused by the FAM-labelled aptamers remained in the suspension.

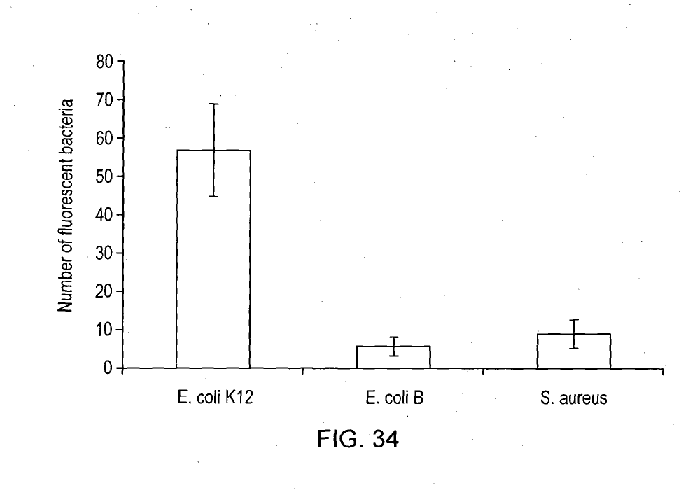

Fluorescent labelled bacterial cells were counted from five images (n=5) of E. coli K12, E. coli B and S. aureus. The results are presented in FIG. 34. It can be seen that the E. coli K12 samples have significantly more bacterial cells with aptamers bound to them than the E. coli B and S. aureus samples (F=75.8, p=1.55×10⁻⁷). The aptamers are specifically binding to E. coli K12 but very low detection levels can be seen with E. coli B and S. aureus.

4.3.4.2 Detection of E. coli K12 from a Bacterial Mixture with FAM-Labelled Aptamers

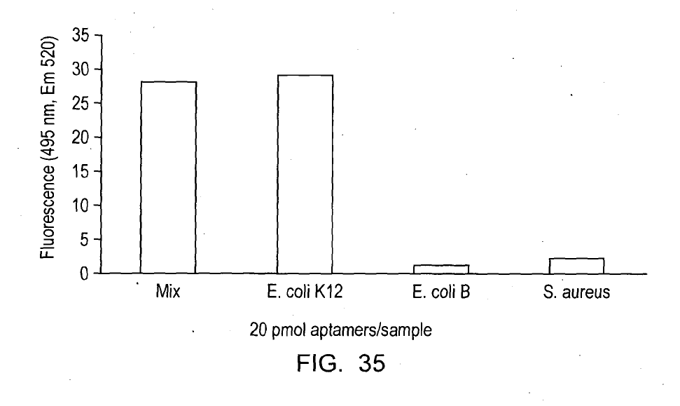

The capability of the aptamer pool to detect E. coli K12 from a mixture of the bacterial cells was tested. FAM-labelled aptamers were incubated with a mixture of bacterial suspension (E. coli K12, E. coli B and S. aureus) and each suspension individually. In FIG. 35, the fluorescence values of the samples are presented. In the diagram, it can be seen the highest fluorescence was detected from the mixture of these three bacterial suspensions (Mix) as expected. Minor fluorescence can be detected from E. coli B and S. aureus samples. The positive control sample (E. coli K12) has the strongest fluorescence, as expected. There is no big difference between the fluorescence values of the mixture sample and the positive control sample. These results indicate the aptamers can detect E. coli K12 from a mixture of bacterial cells.

4.3.4.3 Binding of the Aptamers to L. acidophilus

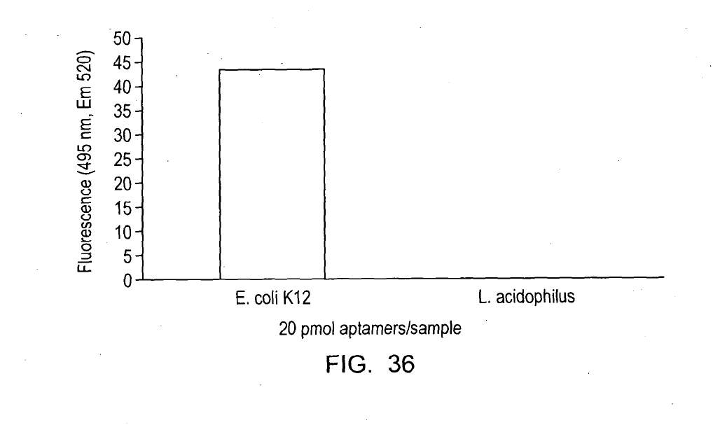

The specificity test was performed with L. acidophilus and E. coli K12 was used as positive control. Aptamer pool 9 (20 pmol) was incubated with bacterial cells and the fluorescence of the samples was read by the plate reader. The results are presented in FIG. 36. In the diagram, it can be seen that no fluorescence was detected in gram-positive L. acidophilus samples. The fluorescence of the positive control sample (E. coli K12) is significantly higher than L. acidophilus. This result confirms the aptamer pool 9 is specifically binding to E. coli K12 and no binding to L. acidophilus can be detected.

### 4.3.5 Fluorescence Microspheres

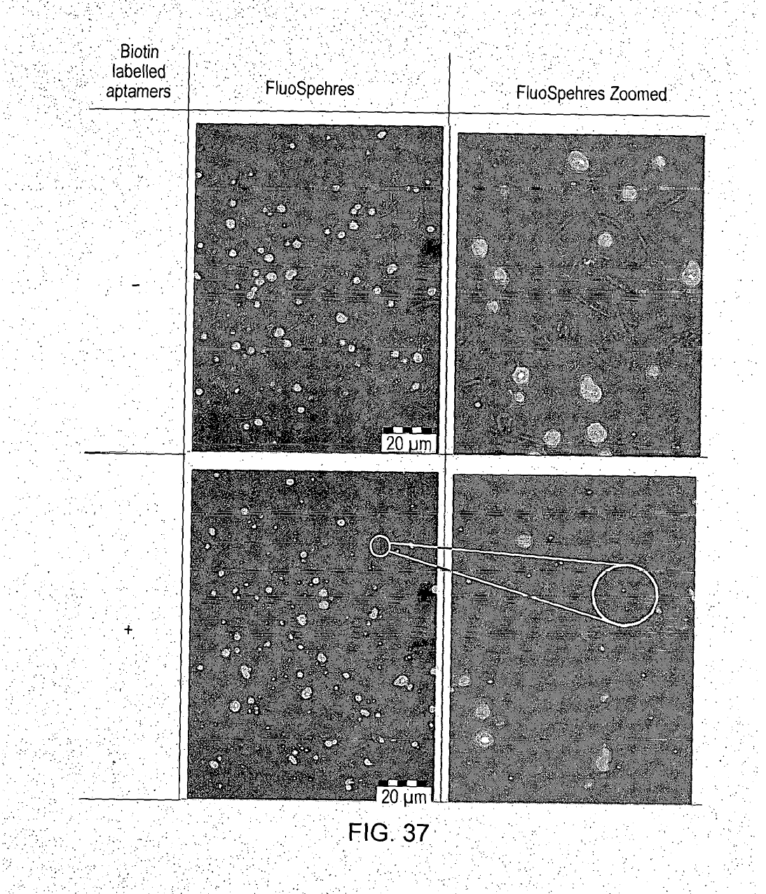

Fluorescent labelled aptamers binding to smaller bacterial cells than E. coli K12 might be difficult to detect under the microscope. Fluorescence microspheres technique was developed to detect the aptamer binding to small bacterial cells. Biotin labelled aptamer pool 9 was produced and the aptamers were incubated with E. coli K12 bacterial cells. The FluoSpheres were added and samples were incubated in order to let the microspheres bind to biotin label of the aptamers. The samples were visualised under the fluorescence microscope and the images were taken. Examples of the microscope images are shown in FIG. 37. The bacterial cells can be seen in the images as well as the microspheres because the images were taken with a green fluorescence light and a visible light. Normal sized images are on left hand side and on right hand side are the zoomed images where the binding of the FluoSpheres can be seen. When comparing the negative control sample (−) to the sample (+) where the biotin aptamers have been added, it can be seen that some of the microspheres have bound to the bacterial cells surface. This kind of binding cannot be seen in the control sample. The results indicate that the biotin-labelled aptamers and the fluorescence microspheres can be used in detection of aptamer binding. This method can possibly be used in detection of the aptamer binding to bacterial cells smaller than E. coli K12.

4.3.6 Aptamer Detection of Live and Dead E. coli K12 Bacteria Cells

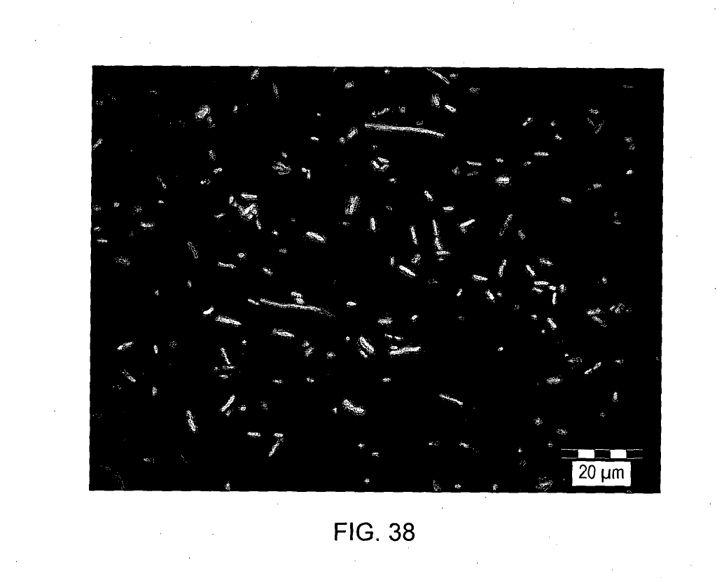

The Live/Dead BacLight staining was performed in order to see if live or dead E. coli K12 bacterial cells are detected with the aptamers. The FAM-labelled aptamers were first incubated with E. coli K12 and the Live/Dead staining was performed. The FAM-labels were difficult to see because of the brighter colour from Live/Dead staining. In FIG. 38 a fluorescence microscope image is shown. It can be seen that it is impossible to detect if the aptamers have bound to green, live bacterial cells. On red dead cells some green colour can be detected but this colour might also be derived from the Live/Dead staining kit where the colour has partly stained the bacterial cells.

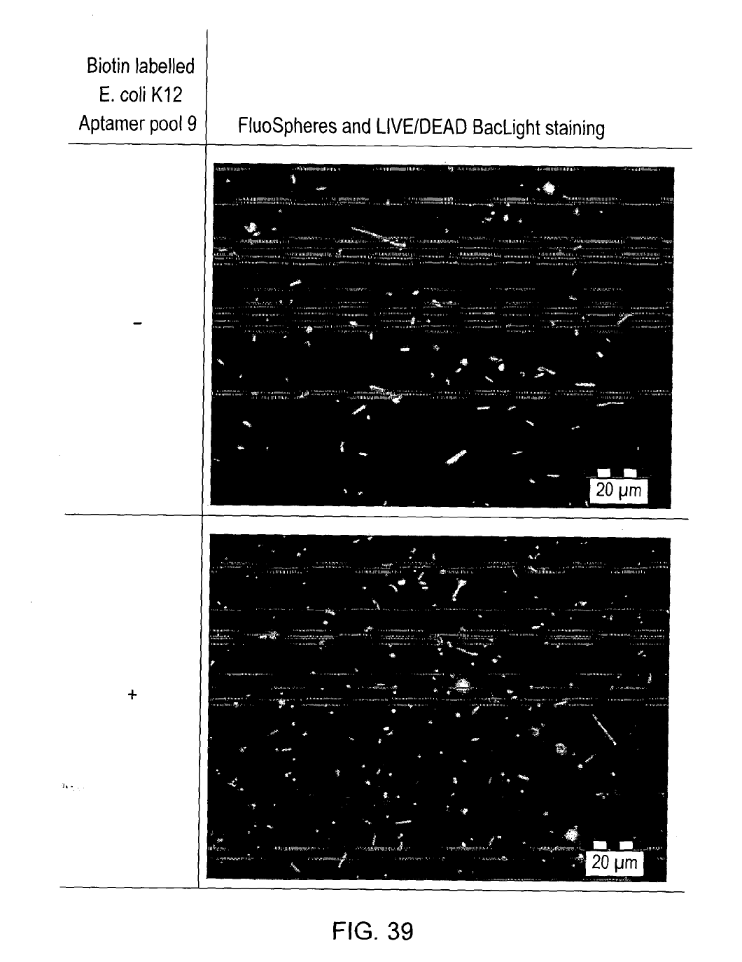

The biotin-labelled aptamer pool 9 was then incubated with E. coli K12 bacterial cells and the FluoSpheres were attached to them followed by Live/Dead staining of the cells. The microscope images are presented in FIG. 39. It can be seen that some of the fluoSpheres have bound to bacterial cell surface on negative control samples (−) where biotin labelled aptamers have not been added. This indicates that the fluoSpheres might bind to bacterial cell surface. Not much binding can be detected on the sample (+) where the biotin labelled aptamers have bound to bacterial cells surface. Some of the molecules could have been washed off during the staining. Also, the bond strength between the aptamer and the bacterial cell in not known and it is possible that this bond breaks when the non-binding FluoSpheres are washed off. The Live/Dead staining can also affect to the binding.

### 4.4 Conclusions

Aptamers were selected to bind specifically to E. coli K12 live bacterial cells. The binding was first detected with a method based on an enzymatic reaction. This method was time consuming and the results were unreliable. The easier and more reliable fluorescence based method was developed and by using this method the binding properties of the aptamers were analysed.

The binding of the fluorescent-labelled aptamers was tested by comparing the fluorescence values measured. This method developed makes the testing of the aptamer binding easier and allows to comparing different samples to each other. The fluorescent labelled aptamer binding can also be visualised under a fluorescence microscope. However, this method cannot be used for statistical analysis, is time consuming and is not very reliable as the bacterial cells are not spread consistently over a microscope slide, but it gives interesting visual information about the aptamer binding.

It was found that the optimal binding time for the aptamers is 45 minutes and the binding is not significantly improving when the incubation time was increased. Three washes are enough to wash off the non-binding aptamers. The specificity tests indicated that the aptamers are binding specifically to E. coli K12 bacterial cells and that cells can also be detected from a mixture of bacterial cells. Not all bacterial cells are easy to see under a microscope as they might be smaller in size and move faster than E. coli K12. An alternative method was introduced where the same principles of visualising the samples under the fluorescence microscope were used. In this experiment the aptamers were labelled with a biotin-label and fluorescent labelled microspheres were attached to them. The samples were then visualised under the microscope. The method tested in this study shows the aptamer binding can be detected with the fluorescence beads.

The results show detection of the aptamers from complex matrix.

### Example 5

### Identification of the Escherichia coli K12 Binding Aptamers Sequences

### 5.1 Introduction

The aptamers have been selected to bind live E. coli K12 bacterial cells. The binding has been demonstrated by an enzymatic method (3.3.4) and by fluorescence based methods (4.3.1). The selected aptamers are specifically binding to E. coli K12.

In this study the aptamer pool 9, which is specific to E. coli K12, was cloned with a commercial cloning kit and competent bacterial cells. The clones were analysed and the cloned plasmids were extracted and sequenced. From the plasmid vector sequence the aptamer sequence was identified and analysed. Since the synthesis of long (100b) nucleotide sequences is being difficult and expensive, the nucleotide sequences of the aptamers are often reduced. James (2007) stated that the reduction below 60 nt is almost essential for efficient chemical synthesis and a size of 40 nt or below is desirable on grounds of cost of goods. From the aptamer's secondary structure the shortened aptamer sequences were isolated and synthesised with a fluorescent label. The fluorescence based detection methods, fluorimetry and fluorescence microscopy, were used to detect the aptamer binding to the bacterial cells. The specificity of the aptamers was tested by incubating the fluorescent aptamers with E. coli B and S. aureus followed by fluorescence measurements.

Once the aptamer sequences are identified the aptamers can be chemically synthesised.

### 5.2 Methods

### 5.2.1. Cloning of Aptamers

The binding of aptamer pool 9 to E. coli K12 has been demonstrated (3.3.4; 4.3.1). So far, the aptamer pools with unidentified sequences have been produced by PCR. In order to synthesise the aptamers the individual sequences need to be determined. This was done through cloning of the aptamers.

### 5.2.1.1 Ligation and Transformation

The E. coli K12 aptamer pool 9 was produced by PCR (3.3.2) and ligated into a pGEM-T easy vector using a method described in 2.3.12.1. It has been suggested to use 1:1 insert:vector ratio for the control insert DNA for pGEM-T easy vector while a optimisation of the ligation reaction for aptamers was recommended (Promega Technical Manual). The formula for molar ratio calculation was:

ng of vector×kb size of insert×insert:vector molar ratio=ng of insert kb size of vector

In this study the insert:vector ratio was optimised for ratios 1:4, 1:2 and 1:1. The PCR product (insert) concentration was estimated to 0.8 ng/μl and the PCR product size was 100 b (0.1 kb). The pGEM-T easy vector (50 ng) size is 3 kb. Therefore, the PCR product amounts for this reaction were 0.4 ng (1:4), 0.8 ng (1:2) and 1.7 ng (1:1). The ligation reaction components are listed in the Table below.

TABLE
Ligation reaction components

| Reaction | Insert:vector molar ratios | | | | Positive |
|---|---|---|---|---|---|
| component | 1:4 | 1:2 | 1:1 | Background | control |
| Ligation buffer | 5 μl | 5 μl | 5 μl | 5 μl | 5 μl |
| Vector pGEM-T | 1 μl | 1 μl | 1 μl | 1 μl | 1 μl |
| Easy | | | | | |
| PCR product | 0.5 μl | 1 μl | 2 μl | — | control |
| | | | | | insert 2 μl |
| Ligase | 1 μl | 1 μl | 1 μl | 1 μl | 1 μl |
| H₂O | 2.5 μl | 2 μl | 1 μl | 3 μl | 1 μl |

The transformation of the ligation reaction (2 μl) into the competent cells was done as described in section 2.3.12.1. After the transformation the samples were incubated in SOC medium before plating 100 μl of suspension on LB plates with ampicillin/IPTG/X-gal. The colonies were counted from overnight grown selective plates (2.3.12.2). From the selective plates, eight positive white colonies were randomly selected and streaked on a new indicator plate. The plates were incubated overnight.

### 5.2.1.2 PCR Analysis of the Positive Colonies

The colonies on the selective plates were analysed to see if the ligation and transformation had worked. The PCR analysis was performed for eight samples (CI1-CI8) from the overnight selective plates with PCR Supermix HiFi (2.3.12.3) and the aptamer primers PR1 and PR2. The templates used in this reaction were taken directly from the colonies. The initial denaturation of PCR cycle breaks down the bacterial cell walls releasing the plasmid DNA with an insert. The primers used are the same as the primers used for aptamer production. If the cloning of the insert (aptamer) and the transformation of the vector inside the bacterial cells have been successful, a 100 bp long band should be visible on the agarose gel images. The PCR control (no template DNA) and a plasmid vector control (template 0.5 μl vector) samples were performed. The plasmid DNA control shows if the plasmid itself has binding sites for the primers used in this reaction. The PCR products were separated on an agarose gel.

### 5.2.1.3 EcoRI Restriction Analysis for Plasmid Vectors

The cloned eight colonies were also tested with a restriction analysis to see if the cloning has worked. Restriction analysis (2.3.12.5) was performed to confirm the results of PCR analysis. The colonies were inoculated into LB-media and the plasmid was purified from the overnight grown culture (2.3.12.4). EcoRI enzyme has digestion sites in the both sides of the insert and therefore was selected to digest the insert out from the plasmids (CI1-CI8). The digestion sites can be seen in the sequence of the pGEM-T Easy vector in FIG. 11. The samples were separated on an agarose gel (2.3.2).

5.2.2 Binding of the Cloned Aptamers to E. coli K12

Two cloned colonies (CI1 and CI2) were randomly selected to test if they are binding to E. coli K12. The clones were produced by PCR (2.3.1) under the same conditions using the fluorescent FAM-primers as in the usual aptamer production. The template used in this reaction was a nested template for the cloned aptamers in dilution 1:60. The PCR products were separated on agarose gel followed by purification of the PCR product.

The overnight grown E. coli K12 culture was prepared as previously described (2.3.9.1). The bacterial suspension (100 μl) was incubated with 10 pmol, 20 pmol and 40 pmol of aptamers. The samples were washed and the fluorescence was measured by the plate reader (2.3.9.3).

### 5.2.3 Sequencing of Cloned Vectors and Aptamer Analysis

In order determine out the aptamer sequences, the cloned aptamers were sequenced directly from the plasmid vectors. According to the results of the PCR analysis and the restriction analysis four positive samples were selected for the sequencing. Plasmid DNA samples CI1-CI4 (30 μl each) were sequenced (2.3.12.6). The aptamer sequences were identified from the vector sequence by identifying the primers binding sites of the primers PR1 and PR2 from the vector sequence. Between the primer sequences the aptamer sequence with 40 nucleotides long random sequence were found.

The aptamer binding to its target is dependent on its secondary structure. The possible secondary structures of the aptamer sequences were analysed as previously described (2.3.12.7). From the cloned and analysed sequences four aptamers (1AptK12, 2AptK12, 4AptK12 and 6AptK12) were selected and synthesised with a fluorescent FAM-label in its 5′ end. It was decided to use four different sequences for the analyses and these four aptamers were selected because of their matching structure to the original 100 nucleotides secondary structure. Also the energy needed to break down the structure (ΔG) was taken into account when selecting the aptamers to be synthesised and to be used for further analysis. The smaller the ΔG is the stronger the structure.

5.2.4 E. coli K12 Binding Aptamers
5.2.4.1 Binding of the Aptamers to E. coli K12

The binding properties of the sequenced aptamers were tested. Aptamers 1AptK12, 2AptK12, 4AptK12 and 6AptK12 were synthesised with 5′ FAM-label and the binding to E. coli K12 was tested. The overnight grown E. coli K12 culture (2.3.9.1) was washed and incubated with the aptamers (10 pmol, 20 pmol, 50 pmol and 100 pmol) in triplicate. Due the synthesis of single stranded molecules the denaturation of the aptamers is not necessary. The aptamers (10 μl) were added to 100 μl bacterial suspension in BB. The fluorescence of the samples was read by the plate reader (2.3.9.3). The values were tested with the analysis of variance (ANOVA). To visualise the binding of the aptamers, 50 pmol samples were selected and visualised under the fluorescence microscope (2.3.9.2).

### 5.2.4.2 Specificity of the Aptamers

The binding of the aptamers 1AptK12, 2AptK12, 4AptK12 and 6AptK12 to E. coli B and S. aureus was tested. E. coli K12 was used as a positive control. A mixture of the aptamers (50 pmol) was used in the binding reaction (2.3.9.1) in triplicate. The samples were incubated and after the washes the fluorescence was measured by the plate reader (2.3.9.3) and the samples were visualised under the fluorescence microscope (2.3.9.2). The fluorescence values were analysed using the statistic test ANOVA. The same protocol was then repeated with individual aptamers (20 pmol) in order to see the differences between different aptamers. Due the problems with the plate reader the sensitivity had to be changed from 50 to 75 in order to read the plate.

### 5.3 Results and Discussion

### 5.3.1 Cloning of Aptamers

### 5.3.1.1 Ligation and Transformation

The aptamer pool with unknown aptamer sequences was cloned to determine the specific aptamer sequences. Aptamer pool 9 was amplified by PCR and cloned with pGEM-T Easy vector and JM109 competent cells. The colonies on indicator plates were counted and a number of positive (white) and negative (blue) colonies is presented in the Table below. The number of colonies on the plates was small. The positive control sample was spread on a selective plate, where the ampicillin used was not fresh and therefore might have lost its activity. On a normal selective plate the positive control sample express the colonies if the transformation has been successful. Due the inactive ampicillin the plates were full of colonies. When the ampicillin is normally used, the positive colonies are only growing on the plates due the ampicillin resistance of the bacteria. Even though the positive control sample was full of colonies, further analyses of the actual samples were performed. Eight white colonies were selected from the plate and streaked on new selective plates. After an overnight incubation on selective plates, five colonies (CI3, CI4, CI6, CI7 and CI8) out of eight expressed the blue colour while three remained white (CI1, CI2 and CI5). The blue colour may be expressed even if the insert has not been ligated into the vector. In a normal situation where the insert has successfully been ligated the reading frame for the lacZ gene has been interrupted. This means that the enzyme β-galactosidase is not expressed and therefore the blue coloured colonies are not formed on the selective plates containing the X-Gal and IPTG. If positive blue colonies are formed the PCR products have been cloned in-frame with the lacZ gene or it may be caused by the mutations (deletions or point mutations) (Promega Technical manual).

TABLE
Number of colonies on indicator plates.

| Insert:Vector | Blue | White | |
|---|---|---|---|
| molar ratio | (negative) | (positive) | Total |
| 1:4 | 3 | 2 | 5 |
| 1:2 | 20 | 21 | 41 |
| 1:1 | 1 | 2 | 3 |
| Background | 1 | 2 | 3 |
| Positive control | ∞ | ∞ | ∞ |

### 5.3.1.2 PCR Analysis of the Positive Colonies

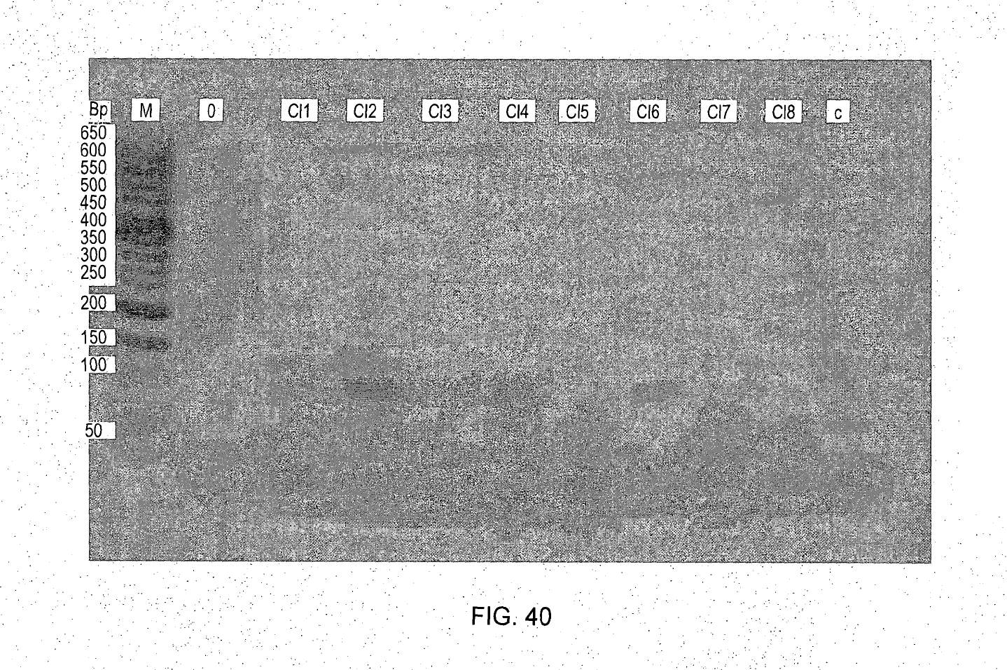

The colonies from the selective plates were analysed to see if the insertion of the aptamers into the vector has been successful. The PCR analysis was performed for the cloned colonies. The aptamer primers PR1 and PR2 were used in this experiment. Therefore, only the colonies with the aptamer sequence and the primer binding sites are amplified in PCR resulting in a 100 bp products. If 100 bp long bands can be seen on a gel the aptamer cloning has been successful. The agarose gels of the PCR amplification products are presented in FIG. 40. It can be seen that the samples CI1, CI2, CI3 and CI4 have a 100 bp band and a very faint band can be seen in sample CI6. This result shows that the cloning of the aptamers has been successful and the cloned insert has been amplified. No PCR product can be seen in sample CI5, CI7 and CI8. The PCR control sample (0) is clear and no amplification products can be seen, as expected. The faint band that can be seen on lane 0 is the primer dimer. No PCR products can be seen in the control sample (c), as expected. This result indicates the insert vector itself does not offer binding sites for the primers used in this experiment. Even the colonies on the second indicator plates were expressing blue colour (CI3, CI4, CI6, CI7, CI8), the PCR analysis gave a positive result for these samples. Also white colonies (CI1, CI2, CI5) were stated to be positive, but according to the PCR analysis, samples CI1 and CI2 were positive and CI5 negative. The results presented here suggest the colour selection is not appropriate for the positive colony selection because also the blue colonies might have the insert in their vector plasmid. In a typical situation the insert inactivates the α-peptide that codes an enzyme, β-galactosidase, resulting in the white colonies on indicator plates. In this study, the β-galactosidase production was not inhibited and therefore positive blue colonies were formed.

### 5.3.1.3 EcoRI Restriction Analysis for Plasmid Vectors

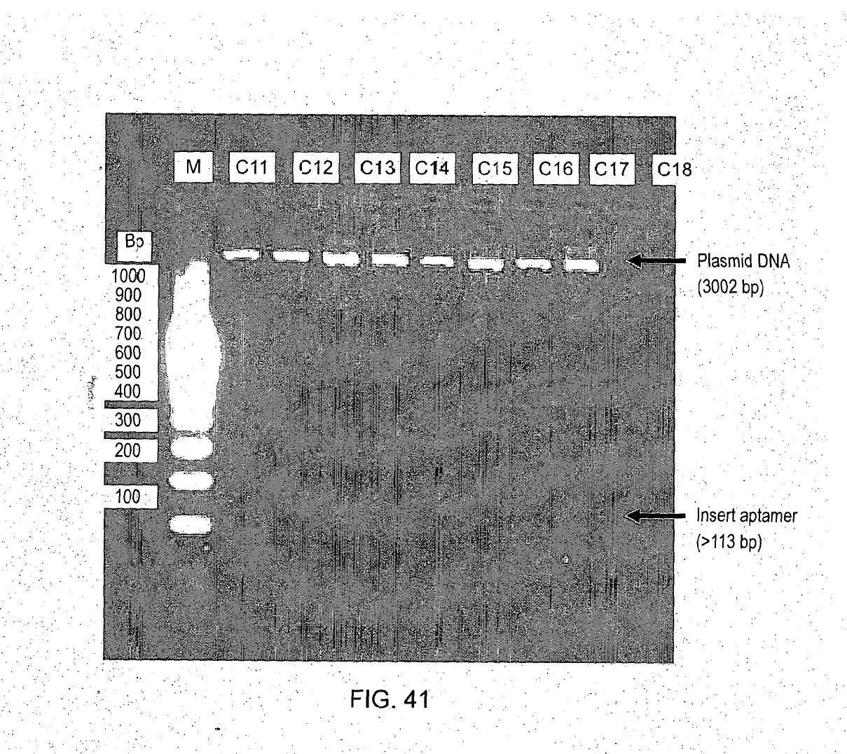

Cloned colonies were analysed with the PRC analysis to see if the cloning has been successful. The same samples were then analysed with a restriction analysis to get a confirmation for the results from the PCR analysis. The plasmids were extracted from overnight grown bacterial cell suspensions. The size of the whole pGEM-T Easy vector is 3015 bp and the insert aptamers are 100 bp. The total size of the cloned plasmid is 3115 bp. The EcoRI cut the insert out from the vector close to the insert adding 13 bases more to the actual insert. Therefore the expected insert size to be seen on a gel is 113 bp and for the vector 3002 bp. The agarose gel for the separated samples is presented in FIG. 41. The pGEM-T Easy vector bands (Plasmid DNA) and the digested inserts (aptamers) are in lanes CI1-CI8. It can be seen that the plasmid purification has been successful in all of the samples. The large plasmid DNA has not moved far on the gel and the range of the molecular weight marker does not reach to it. Also the intensity of the plasmid DNA bands is high when compared to the intensity of the insert bands. The lighting of the image has been changed in order to get the faint insert band visible. The insert band can be seen in all of the samples except CI1 and the band for CI2 is very faint and can hardly be detected. It was expected that a 113 bp band would be seen in sample CI1 when comparing the results for the PCR analysis (FIG. 41) where CI1 was positive. It can be possible that the restriction analysis did not work for this sample, or the DNA concentration is too low to be detected from the gel.

5.3.2 Binding of the Cloned Aptamers to E. coli K12

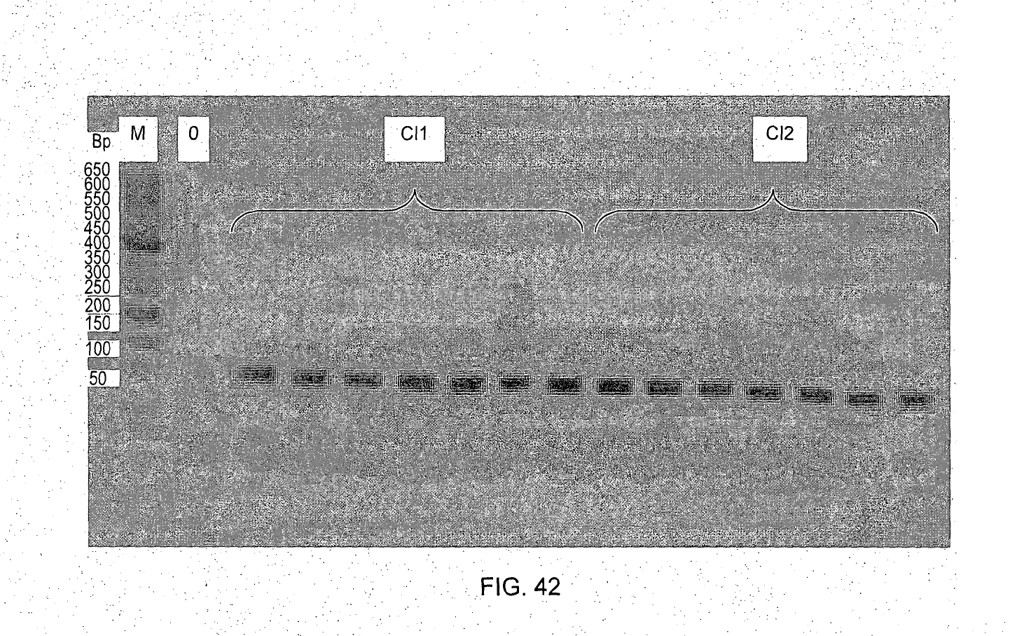

The binding of two aptamer clones with still unknown nucleotide sequences to E. coli K12 was demonstrated to see if these aptamers are still biding their target. The aptamers were produced using the usual aptamer production method. Two different cloned aptamers were used as a template. The agarose gel of the PCR products is presented in FIG. 42. CI1 and CI2 cloned aptamers were produced with FAM-labels. The PCR products are on lanes CI1 and CI2. All samples have a strong 100 bp band. In lane 0 is the PCR control sample where only primers were amplified without a template. No amplification products can be seen in this lane as expected. The faint band (<50 bp) in this lane is the primer dimer. The PCR products were purified with spin columns.

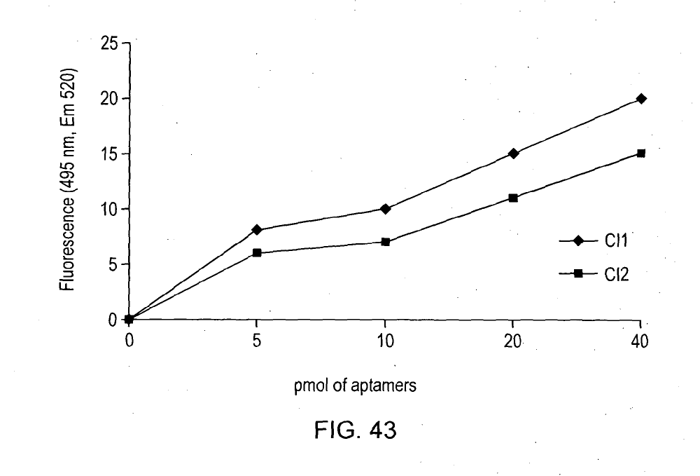

The purified FAM-labelled aptamers were incubated with E. coli K12 using the aptamers in three different concentrations (10 pmol, 20 pmol and 40 pmol). The fluorescence of the samples was measured using a plate reader and the fluorescence values are shown in FIG. 43. It can be seen in FIG. 43 that the fluorescence is higher when more aptamers have been added. This shows that the cloned aptamers can bind to E. coli K12 bacterial cells. The higher fluorescence levels can be detected from sample CI1 than the sample CI2. It is possible that this aptamer binds more strongly to the target than sample CI2. It is possible that these two different samples would have produced curves closer to each other if the replicates had been performed.

### 5.3.3 Sequencing of Cloned Vectors and Aptamer Analysis

The aptamer pool 9 specific to E. coli K12 has been demonstrated to be binding to its target. The aptamers in this pool are still unknown and to obtain the aptamer sequences the cloned aptamers need to be sequenced. Four positive colonies were selected and the plasmid vectors were extracted. The aptamer sequences were determined by sequencing these vectors. By comparing the sequences to the aptamer primer sequences, PR1 and PR2, the aptamer sequences were identified from the vector. Nucleotide sequences of the aptamers are presented in the Table below. The length of the original DNA library, where the aptamers have been selected from, was 100 nucleotide (27 nt and 33 nt long primer binding sites and 40 nt random sequence). In the Table below the 40 nt random sequence is underlined and the primer binding sites are on both sides of the sequences. The T7 and Sp6r sequences of each sample are complement strands to each other as they are sequenced from the same vector in different directions. Some of the aptamer sequences start with the same sequence as the primer PR1 and some of the sequences start with a sequence of primer PR2. This is due the unknown direction when the insert is ligated into the vector. The first sample was 99 nt long (1CI-AptK12). One nucleotide might have disappeared during the sequencing or during the earlier stages of the selection. The rest of the samples were 100 nt sequences (2CI-AptK12, 3CI-AptK12 and 4CIAptK12), as expected. The 99 nt long 1CI-AptK12 can be seen on the agarose gel (FIG. 40) of the PCR analysis. One nucleotide differences cannot be detected on an agarose gel. Although in the gel image, it looks like the band (CI1) is slightly bigger than the band next to it on lane (CI2). On the gel image of the restriction analysis samples in FIG. 41 no bands can be seen in CI1 sample. These results may indicate that this CI1 is different from the others.

Table
Cloned E. coli K12 binding aptamer sequences

| Sample | Size/nt | Primer | Sequence |
|---|---|---|---|
| 1Cl- | 99 | T7 | ACCCCTGCAGGATCCTTTGCTGGTACC |
| AptK12 | | | [symbol] |
| | | | [symbol] AGTATCGCTAATCA |
| | | | GTCTAGAGGGCCCCAGAAT |
| | 99 | Sp6r | ATTCTGGGGCCCTCTAGACTGATTAGCGA |
| | | | TACT [symbol] |
| | | | [symbol] GGTACCAGCAAA |
| | | | GGATCCTGCAGGGGT |
| 2Cl- | 100 | T7 | ACCCCTGCAGGATCCTTTGCTGGTACC |
| AptK12 | | | [symbol] |
| | | | [symbol] AGTATCGCTAATCAGTCT |
| | | | AGAGGGCCCCAGAAT |
| | 100 | Sp6r | ATTCTGGGGCCCTCTAGACTGATTAGCGA |
| | | | TACT [symbol] |
| | | | [symbol] GGTACCAGC |
| | | | AAAGGATCCTGCAGGGGT |
| 3Cl- | 100 | T7 | ATTCTGGGGCCCTCTAGACTGATTAGCGA |
| AptK12 | | | TACT [symbol] |
| | | | [symbol] GGTACCAGCAA |
| | | | AGGATCCTGCAGGGGT |
| | 100 | Sp6r | ACCCCTGCAGGATCCTTTGCTGGTACC |
| | | | [symbol] |
| | | | [symbol] AGTATCGCTAATC |
| | | | AGTCTAGAGGGCCCCAGAAT |
| 4Cl- | 100 | T7 | ACCCCTGCAGGATCCTTTGCTGGTACC |
| AptK12 | | | [symbol] |
| | | | [symbol] AGTATCGCTAATCA |
| | | | GTCTAGAGGGCCCCAGAAT |
| | 100 | Sp6r | ATTCTGGGGCCCTCTAGACTGATTAGCGA |
| | | | TACT [symbol] |
| | | | [symbol] GGTACCAGCAAA |
| | | | GGATCCTGCAGGGGT |

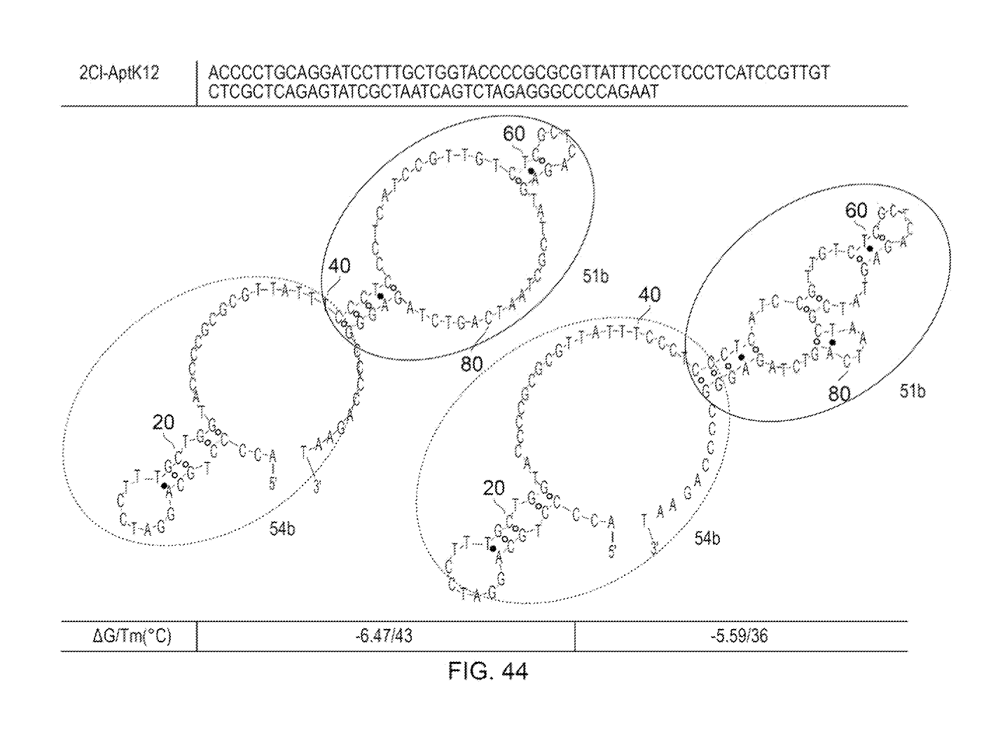

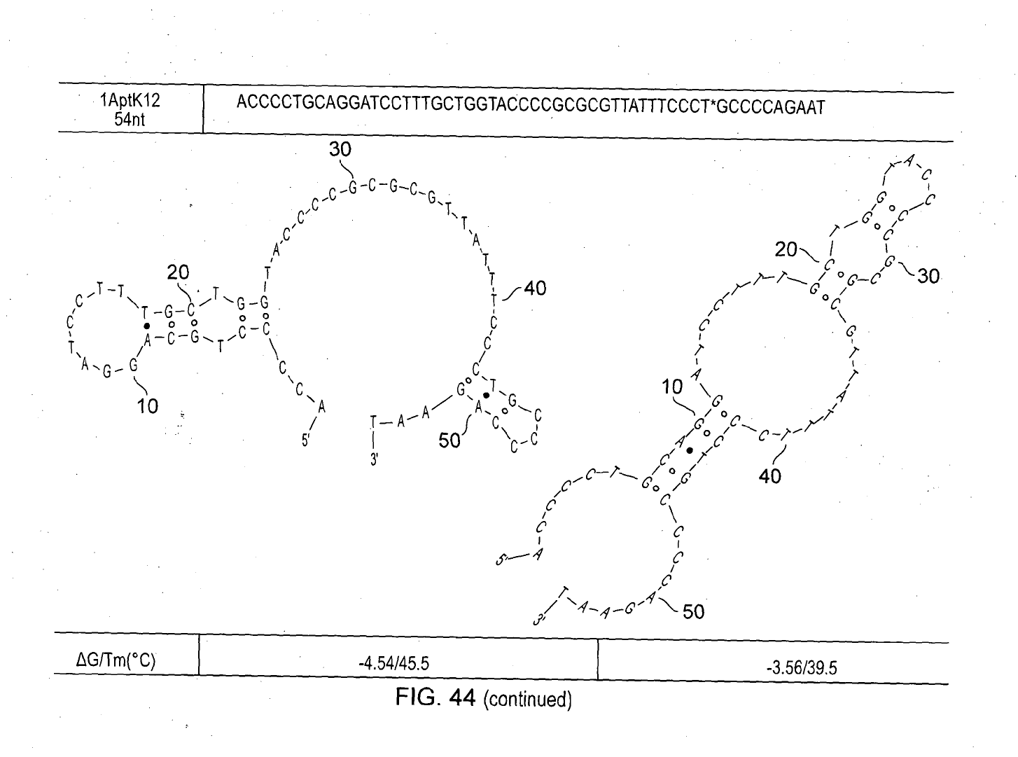

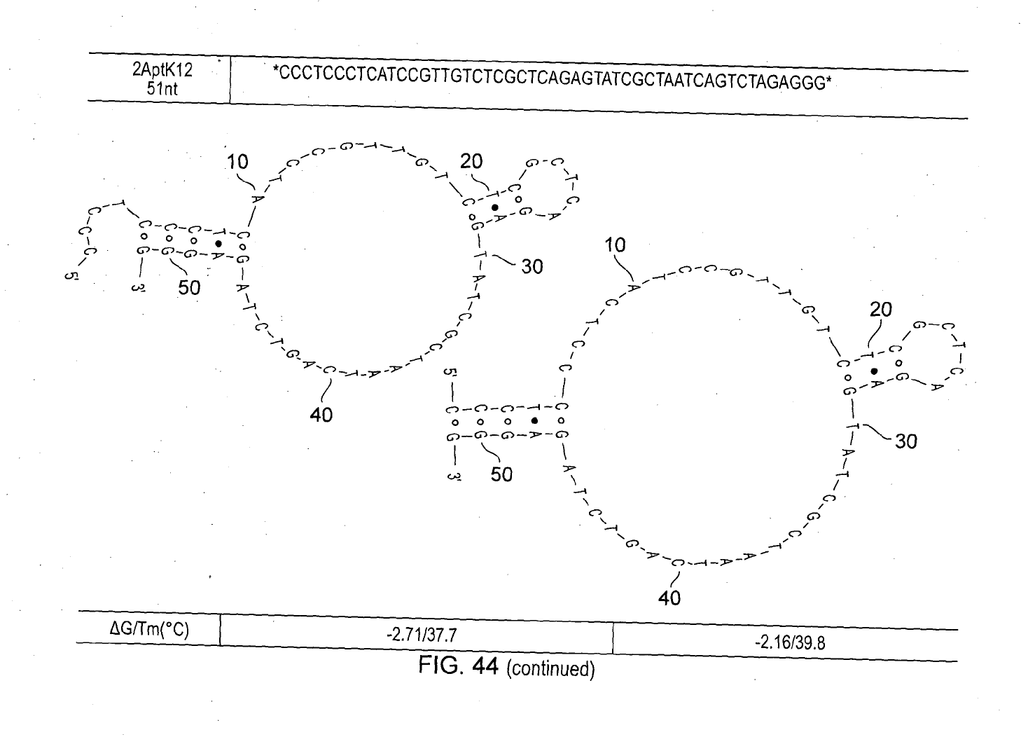

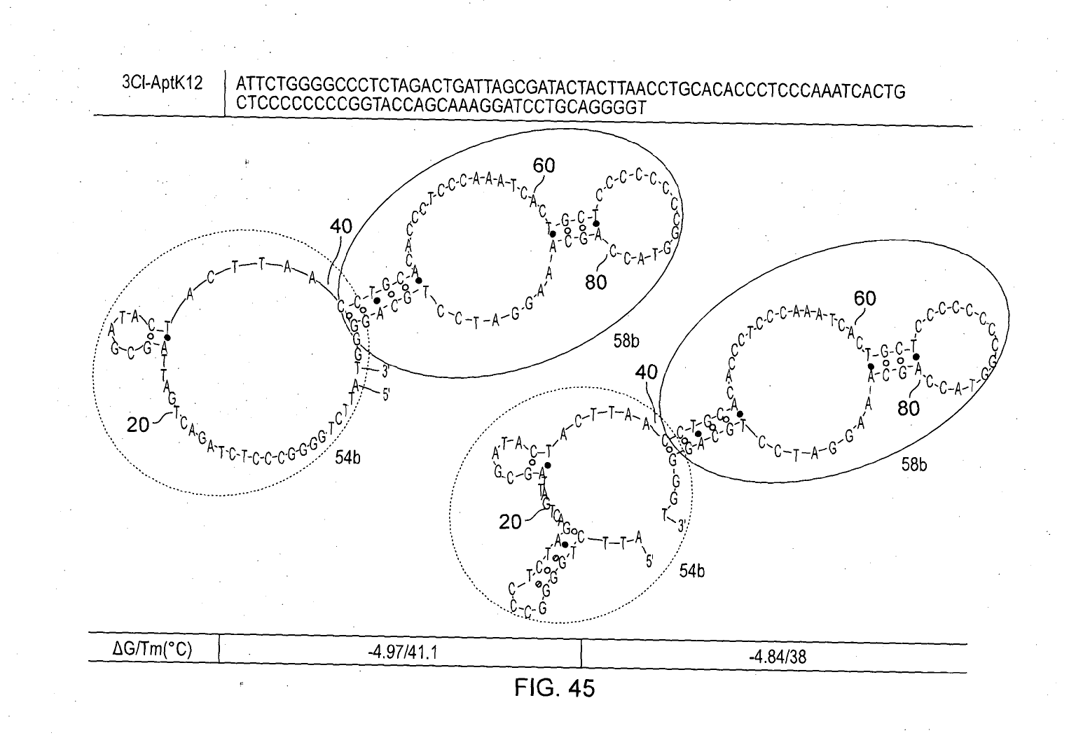

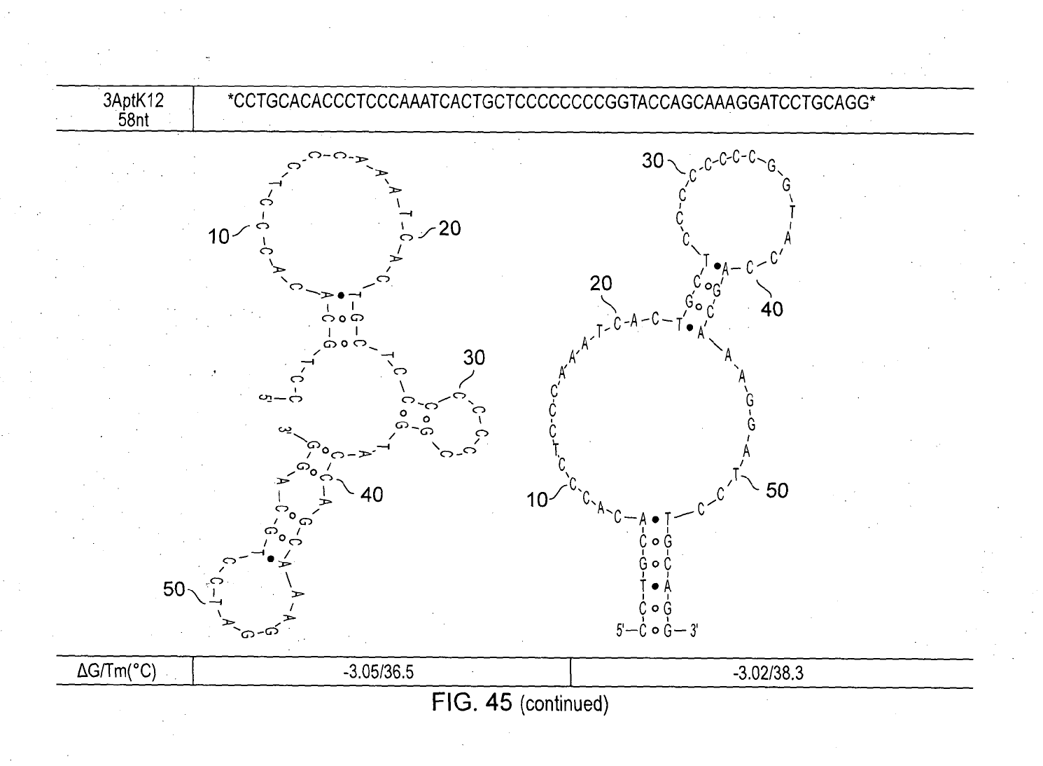

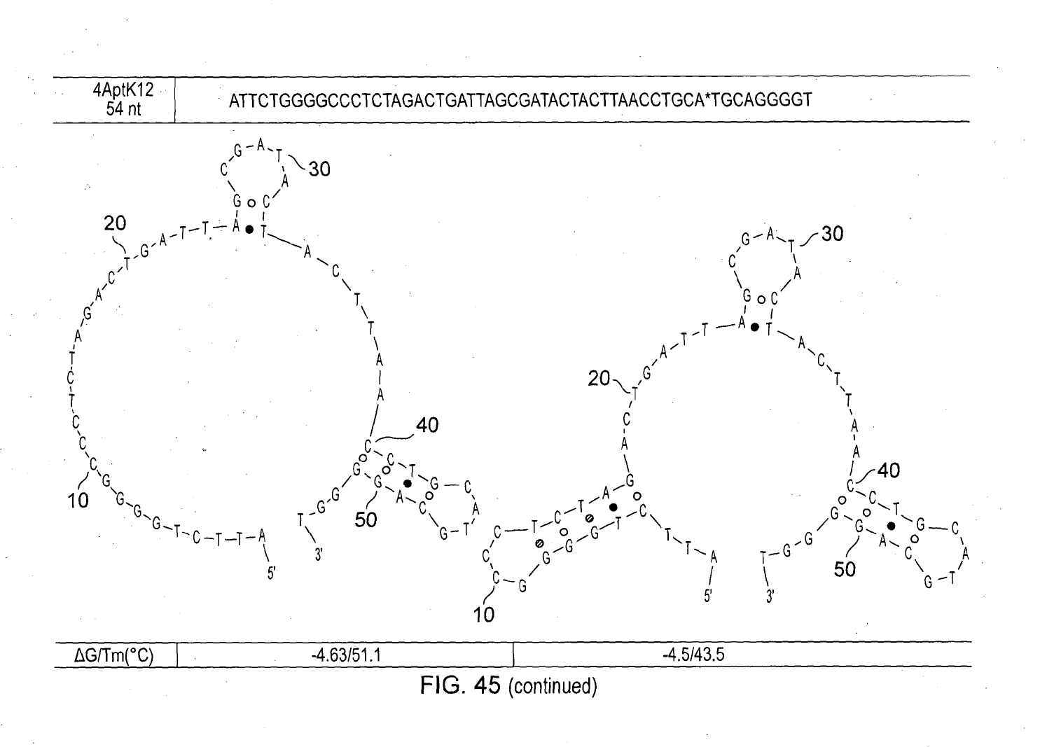

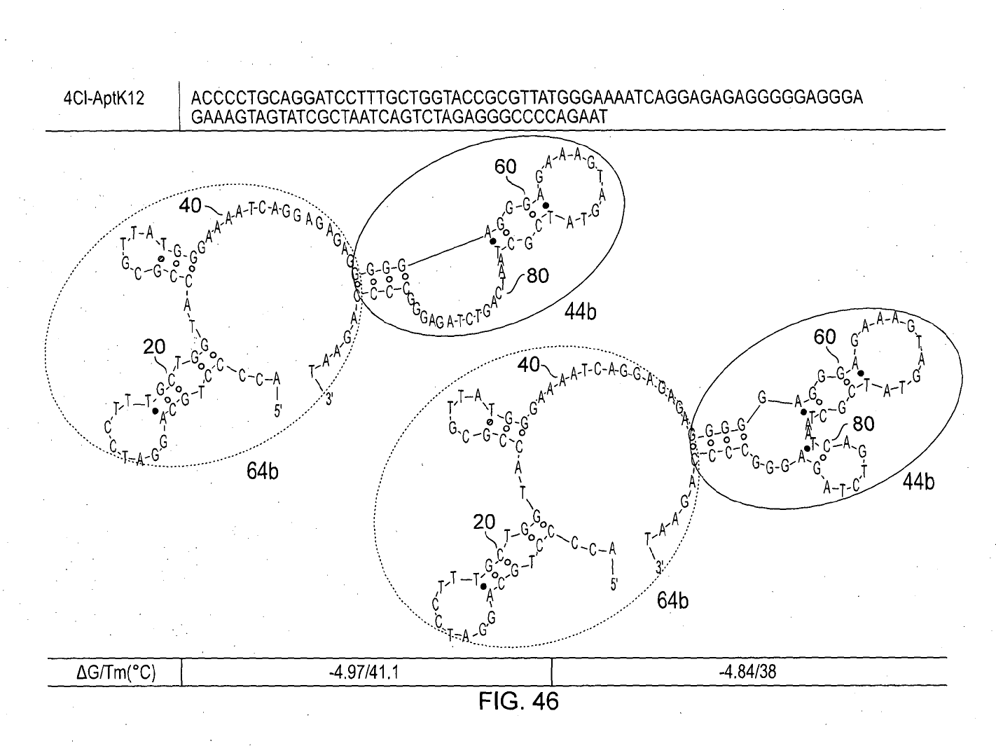

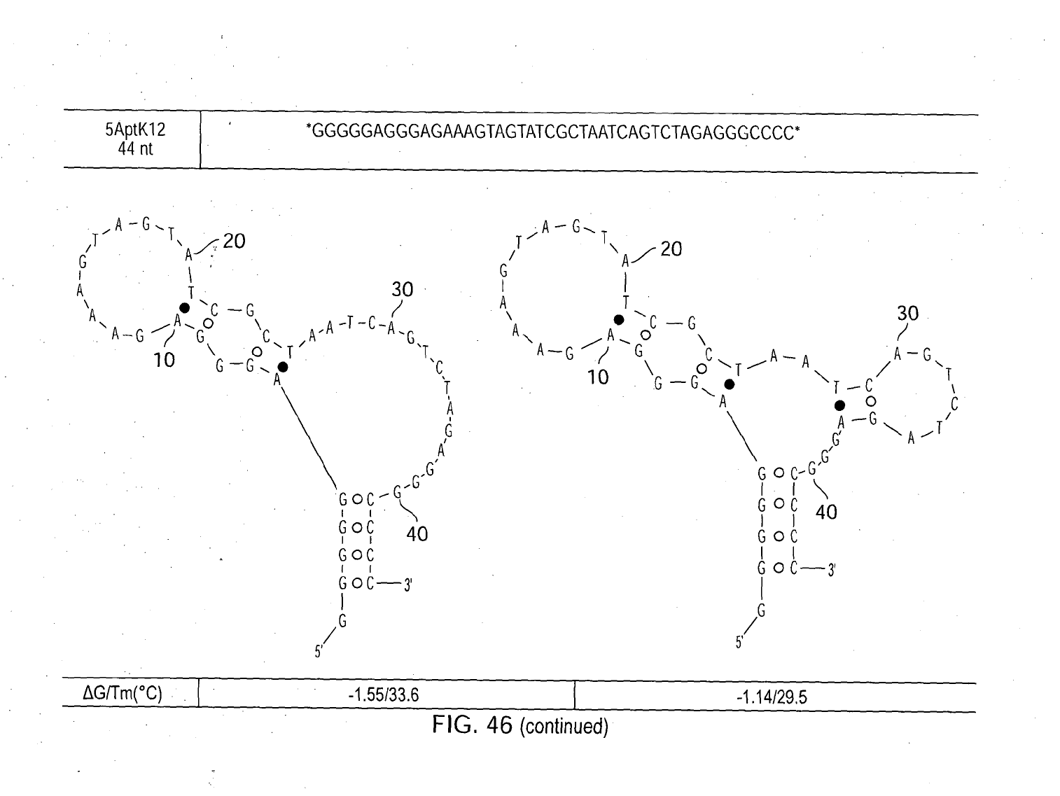

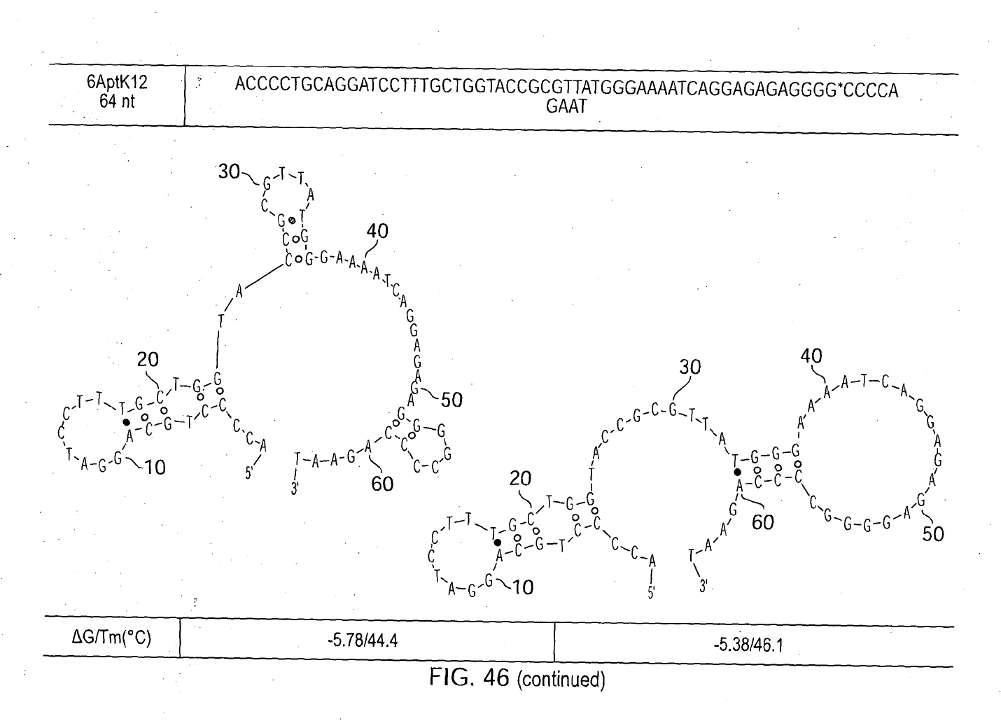

For further analysis, only the 100 nt aptamers (2CI-AptK12, 3CI-AptK12 and 4CI-AptK12) were selected. 1CI-AptK12 was left out from the further analysis because the sequence did not meet the criteria of 100 nt. In order to synthesise the aptamers the number of nucleotides had to be reduced. The aptamers were analysed using UNAFold program that gives aptamers secondary structures most likely to be formed in the binding buffer conditions. The structures are presented in FIG. 44 (2CI-AptK12), FIG. 45 (3CI-AptK12) and FIG. 46 (4CI-AptK12). Two structures for 100 nt sequences are presented on the first line and the aptamer sequences that were cut off from these 100 nt sequences are presented below. The reduced length sequences are marked by circles in the images and the isolated sequences and the secondary structures are presented for each sample. By comparing the secondary structures formed as well as the energy needed to form the structure (ΔG), four aptamers were selected and synthesised with FAM-labels. The ΔG was calculated by the UNAFold and it is defined as a change in Gibbs energy when the system undergoes a thermodynamical change. The sequences and the details of the synthesised aptamers are listed in the Table below. Only four aptamers were synthesised and two aptamers were left out. Aptamer 3AptK12 was not synthesised because the first secondary structure is not matching the original 100 nt structure. Aptamer 5AptK12 was not selected because of the very weak secondary structure (ΔG=−1.55).

TABLE
Sequences and ΔG values for the
synthesised FAM-labelled aptamers.

| Aptamer | nt | Sequence | ΔG |
|---|---|---|---|
| 1AptK12 | 54 | 5′FAM-ACCCCTGCAGGATCCTTT | −4.54 |
| | | GCTGGTACCCCGCGCGTTATTTCC | |
| | | CTGCCCCAGAAT-3′ | |
| 2AptK12 | 51 | 5′FAM-CCCTCCCTCATCCGTTGT | −2.71 |
| | | CTCGCTCAGAGTATCGCTAATCAG | |
| | | TCTAGAGGG-3′ | |
| 4AptK12 | 54 | 5′FAM-ATTCTGGGGCCCTCTAGA | −4.63 |
| | | CTGATTAGCGATACTACTTAACCT | |
| | | GCATGCAGGGGT-3′ | |
| 6AptK12 | 64 | 5′FAM-ACCCCTGCAGGATCCTTT | −5.78 |
| | | GCTGGTACCGCGTTATGGGAAAAT | |
| | | CAGGAGAGAGGGGCCCCAGAAT-3′ | |

5.3.4 E. coli K12 Binding Aptamers
5.3.4.1 Binding of the Aptamers to E. coli K12

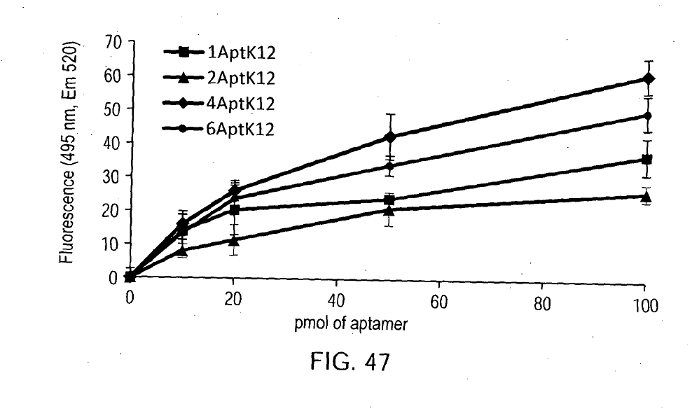

The binding of the selected aptamers to E. coli K12 was tested. The aptamers (Table above) were synthesised with a FAM-label and incubated (10, 20, 50 and 100 pmol) with E. coli K12 in triplicate. After the washes the fluorescence was measured using a plate reader and the fluorescence values are presented in FIG. 47 (this Figure is a duplication of FIG. 2). It can be seen that the values are significantly higher when the aptamers have been added (1AptK12: F=40.54, p=3.77×10⁻⁶, 2AptK12: F=28.99, p=1.77×10⁻⁵, 4AptK12: F=97.4, p=5.77×10⁻⁸, 6AptK12: F=52.46, p=1.12×10⁻⁶). The results show that the highest fluorescence value measured was for 4AptK12 and the second highest for 6AptK12 followed by 1AptK12 and the lowest was measured for 2AptK12 when 50 pmol and 100 pmol aptamers were added. The results indicate that the aptamer 4AptK12 has the strongest binding to E. coli K12. There is no significant difference between the four different aptamers when 10 pmol aptamers were added (F=2.4, p=0.14) but the rest of the samples 20 pmol (F=6.39, p=0.02), 50 pmol (F=14.49, p=1.3×10⁻³) and 100 pmol (F=31.5, p=8.83×10⁻⁵) are significantly different. The difference between the samples is bigger when more aptamers have been added. When looking at the fluorescence values of individual aptamers, it can be seen that the fluorescence is not much higher when 100 pmol of aptamers were added when compared to the samples where 50 pmol aptamers were added. The fluorescence values for 100 pmol samples are significantly higher than the 50 pmol samples of the 1AptK12 (F=17.02, p=0.01), 4AptK12 (F=14.39, p=0.02) and 6AptK12 (F=23.3, p=8.5×10⁻³) but not in sample 2AptK12 (F=2.78, p=0.17) when 100 pmol aptamers were added. These results indicate that if more than 100 pmol aptamers have been added to the samples, the fluorescence might not increase much.

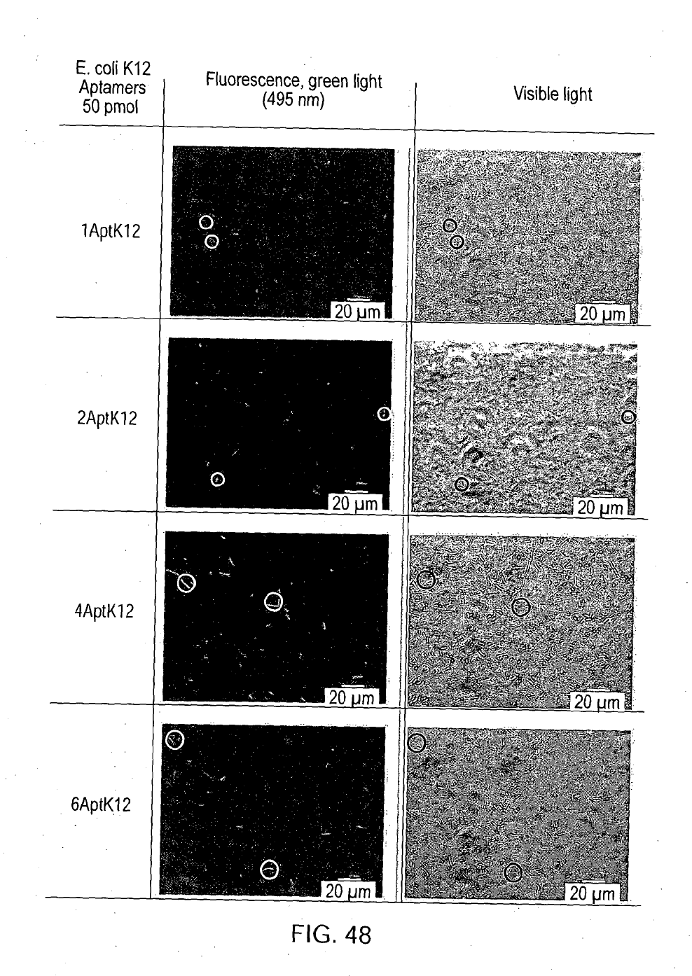

Samples were visualised under the microscope with a green fluorescence and a visible light. The results for the 50 pmol samples are shown in FIG. 48, where the fluorescence microscope images are on the left hand side and the visible light images are on the right hand side. The bacterial cells with fluorescent labelled aptamers bound to them can be seen in bright green dots. It is noticeable the sample 4AptK12 has the brightest green dots. When comparing the result for the fluorimetry readings (FIG. 47) it can be seen that the highest fluorescence was measured for this same sample. The second brightest dots are in sample 6AptK12. Also, the fluorimetry measurement gave the second strongest fluorescence for this sample. The lowest fluorescence in fluorimetry measurement was for the samples 1AptK12 and 2AptK12. This can also be seen in microscope images (FIG. 48). Interestingly, aptamer 2AptK12 has the highest ΔG value (−2.71) (Table above) and therefore the most unstable structure of these four aptamers. In the visible light image (right hand side), it can be seen that for the 2AptK12 the number of bacteria on the microscope slide is smaller than the other samples. This can also be the reason for the lower fluorescence. The strongest structure was 6AptK12 and the second strongest 4AptK12 according to ΔG values (Table above). Also the strongest fluorescence was measured (FIG. 47) and the brightest fluorescent dots can be seen (FIG. 48) in these two samples. By comparing the visible light images and the fluorescence images, it can be seen that only some of the bacterial cells have been detected with the FAM-aptamers. This has also been demonstrated previously (4.3.1.2).

### 5.3.4.2 Specificity of the Aptamers

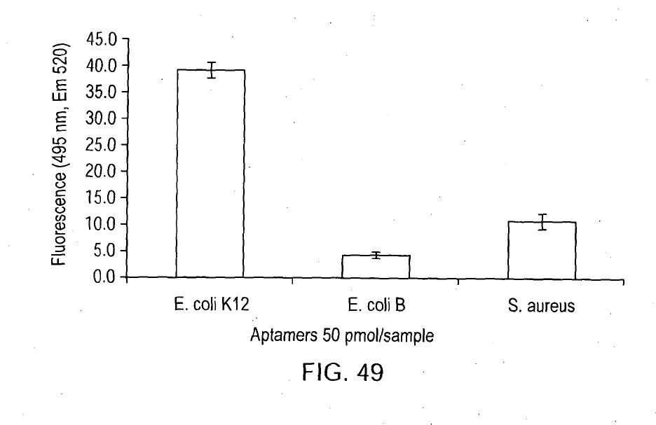

The specificity of the aptamer was tested by incubating a mixture of the aptamers 1AptK12, 2AptK12, 4AptK12 and 6AptK12 (50 pmol) with overnight grown E. coli B and S. aureus. The positive control sample used was an overnight grown E. coli K12 culture. The mixture of the aptamers was used instead of individual aptamers. After the incubation, samples were washed and the fluorescence was measured by the plate reader. The fluorescence values are presented in FIG. 49. The results showed that the fluorescence measured for E. coli K12 was significantly higher than E. coli B and S. aureus (F=626.1, p=1.08×10⁻⁷), even though some fluorescence was detected for E. coli B and S. aureus. By comparing these results to the specificity tests performed for aptamer pool 9 (4.3.4.1) it can be seen that the original aptamer amounts were lower. In that experiment, some fluorescence was also detected in E. coli B and S. aureus when 30 pmol aptamers were added. In this experiment, 50 pmol aptamers were used and higher fluorescence values were detected. This result confirms the aptamers are specific to E. coli K12 but a little binding to E. coli B and S. aureus can be detected when the aptamers concentration is high.

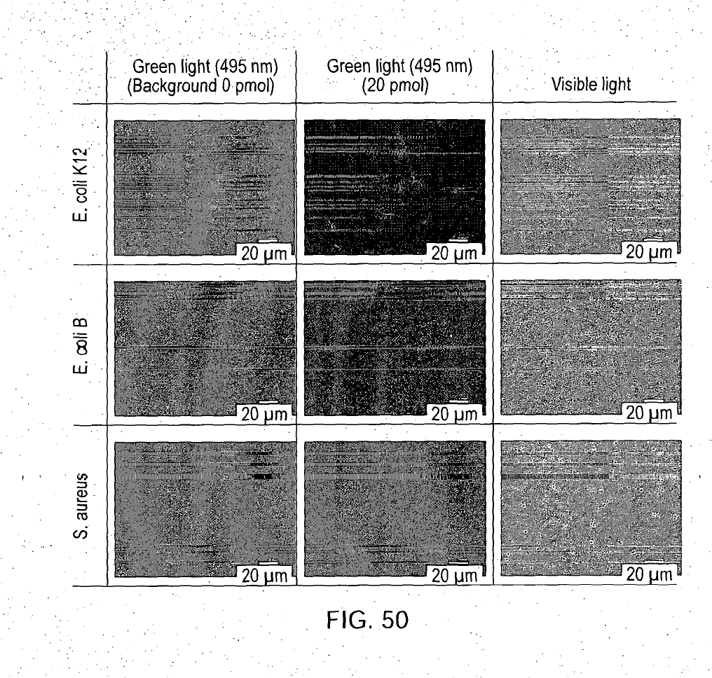

The samples were visualised under the microscope with a fluorescence and visible light. The microscope images are presented in FIG. 50. The fluorescence images for background samples (no aptamers added, 0 pmol) are on the left hand side, the images taken with fluorescence light in the middle and the visible light images on the right hand side. Bright green dots with a dark background can be detected in the positive control sample with E. coli K12. Not as bright and not as many dots can be seen in samples with E. coli B and S. aureus. This result confirms the aptamers 1AptK12, 2AptK12, 4AptK12 and 6AptK12 are specifically binding to E. coli K12 but a little binding to E. coli B and S. aureus can be seen. It can be seen on the fluorescence images that the background is darker when more fluorescence is present in the sample (E. coli K12) even though the same exposure time was used in all images (200 ms). It can be possible that the microscope is changing the lightning when not much fluorescence is available in the samples.

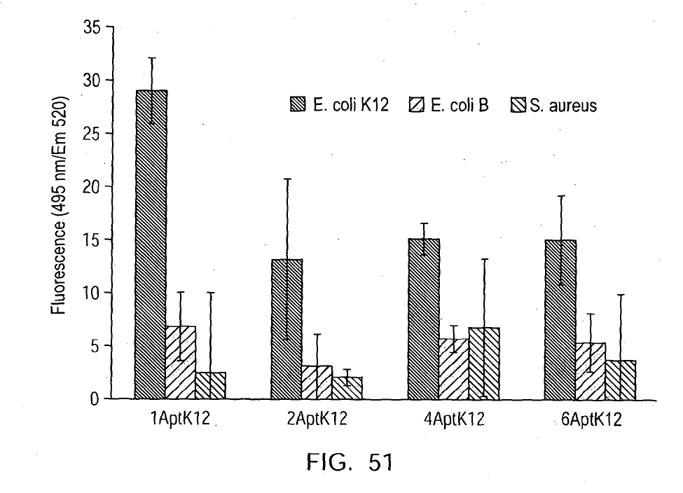

The specificity of the individual four aptamers 1AptK12, 2AptK12, 4AptK12 and 6AptK12 was tested against E. coli B and S. aureus. E. coli K12 was used as a positive control. The aptamer concentration used in this experiment was 20 pmol because this amount is showed to be enough to see the differences in the fluorescence values. Due to a problem with the plate reader no fluorescence readings could be measured with the sensitivity of 50 and therefore sensitivity 75 was used. The fluorescence values are presented in FIG. 51. The general overview of the results is that the fluorescence is higher for E. coli K12 samples than for E. coli B or S. aureus. The E. coli K12 values for all samples 1AptK12 (F=23.9, p=1.3×10⁻³), 2AptK12 (F=4.9, p=0.05), 4AptK12 (F=5.06, p=0.05) and 6AptK12 (F=5.49, p=0.04) are significantly higher than for E. coli B or S. aureus. The highest fluorescence was measured for 1AptK12 even though the binding analysis (5.3.4.1) showed the aptamer 6AptK12 has the highest and the aptamer 4AptK12 the second highest fluorescence when 20 pmol aptamers were added as seen in FIG. 47. There can be several reasons for the different results between the different measurements. The number or bacterial cells might vary as the bacterial culture is grown overnight and therefore the age of the cultures might vary. Also, as observed before, the washing steps might affect to the fluorescence readings. Sometimes more bacterial cells are washed off during the washes. The samples were looked under the microscope. Some bright dots were visible on the microscope images as seen in the images presented in FIG. 50. The fluorescence images for these samples are not shown here as the results are similar to the results when the mixture of the aptamers was incubated with the bacterial cells in FIG. 50.

### Conclusions

The aptamer pool 9 specific to E. coli K12 was selected and the binding of the aptamer pool 9 has been tested with a fluorescence based detection method. In this study aptamer pool 9, which binds specifically to E. coli K12, was cloned using a commercial cloning kit to a plasmid vector. The cloned inserts were transformed into competent cells where the insert was enriched. The plasmid vectors were extracted and sequenced. The sequenced aptamers were analysed using the aptamers theoretical secondary structures and the ΔG values. Four aptamer sequences were synthesised with a fluorescent label. The binding and the specificity of these synthesised aptamers were tested. The results show that the selected aptamers bind specifically to E. coli K12.

In this study four aptamer sequences were identified and synthesised. The binding properties of these four sequences were tested.

### Example 6

### Aptamer Detection of Escherichia coli K12 from Natural Yoghurt

### 6.1 Introduction

Aptamers were selected to bind live E. coli K12 cells. The binding properties of aptamer pool 9 were analysed and the aptamer sequences were subsequently cloned. The binding of the identified aptamers to E. coli K12 and the specificity were tested. The aptamers selected bound specifically to E. coli K12.

Aptamers have been shown to work in buffer conditions. In this study, aptamers were tested to see if they can be used to detect bacterial cells from a real food sample. The activity of the specific E. coli K12 aptamer pool 9 was first tested in tap water and the results were used to develop a detection assay that can be used for the detection of bacteria in yoghurt. The aptamers in natural probiotic yoghurt containing Lactobacillus acidophilus and

Bifidobacterium ssp. was first tested with the aptamer pool 9. Once the aptamers were cloned they were produced with fluorescent labels and then used for the detection of bacterial cells from yoghurt.

### 6.2 Methods

### 6.2.1 Aptamer Activity in Water

Aptamers selected to bind E. coli K12 have been used to detect live bacterial cells in buffer conditions. Tap water was used to see if the aptamers retained their activity in unbuffered conditions and can still be used for bacterial detection. Tap water was spiked with an excess of E. coli K12 bacterial cells. The overnight grown bacterial suspension (1 ml) was centrifuged (2.3.9.1) and the bacterial pellet was resuspended into 1 ml of tap water. The aptamers (10 pmol and 20 pmol) were added into the solution and incubated for 45 min. The control sample without the aptamers was also prepared. Samples were washed twice with BB and the fluorescence was measured by a plate reader.

6.2.2 Detection of E. coli K12 from Probiotic Yoghurt

### 6.2.2.1 Method Development

A method was developed to detect bacterial cells from food samples. Natural probiotic yoghurt that contains live cultures of L. acidophilus and Bifidobacterium ssp. was used as the food matrix. Fluorescent FAM-labelled aptamers were produced (2.3.7) and an overnight culture of E. coli K12 (10 ml) was prepared (2.3.9.1) and resuspended into 4 ml of binding buffer (BB). Yoghurt samples were prepared by mixing 3 ml of natural probiotic yoghurt and 3 ml of BB for negative samples and 3 ml of yoghurt, 2 ml of BB and 1 ml of bacterial suspension for the samples. Samples were mixed followed by centrifugation at 1500 g for 10 minutes to separate the bacteria from the yoghurt. The supernatant, containing the bacterial cells, was collected and centrifuged at 3500 g for 5 minutes. The bacterial pellet was washed twice with BB and then resuspended into 100 μl of BB containing 20 pmol of fluorescence labelled aptamers. Aptamers were bound to bacterial cell surface (2.3.9.1) and the fluorescence of the samples was read by the plate reader (2.3.9.3). The fluorescence values were analysed with the analysis of variance (ANOVA).

### 6.2.2.2 Detection

The further optimised detection method was done to detect the E. coli K12 cells from yoghurt with the aptamers. The sample sizes were reduced and less E. coli K12 bacterial cells added to make it possible to perform this experiment in microcentrifuge tubes. The FAM-labelled aptamers were produced (2.3.7) and the bacterial cells were prepared (2.3.9.1). The bacterial suspension (3 ml) was washed and re-suspended into 1500 μl of BB. The yoghurt samples were 750 μl of probiotic yoghurt and 750 μl of BB for the negative control samples. The E. coli K12 spiked yoghurt samples were 750 μl of yoghurt and 500 μl of bacterial suspension topped up with BB to the final volume of 1000 μl. The samples were mixed well and the bacteria were separated from the yoghurt by centrifuging the samples for 5 minutes at 1000 g. This centrifugation speed and time was lowered from the time and speed previously used (1500 g, 10 min) because this way more bacterial cells could be separated from the yoghurt. Supernatant, that contained bacterial cells, was collected. To recover more bacterial cells from the yoghurt mixture, an additional 200 μl of BB was added and after samples were mixed they were centrifuged as above. The supernatant was collected and added to the samples. The samples were now centrifuged at 3500 g for 5 minutes and washed twice with BB. The bacterial cells were resuspended into 100 μl of BB with 20 pmol FAM-labelled aptamers. Control samples with no aptamers were done by resuspending the cells into 100 μl BB. The yoghurt samples prepared for analysis are summarised in the Table below. The fluorescence values were read by the plate reader (2.3.9.3) and the results were analysed with the analysis of variance (ANOVA).

TABLE
Yoghurt samples.

| | | Aptamers (20 |
|---|---|---|
| Sample | E. coli K12 | pmol) |
| 0.0 | − | − |
| 0.1 | + | − |
| 1.0 | − | + |
| 1.1 | + | + |

6.2.3 Detection of E. coli K12 from Yoghurt with FAM-Labelled Aptamers

The aptamer pool 9 was cloned (5.3.1.1). Four aptamer sequences (5.3.3) were synthesised and the binding properties of these aptamers were tested (5.3.4). The yoghurt samples were prepared as described above (6.2.2.2). The bacterial cells were extracted from the yoghurt and a 20 pmol aptamer mixture contained 5 pmol of each of the cloned FAM-labelled aptamers was incubated with the samples in triplicate. Samples were incubated for 45 min and after the washes the fluorescence was measured by the plate reader. Difference between the fluorescence values was analysed with the ANOVA.

### 6.3 Results and Discussion

### 6.3.1 Aptamer Activity in Water

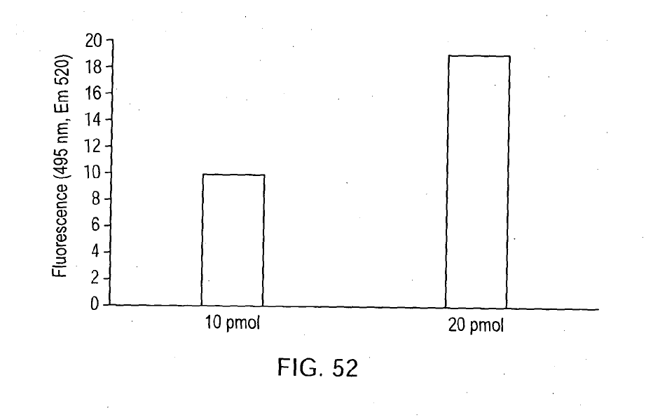

The activity of the FAM-labelled aptamer pool 9 was first tested in tap water that had been spiked with E. coli K12 bacterial cells. The fluorescence intensity values are presented in FIG. 52. Two aptamer concentrations were used (10 pmol and 20 pmol) and it can be seen in the graph that the fluorescence detected for 10 pmol sample is almost half of the fluorescence detected for the 20 pmol sample where the aptamer amount was twice as much. This result shows that the aptamers were active in water and that they can be used to detect bacterial cells in water samples.

6.3.2 Detection of E. coli K12 from Probiotic Yoghurt

### 6.3.2.1 Method Development

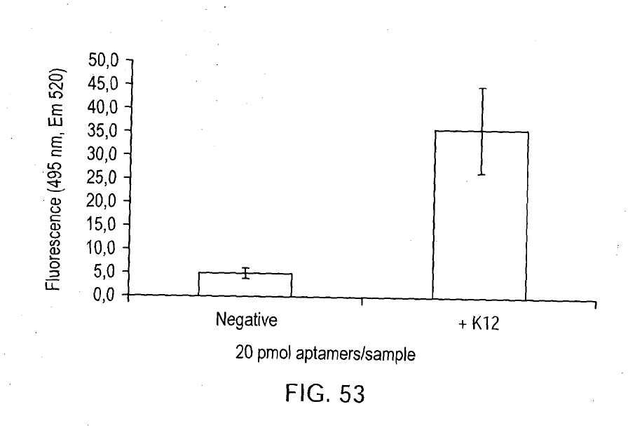

The method to detect the aptamers from natural yoghurt was tested. Yoghurt was spiked with E. coli K12 and the bacterial cells were extracted from yoghurt followed by the fluorescence aptamer detection of E. coli K12. The fluorescence values are presented in FIG. 53. It can be seen that the sample where bacterial cells have been added has significantly higher fluorescence than the sample where no E. coli K12 cells have been added (F=34.27 and p=4.2×10⁻³). The background fluorescence was not taken in to account because the control samples without the aptamers were not included in this experiment. This result, however, demonstrates that the aptamers were active and could be used to detect live bacterial cells present in yoghurt samples. The results also showed that the aptamers were not binding to the other bacteria present in yoghurt. It has previously been demonstrated that these aptamers do not bind to L. acidophilus (4.3.4.3).

6.3.2.2 Detection of the E. coli K12 from Yoghurt with Specific Aptamer Pool 9

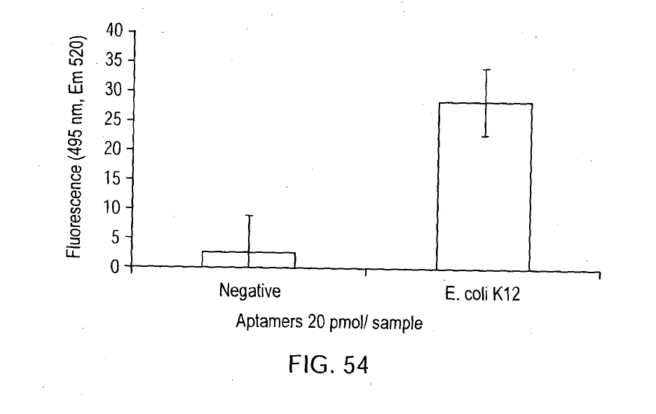

The aptamer detection of E. coli K12 from yoghurt was further optimised. The experiment was done with more control samples so that the background fluorescence could have reduced. The fluorescence values are presented in FIG. 54. It can be seen that the samples where no E. coli K12 has been added (Negative) have a significantly smaller fluorescence than for the E. coli K12 sample (F=18.6, p=0.05). This result proves that the specific aptamer pool can be used to detect live bacterial cells from yoghurt.

6.3.3 Detection of E. coli K12 from Yoghurt with FAM-Labelled Aptamers

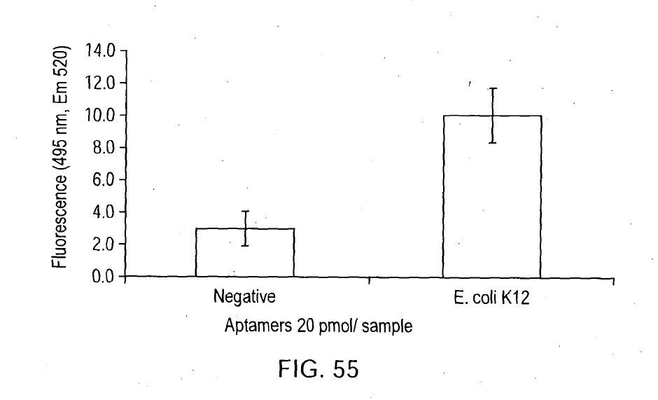

Cloned aptamers were used to detect E. coli K12 from yoghurt samples containing probiotic strains L. acidophilus and Bifidobacterium ssp. The fluorescence was measured by the plate reader and the values are presented in FIG. 55. It can be seen in the figure that the fluorescence of the sample, where E. coli K12 has been added, is significantly higher than the fluorescence in the negative sample (F=36.75, p=3.7×10⁻³). Relatively high fluorescence was measured for the negative control sample even though the fluorescence was significantly lower than the fluorescence of the E. coli K12 sample. The bacterial cells, including the bacteria in yoghurt, were extracted and it might be possible that some components from yoghurt have remained in the samples and the aptamers have bound to them. It is also possible that the unbound aptamers are not washed off properly. One possibility is that some binding to Bifidobacterium ssp. takes place and therefore some fluorescence can be detected. The results presented here show that the fluorescent-labelled aptamers can be used to detect live bacterial cells from yoghurt.

### 6.4 Conclusions

Detection of live E. coli K12 bacterial cells with specific aptamers has been previously demonstrated in buffer conditions. In this study, the binding of these specific aptamers was tested in tap water and in natural yoghurt samples. First, the activity of the aptamer pool in water was tested and the results demonstrated that the aptamers did not lose their activity in water. The aptamer activity was also tested in bacterial cells extracted from yoghurt where the aptamers can be used to detect live bacterial cells. Once the aptamers were cloned the detection method developed was performed with a mixture of four individual aptamers. The detection of the E. coli K12 bacterial cells in yoghurt was successful with the cloned aptamers.

Aptamers were used to detect bacterial cells from water and yoghurt. Thus the aptamers can be used in a new type of detection method for food poisoning bacteria. In this study non-pathogenic E. coli K12 was used as an example strain but aptamers can also be used in a detection of pathogens. Aptamers can be used to detect the bacterial cells in different types of food matrices, for example in solid matrices such as meat or cheese. Aptamers could possibly be added on the surface of the meat and the non-binding aptamers could be washed off. The fluorescence can then be measured or visualised.

### Example 7

### Selection of the Aptamers Against Pathogenic Bacteria

### 7.1 Introduction

Aptamers have been shown herein to be a tool in the development of rapid detection methods for bacteria such as, food-borne pathogens. The selection method for aptamers was developed and the aptamers were selected to bind to non-pathogenic E. coli K12 live bacterial cells. The aptamers were then cloned, sequenced and the binding and the specificity of these selected sequences were tested by using a method based on fluorescence.

The aptamer selection method was applied to the food-borne pathogenic bacteria. The aptamers were selected to bind to two different pathogenic E. coli strains including O157, three Listeria strains (L. innocua and two species of L. monocytogenes) and two Salmonella strains (S. typhimurium and S. enteritidis). The aptamers against pathogenic E. coli were selected from an existing pool of E. coli K12 binding aptamers while the selection process that was previously described was used to select the aptamers against Listeria and Salmonella. From these aptamers two pools having the best characteristics were selected for further analysis. As a result of this study, three aptamer sequences for E. coli O157 and three sequences for S. typhimurium were identified and the binding was tested. Some of the sequences showed specific binding and good affinity against their target bacteria.

### 7.2 Methods

### 7.2.1 Aptamers Selection

A DNA library was produced as described before (2.2.3) and the aptamers were selected to bind to pathogenic bacterial strains. The selection of the aptamers was done following the protocol (2.3.6) with one exception; the counter selection was not performed. The counter selection was left out in order to see if the counter selection is necessary in terms of the specificity of the aptamers but also for time saving reasons.

7.2.1.1 Aptamers Against Escherichia coli 496 and O157 497

Aptamers were selected to bind to two strains of pathogenic E. coli from pool 9 of E. coli K12 binding aptamers. It is possible the aptamer pool 9 binds to some structures on the surface of E. coli K12 that are the same as on the surface of the pathogenic E. coli strains. Selection was done as a normal aptamer selection (2.3.6) except that the selection process was only performed once. The selection and the counter selection were done before as described (3.3.2). After the incubation the samples were washed three times. The aptamers were collected and amplified by PCR (2.3.1) and the samples were separated on agarose gel (2.3.2).

7.2.1.2 Aptamers Against Listeria Innocua and Listeria monocytogenes

The aptamers were selected from the random DNA library to bind to L. innocua 17, L. monocytogenes 489 and L. monocytogenes 490 as previously described (2.3.6), except the counter selection was not performed. The aptamers were selected for all three strains until aptamer pool 7. The only successfully selected aptamer pool 7 was for L. monocytogenes 490. This sample was divided in two tubes and the aptamers were further selected in duplicate. To increase the PCR yield the template was added to the reaction in volume 1.5 μl instead of 1 μl. The aptamers were collected and amplified by PCR (2.3.1) and the samples were separated on agarose gel (2.3.2).

7.2.1.3 Aptamers Against Salmonella typhimurium and Salmonella enteritidis

The aptamers were selected from a random DNA library to bind to S. typhimurium 223 and S. enteritidis 1152 following the selection protocol (2.3.6). The selection without the counter selection steps was repeated nine times. After each selection round the aptamers were collected and amplified by PCR (2.3.1) and the samples were separated on agarose gel (2.3.2).

### 7.2.2 Fluorimetry Detection of the Pathogen Binding Aptamer Pools

The aptamer pools were fluorescent (FAM) labelled (2.3.7) and the binding of the aptamers was tested by the fluorimetry analysis (2.3.9.3). Four PCR products were mixed together, purified and used in the binding reaction. The binding was tested for the aptamers that were selected against E. coli 496, E. coli 497, L. monocytogenes 490, S. typhimurium 223 and S. enteritidis 1152. The binding of the previously selected E. coli K12 aptamers (3.3.2) was tested parallel. The DNA concentration measurement was not used in this study but the fluorescence of the aptamers was measured before they were mixed with the samples.

### 7.2.3 Cloning of the Aptamers

To find out the specific sequences of the aptamers the ninth aptamer pools against E. coli O157 497 and S. typhimurium 223 were selected for cloning. The pGEM-T Easy vector cloning was done as previously described (2.3.12.1) and the same protocol was followed as for the cloning of E. coli K12 aptamers (5.2.1).

### 7.2.3.1 Ligation and Transformation

Aptamer pool 9 against E. coli O157 497 and S. typhimurium 223 were ligated into the plasmid vector (2.3.12.1). In this study, three different insert:vector ratios were used (5.2.1.1) for the ligation as in the ligation for the E. coli K12 aptamers. The components for the reaction are presented in Table 5.1. From the indicator plates, five positive white and one negative blue colony were randomly selected and streaked on new indicator plates. This was done for both, E. coli and S. typhimurium samples. The plates were incubated overnight.

### 7.2.3.2 PCR Analysis for Positive Colonies

The overnight grown colonies were analysed. It has previously been demonstrated that the colour selection is not reliable method for selecting the positive colonies. The PCR analysis was demonstrated to be a fast, easy and reliable way of analysing the positive colonies (5.3.1.2). The PCR analysis of the samples was performed for all of the colonies on the indicator plates (2.3.12.2; 5.3.1.2). Among the PCR control a negative control, that contained the PCR primers and a colony with a control insert in it (positive control for cloning), was done.

### 7.2.4 Sequencing of Cloned Vectors and Aptamer Analysis

The aptamer sequences were determined by sequencing the plasmid vectors with inserted aptamers. The colonies that were showed to be positive in PCR analysis were inoculated into LB-media and the plasmid was purified from the overnight grown culture (2.3.12.4). Six plasmid DNA samples (30 μl), three of each strain, were sequenced (2.3.12.6). The 100 bases long aptamer sequences were identified from the vector sequence.

Aptamer secondary structures make the binding of the aptamer to their target possible. The structures were analysed as previously described (2.3.12.7). Three aptamers for E. coli O157 497 and three aptamers for S. typhimurium 223 were selected and synthesised with a fluorescent FAM label. These aptamers were selected because of their matching structure to the original 100 nucleotides secondary structure. The energy needed to break down the structure (ΔG) that also represents the strength of the structure was taken into account when selecting the aptamers to be synthesised.

### 7.2.5 Binding of the Cloned Aptamers

Three cloned and synthesised fluorescent labelled aptamers for both strains E. coli O157 497 (1Apt497, 2Apt497 and 4Apt497) and S. typhimurium 223 (2Apt223, 3Apt223 and 5Apt223) were tested. The aptamers (10 pmol, 20 pmol and 50 pmol) were incubated with bacterial suspension in triplicate and the fluorescence was analysed by fluorimetry and by visualising the samples under the fluorescence microscope (2.3.9). The fluorescence values were analysed using the statistic test ANOVA.

7.2.5.1 E. coli O157 Aptamers

The binding of the fluorescent-labelled aptamers 1Apt497, 2Apt497 and 4Apt497 against E. coli O157 497 was tested as described above (7.2.5).

7.2.5.2 Binding of E. coli O157 Aptamers to E. coli K12

The aptamers to bind to E. coli O157 497 were selected from a pool of E. coli K12 binding aptamers. The binding of these aptamers against E. coli K12 was tested by incubating 20 pmol of the aptamer 1Apt497, 2Apt497 and 5AptK12 with E. coli K12 bacterial cells and then measuring the binding by fluorimetry test (2.3.9.3). E. coli K12 specific aptamer 4Apt497 was used as a positive control. The fluorescence was measured with a sensitivity of 75 instead of 50. The fluorescence values were analysed using the statistic test ANOVA. The samples were then visualised and the images were taken under the fluorescence microscope with a 60× magnification (2.3.9.2).

7.2.5.3 S. typhimurium Binding Aptamers

The binding of the fluorescent-labelled aptamers 2Apt223, 3Apt223 and 5Apt223 against S. typhimurium 223 was tested as described above (7.2.5).

7.2.5.4 Specificity of the S. typhimurium Binding Aptamers

The specificity of the S. typhimurium binding aptamers 2Apt223, 3Apt223 and 5Apt223 was tested. The aptamers (20 pmol) were incubated with E. coli K12, L. plantarum, S. enteritidis and S. typhimurium 223 (2.3.9.1) in triplicate. After the incubation, the samples were washed and the fluorescence was measured by the plate reader (2.3.9.3) and the samples were visualised under the fluorescence microscope (2.3.9.2). The fluorescence values were analysed using the statistic test ANOVA.

### 7.3 Results and Discussion

### 7.3.1 Aptamers Selection

The aptamers were selected from the random DNA library that has previously been created (3.3.1). An agarose gel image of the DNA library can be seen in FIG. 15.

7.3.1.1 Aptamers Against Escherichia coli 496 and O157 497

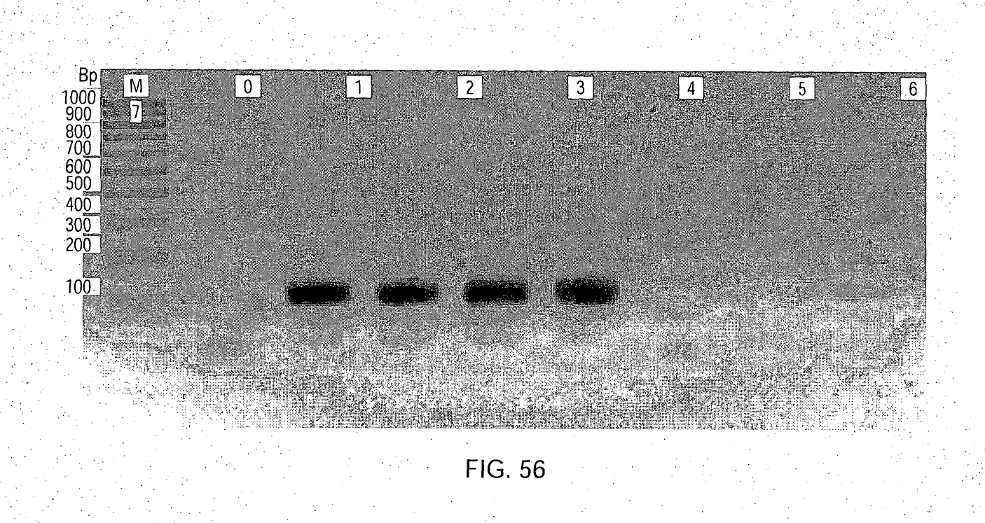

Aptamer pool 9 was previously selected to bind to E. coli K12 (3.3.2) and the binding of this pool to its target has previously been shown (3.3.4; 4.3.1). In this study, the aptamers were selected to bind to pathogenic E. coli 496 and E. coli O157 497 from the E. coli K12 binding aptamer pool 9. The agarose gel image is presented in FIG. 56. It can be seen that all the aptamer pools (1-2: E. coli 496 and 3-4: E. coli O157 497) have strong bands. The amplification of these samples indicates that the aptamers have bound to the pathogenic strains of E. coli. The bacterial control samples are in lanes 5 and 6. No amplification can be detected in these samples as expected. A faint band can be seen in the DNA control sample in lane 7. This indicates some aptamers that are not necessarily binding to E. coli are remaining in the tube after the washes.

7.3.1.2 Aptamers Against Listeria Innocua and Listeria monocytogenes

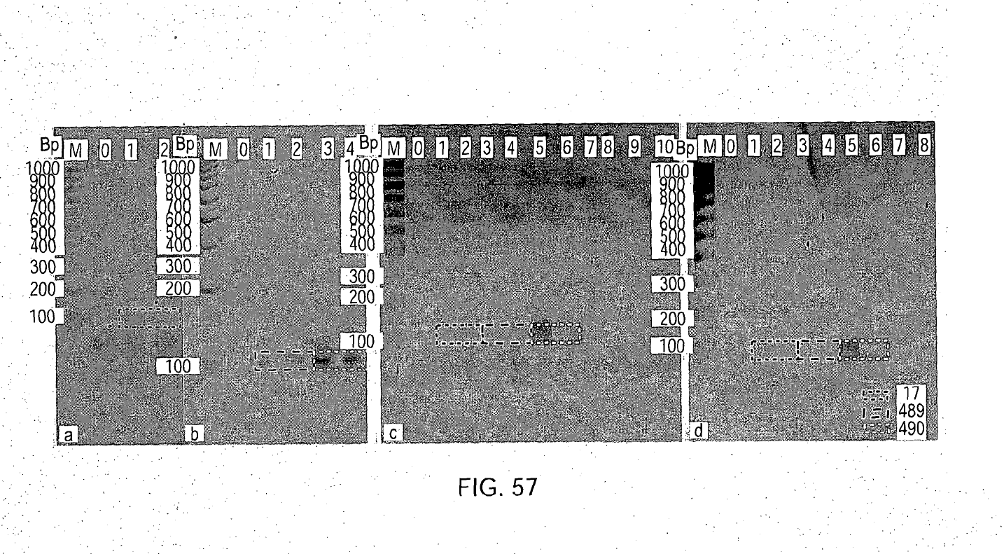

Aptamers were selected to bind to three different Listeria strains: L. innocua 17, L. monocytogenes 489 and L. monocytogenes 490. Only seven rounds of selection were repeated for L. innocua 17 and L. monocytogenes 489, because no PCR amplification could be seen after the sixth round of selection. For L. monocytogenes 490 nine rounds of selection were performed. After each round the PCR products were separated on agarose gel to see the 100 bp product. The selection of the aptamer pools 1, 2, 3 and 4 was successful even though the PCR resulted only in faint bands on agarose gels. The gel images are not presented here. In FIG. 57 is an agarose gel images of the aptamer pools 5, 6 and 7. It can be seen that the aptamers for L. innocua 17 in lanes 1 and 2 on gel 7.2a, 7.2c and 7.2d (black boxes) have faint bands. The bands can also be seen in the L. monocytogenes 489 aptamers in lanes 1 and 2 on gel 7.2b and lanes 3 and 4 on gel 7.2c and 7.2d (grey boxes). Aptamers for L. monocytogenes 490 in lanes 3 and 4 on gel 7.2b (white boxes) have the strongest bands. For aptamer pool 6 and 7 only one of the 490 aptamers appears to have a strong band on the gel (lane 5 on gel 7.2c and 7.2d). This indicates there is more aptamer binding to the bacterial cells in this sample comparing to two other Listeria strains. This strong band was extracted, divided into two and used for further selection. The PCR control, where no aptamer template was added, is in lane 0 on both of the gels 7.2a and 7.2b. The bacterial control and DNA control resulted in clear bands but the results are not seen in the gel images shown in FIG. 57.

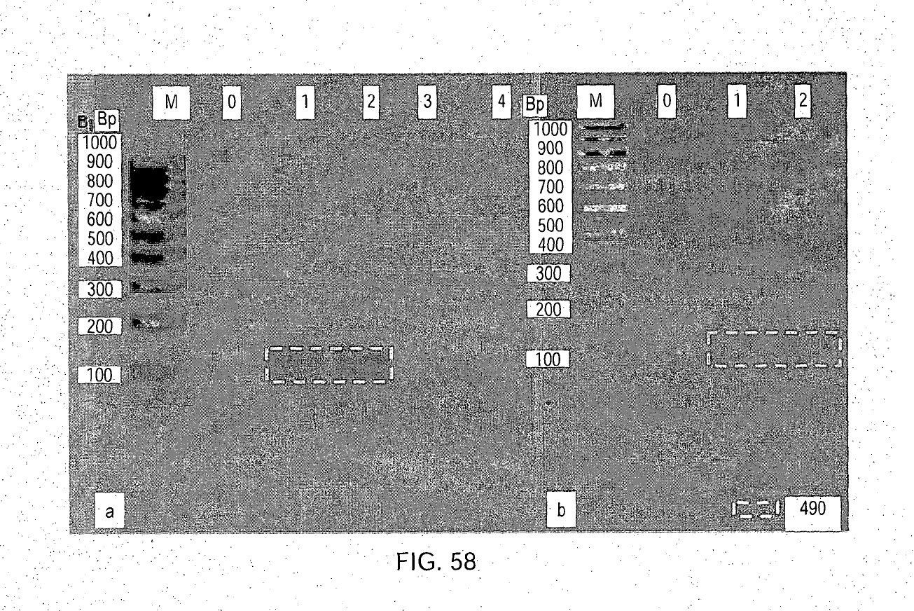

After the poor amplification of the aptamers against L. innocua 17 and L. monocytogenes 489 that can be seen in FIG. 57 the further selection was only performed for L. monocytogenes 490. The agarose gels for aptamer pools 8 and 9 are shown in FIG. 58. The amplification products are marked with white boxes in lanes 1 and 2. The bacterial and DNA control samples are on gel a (FIG. 58a) in lanes 3 and 4. These control samples were also performed for the selection round 9 but the results are not presented on the agarose gel b (FIG. 58b).

7.3.1.3 Aptamers Against Salmonella typhimurium and Salmonella enteritidis

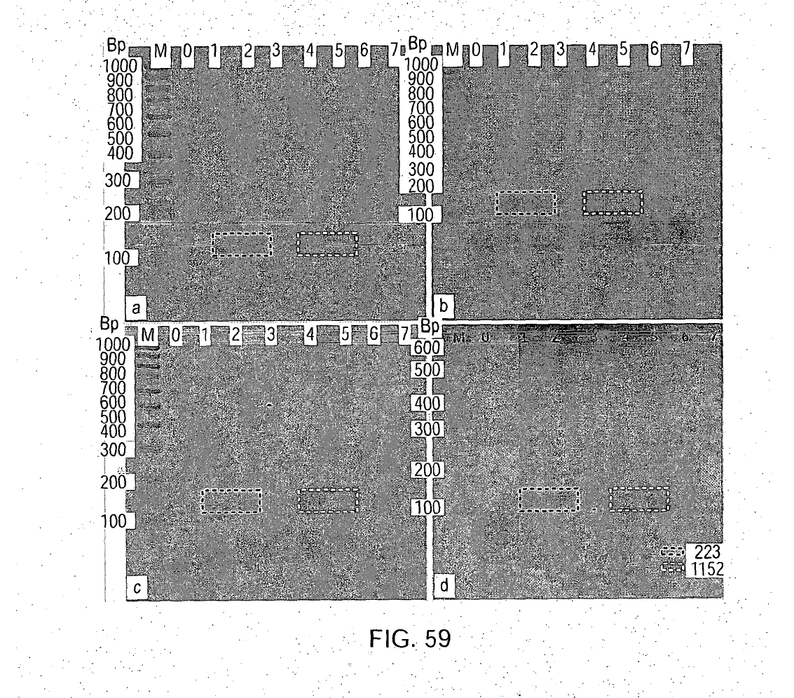

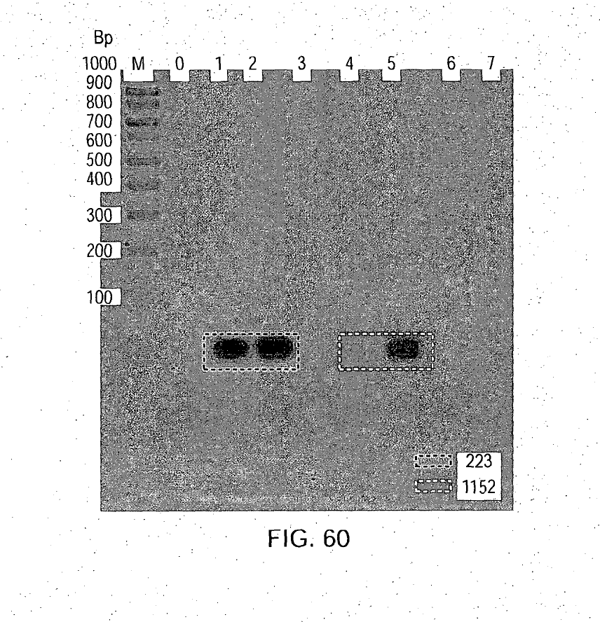

Aptamers were selected to bind to S. typhimurium 223 and S. enteritidis 1152. Nine rounds of selection were performed. The agarose gels of aptamer pool 1, 2, 3 and 4 are shown in FIG. 59 and pool 5 in FIG. 60. The aptamers for S. typhimurium 223 are in lanes 1 and 2 (black box) and the bacterial control sample is in lane 3. The aptamers for S. enteritidis 1152 are in lanes 4 and 5 (white box), and the bacterial control is in lane 6. The DNA control sample where no bacterial cells were added is in lane 7. It can be seen in FIG. 59 that the bands for the S. typhimurium 223 aptamers are slightly more intense than the bands for the S. enteritidis 1152 aptamers. The higher PCR yield may indicate that from the original DNA library more aptamers have bound to S. typhimurium 223 than to S. enteritidis 1152. In FIG. 60 the results for pool 5 are shown. The same trend is seen for this pool with S. typhimurium 223 bands being stronger than the bands for S. enteritidis 1152 except for the aptamer pool for S. enteritidis 1152 in lane 5 (FIG. 60). This pool show a similar intensity as the S. typhimurium 223 pools but stronger than the pool in lane 4. The reason for this can be that more aptamers have bound to the bacterial cells and therefore the template concentration in PCR is higher. The PCR control samples (0) appear to be clear in all gels. No amplification can be seen in bacterial control samples (lanes 3 and 6). This result indicates the bacterial cells do not have the binding sites for the primers. The clear DNA control sample shows there were no free aptamers remaining in the solution or binding to the tube wall after the washes.

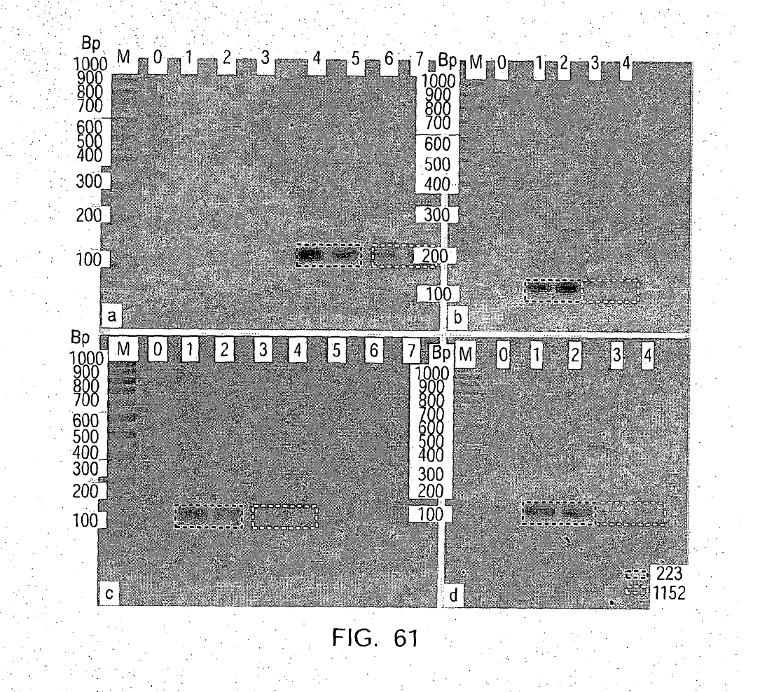

The aptamer pools 6, 7, 8 and 9 are shown in FIG. 61. The aptamers for S. typhimurium 223 are marked with black boxes and for S. enteritidis 1152 are in white boxes. The results show that the aptamer pools for S. typhimurium 223 (black boxes) have stronger bands than the S. enteritidis 1152 for all the pools (white boxes). The PCR control samples (lanes 0) are clear as expected. Bacterial and DNA control samples were done for all selection rounds but can only be seen on gel 7.6a in lanes 1-3 and on gel 7.6c in lanes 5-7. No amplification was seen in the bacterial control samples. The DNA control sample on gel 7.6c in lane 7 has a faint band. The samples were transferred into new fresh tubes after the incubation in order to avoid collecting the aptamers that are binding the tube wall. Even though this step was done; some of the molecules have stayed in the solution where no bacterial cells were added (DNA control)

### 7.3.2 Fluorimetry Detection of the Pathogen Binding Aptamer Pools

The aptamer pools were fluorescent (FAM) labelled (2.3.7) and the binding was detected by the fluorimetry analysis. Instead of measuring the DNA concentration of the aptamers, in this study, the fluorescence values of the aptamers were measured before the aptamers were added to the bacterial cells.

Aptamer pool 9 was previously selected for E. coli K12 (3.3.2) and in this study aptamers were selected against E. coli 496, E. coli 497, L. monocytogenes 490, S. typhimurium 223 and S. enteritidis 1152 and the PCR products were separated on agarose gels. The binding of the aptamers was tested with the previously developed fluorimetry detection assay (4.2.1.3). The binding of the E. coli K12 aptamers to their target has previously been demonstrated (3.3.4; 4.3.1) and this aptamer pool was used as a positive control in this experiment. The fluorescence values of the pathogen samples are shown in the Table below. Two samples, as seen in agarose gel images (FIG. 56-61), of each bacterial strain were performed in duplicate. Only one positive control (E. coli K12) was performed. The fluorescence values of the aptamer pools before the binding reaction can be seen in row 0. After the incubation with the bacterial cell the samples were centrifuged and washed followed by the fluorescence measurement of the supernatants (1^{st} wash, 2^{nd} wash and 3^{rd} wash). It can be seen that the values are decreasing after each washes. This indicates there are less unbound aptamers in the solution. After the washes the samples were resuspended into the buffer and the fluorescence was measures (Samples). The results show that no fluorescence can be detected for E. coli 496, L. monocytogenes 490 and S. enteritidis 1152 while some binding can be detected for E. coli O157 497 and S. typhimurium 223. It can be seen that the fluorescence values of the pathogen aptamers (E. coli 496 and 497, L. monocytogenes 490, Salmonella 223 and 1152) are much lower than the values of the E. coli K12 binding aptamers. The higher the fluorescence the more aptamers have bound. These results indicate there is not much aptamers binding to the pathogen strains. The fluorescence values measured after the 1^{st} wash are very high. When compared to the values in row 0, it can be seen that the most of the aptamers have not bound to the bacterial cells and were washed off. It is possible that there is not enough binding sites on the bacterial cell surface or the aptamers anneal back together to their complementary strand and therefore are not able to form the structure that is needed for their binding to the target. The difference between the fluorescence values (row 0) between different samples is also noticeable. This is mainly caused by the variable concentrations of the aptamers that were mixed with the cells. The difference can already be seen on the agarose gel images where the intensity of the bands varies. For example S. enteritidis 1152 aptamers did not yield to a strong PCR product (FIG. 61) and therefore the fluorescence is also very small compared to S. typhimurium 223 aptamers. S. typhimurium 223 aptamers also have a strong band on a gel (FIG. 61c) and the fluorescence is much higher in purified aptamers (see Table below, row 0). The aptamer concentrations could have been optimised to this experiment by producing more aptamers by PCR. This would have required several PCR reactions as in a normal binding reaction, when the aptamer concentration is higher, eight PCR reactions were needed. Two aptamer pools were decided to be chosen for further experiments because of the limited time and limited handling resources. The aptamer pool 9 against E. coli O157 497 and the aptamer pool 9 against S. typhimurium 223 were shown to have the best binding properties according to the results presented here and were therefore chosen for further experiments.

TABLE
Fluorescence values for E. coli K12 (n = 4), E. coli 496, E. coli 497, L. monocytogenes
490, S. typhimurium 223 and S. enteritidis 1152 (n = 2). The values are corrected for background.

| | | | | | | L. | | S. | | S. | |
|---|---|---|---|---|---|---|---|---|---|---|---|
| Fluorescence | | E. coli | | E. coli | | monocytogenes | | typhimurium | | enteritidis | |
| (495 nm, Em | E. coli | 496 | | 497 | | 49 | | 22 | | 115 | |
| 520) | K12 | 1 | 2 | 1 | 2 | 1 | 2 | 1 | 2 | 1 | 2 |
| 0 | 2900 | 2400 | 2500 | 2200 | 2400 | 400 | 400 | 1300 | 1300 | 439 | 539 |
| 1^{st} wash | 2450 | 2200 | 2200 | 2000 | 2100 | 170 | 240 | 840 | 970 | 170 | 244 |
| 2^{nd} wash | 56 | 90 | 70 | 73 | 82 | 31 | 21 | 161 | 210 | 31 | 21 |
| 3^{rd} wash | 11 | 6 | 7 | 9 | 6 | 3 | 1 | 17 | 18 | 3 | 1 |
| Samples | 20 | 0 | 0 | 1.5 | 0.5 | 0 | 0 | 1.5 | 0 | 0 | 0 |

### 7.3.3 Cloning of the Aptamers

### 7.3.3.1 Ligation and Transformation

The aptamer pool 9 against E. coli O157 and the aptamer pool 9 against S. typhimurium 223 were shown to have the best binding properties and were therefore chosen for further experiments. These pools of aptamers with unknown sequences were cloned and sequenced to determine the nucleotide sequence. Aptamer pools 9 were amplified by PCR and cloned with pGEM-T Easy vector and JM109 competent cells. The colonies on indicator plates, containing ampicillin, X-Gal and IPTG were counted and a number of positive (white) and negative (blue) colonies is presented in the Table below. The colonies are only growing on the plates due the ampicillin resistance of the bacteria that has been gained by a successful ligation of the insert into the vector. The background is representing the number of bacteria without the vector growing on the plate and the positive control sample is representing a successful transformation into the competent cells. For both pathogen strains, six white colonies were selected and streaked on new selective plates. After an overnight incubation on selective plates all of these colonies that were white on the first plate were expressing the blue colour (CI1s-CI6s, CI1e, CI3e-CI6e), expect one sample that remained white (CI2e). Sometimes the blue colour may be expressed even though the insert has been ligated into the vector, as previously discussed (5.3.1.1).

TABLE
Cloned colonies of E. coli O157 and
S. typhimurium 223 on indicator plates.

| Insert:Vector | | Blue | White | |
|---|---|---|---|---|
| molar ratio | | (negative) | (positive) | Total |
| Background | | 8 | — | 8 |
| Positive control | | 3 | 47 | 50 |
| E. coli O157 | 1:4 | 20 | 5 | 25 |
| | 1:2 | ∞ | ∞ | ∞ |
| | 1:1 | 241 | 18 | 259 |
| S. typhimurium | 1:4 | 95 | 26 | 119 |
| | 1:2 | 300 | 64 | 364 |
| | 1:1 | 141 | 18 | 159 |

### 7.3.3.2 PCR Analysis for Positive Colonies

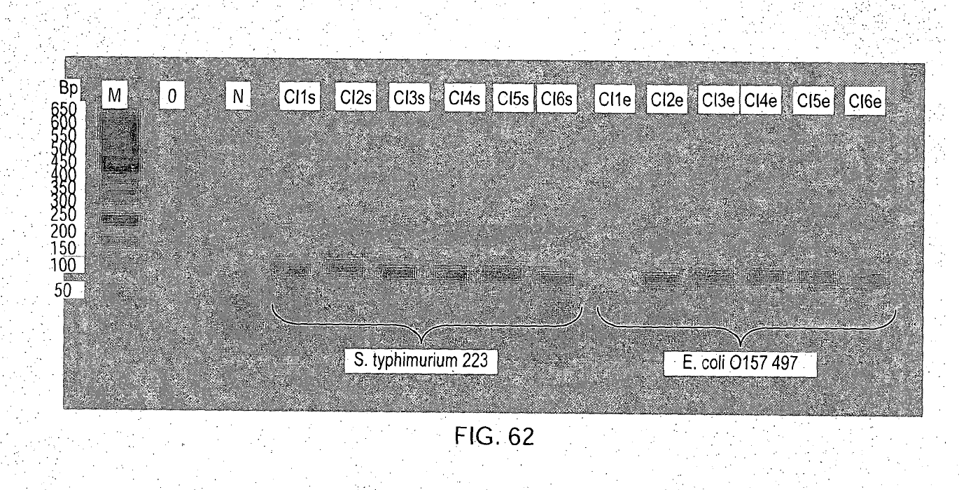

It was described above that some colonies without the vector and the insert may be growing on the indicator plates. It was also previously demonstrated that the colour selection is not reliable way of selecting the positive colonies (5.3.1.2). The colonies from the selective plates were therefore analysed by PCR to see if the insertion of the aptamers into the vector has been successful. Due the use of the aptamer primers PR1 and PR2, only the colonies with the aptamer sequences and the primer binding sites are amplified in PCR. This results in a 100 bp PCR products. The agarose gel of the PCR products is shown in FIG. 62. On the gel the aptamers against S. typhimurium 223 are in the lanes CI1s-CI6s and against E. coli O157 497 in the lanes CI1e-CI6e. All of the samples have a 100 bp bands on the gel except sample CI1e. This result indicates that all of the colonies having the 100 bp band have the aptamer insert in their plasmid. Sample CI1e was not amplified in PCR and therefore this colony did not contain the plasmid vector with an aptamer ligated in it. The positive colonies (CI1 s-CI6s and CI2e-CI6e) were selected for the further analysis. On the gel in lane 0 is the PCR control and in lane N is the negative control. Both samples are clear as expected. The negative control was amplified by using a colony from cloning ligation control. This colony contained a control insert that does not have a binding site for the aptamer primers PR1 and PR2 and therefore was not expected to produce any PCR products.

### 7.3.4 Sequencing of Cloned Vectors and Aptamer Analysis

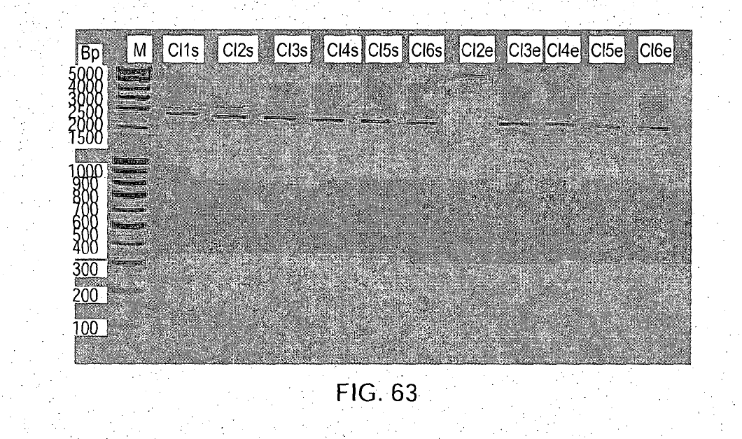

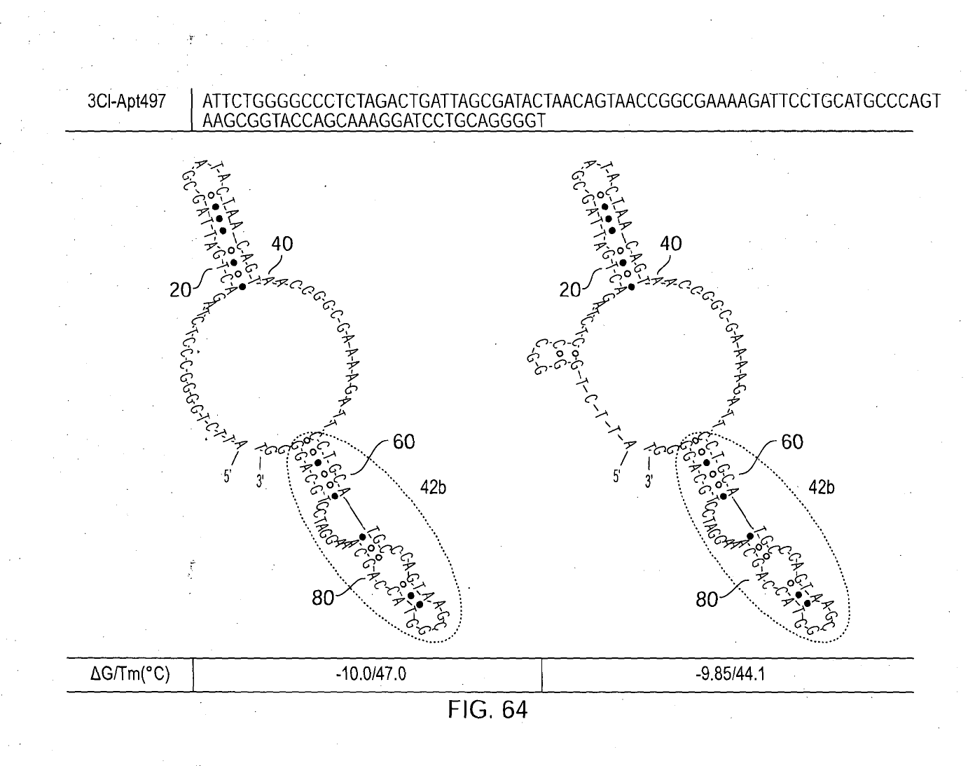

The cloned aptamer sequences against pathogenic E. coli O157 497 (CI2e-CI6e) and S. typhimurium 223 (CI1s-CI6s) were further analysed in order to obtain the DNA sequence. The positive colonies were transferred into a broth and incubated overnight. The plasmid DNA was purified from the overnight culture and the plasmid quality was tested by separating the samples on an agarose gel. In FIG. 63 the agarose gel image with the plasmid vectors is shown. It can be seen in the figure that all the other bands are the same size except the band in lane CI2e. This band is larger in size than the other bands and has also a faint band just above the dark band. Interestingly, this sample was the only white colony on the indicator plates after the second day of incubation while the other colonies were expressing the blue colour. The size of the samples (CI1s-CI6s and CI3e-CI6e) seems to be under 2000 bp when compared to the ladder in lane M. This is not the size that was expected since the plasmid is 3015 nt (+100 nt aptamer) long. The reason for this is the migration of the circular plasmid on the gel. It has been recognised that a circular plasmid migrates more rapidly on agarose gel than a linearised plasmid. Therefore the molecular weight marker used in this experiment is not suitable. Four samples for each strain (CI2e-CI5e and CI1 s-CI4s) were sequenced.

Four samples for both E. coli O157 (CI2e-CI5e) and S. typhimurium (CI1s-CI4s) were selected for sequencing. The plasmid DNA was sequenced using the sequencing primers T7 and Sp6r. The aptamer sequences were analysed using the aptamer primers PR1 and PR2 and by finding the matching sequences. The sequencing was successful for all of the samples that had a band in the level of 2000 bp compared to the molecular weight marker in FIG. 63. The sample CI2e that looked different on the gel and was the only sample expressing the blue colour on the second indicator plate failed the sequencing. No results were obtained from this sequencing reaction. It is possible the plasmid was damaged in this sample as two bands can be seen on the gel image (FIG. 63). Most likely the colony growing on the plate did contain the plasmid DNA that was used for cloning because the PCR reaction resulted in positive result. The nucleotide sequences of the successfully sequenced aptamers are presented in the Table below.

TABLE
Cloned aptamer sequences

| | Size/ | | |
|---|---|---|---|
| Sample | Bases | Primer | Sequence |
| 3Cl- | 100 | T7 | ATTCTGGGGCCCTCTAGACTGATTAGCGATACT [symbol] |
| Apt497 | | | [symbol] GGTACCAGCAAAGGATCCTGCAGGGGT (SEQ ID No. 11) |
| | 100 | Sp6r | ACCCCTGCAGGATCCTTTGCTGGTACC [symbol] |
| | | | [symbol] AGTATCGCTAATCAGTCTAGAGGGCCCCAGAAT (SEQ ID No. 12) |
| 4Cl- | 100 | T7 | ATTCTGGGGCCCTCTAGACTGATTAGCGATACT [symbol] |
| Apt497 | | | [symbol] GGTACCAGCAAAGGATCCTGCAGGGGT (SEQ ID No. 13) |
| | 100 | Sp6r | ACCCCTGCAGGATCCTTTGCTGGTACC [symbol] |
| | | | [symbol] AGTATCGCTAATCAGTCTAGAGGGCCCCAGAAT (SEQ ID No. 14) |
| 5Cl- | 100 | T7 | ATTCTGGGGCCCTCTAGACTGATTAGCGATACT [symbol] |
| Apt497 | | | [symbol] GGTACCAGCAAAGGATCCTGCAGGGGT (SEQ ID No. 15) |
| | 100 | Sp6r | ACCCCTGCAGGATCCTTTGCTGGTACC [symbol] |
| | | | [symbol] AGTATCGCTAATCAGTCTAGAGGGCCCCAGAAT (SEQ ID No. 16) |
| 1Cl- | 100 | T7 | ACCCCTGCAGGATCCTTTGCTGGTACC [symbol] |
| Apt223 | | | [symbol] AGTATCGCTAATCAGTCTAGATGGCCCCAGAAT (SEQ ID No. 17) |
| | 100 | Sp6r | ATTCTGGGGCCATCTAGACTGATTAGCGATACT [symbol] |
| | | | [symbol] GGTACCAGCAAAGGATCCTGCAGGGGT (SEQ ID No. 18) |
| 2Cl- | 100 | T7 | ACCCCTGCAGGATCCTTTGCTGGTACC [symbol] |
| Apt223 | | | [symbol] AGTATCGCTAATCAGTCTAGAGGGCCCCAGAAT (SEQ ID No. 19) |
| | 100 | Sp6r | ATTCTGGGGCCCTCTAGACTGATTAGCGATACT [symbol] |
| | | | [symbol] GGTACCAGCAAAGGATCCTGCAGGGGT (SEQ ID No. 20) |
| 3Cl- | 100 | T7 | ACCCCTGCAGGATCCTTTGCTGGTACC [symbol] |
| Apt223 | | | [symbol] AGTATCGCTAATCAGTCTAGAGGGCCCCAGTAT (SEQ ID No. 21) |
| | 100 | Sp6r | ATACTGGGGCCCTCTAGACTGATTAGCGATACT [symbol] |
| | | | [symbol] GGTACCAGCAAAGGATCCTGCAGGGGT (SEQ ID No. 22) |
| 4Cl- | 100 | T7 | ACCCCTGCAGGATCCTTTGCTGGTACC [symbol] |
| Apt223 | | | [symbol] AGTATCGCTAATCAGTCTAGAGGGCCCCAGAAT (SEQ ID No. 23) |
| | 100 | Sp6r | ATTCTGGGGCCCTCTAGACTGATTAGCGATACT [symbol] |
| | | | [symbol] GGTACCAGCAAAGGATCCTGCAGGGGT (SEQ ID No. 24) |

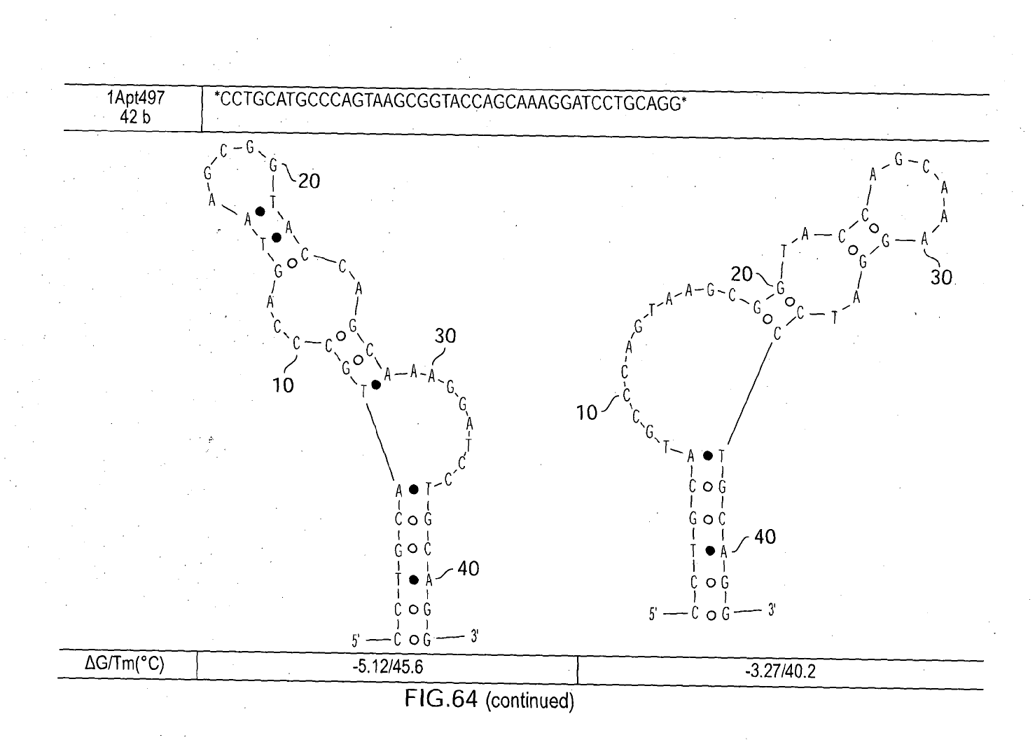

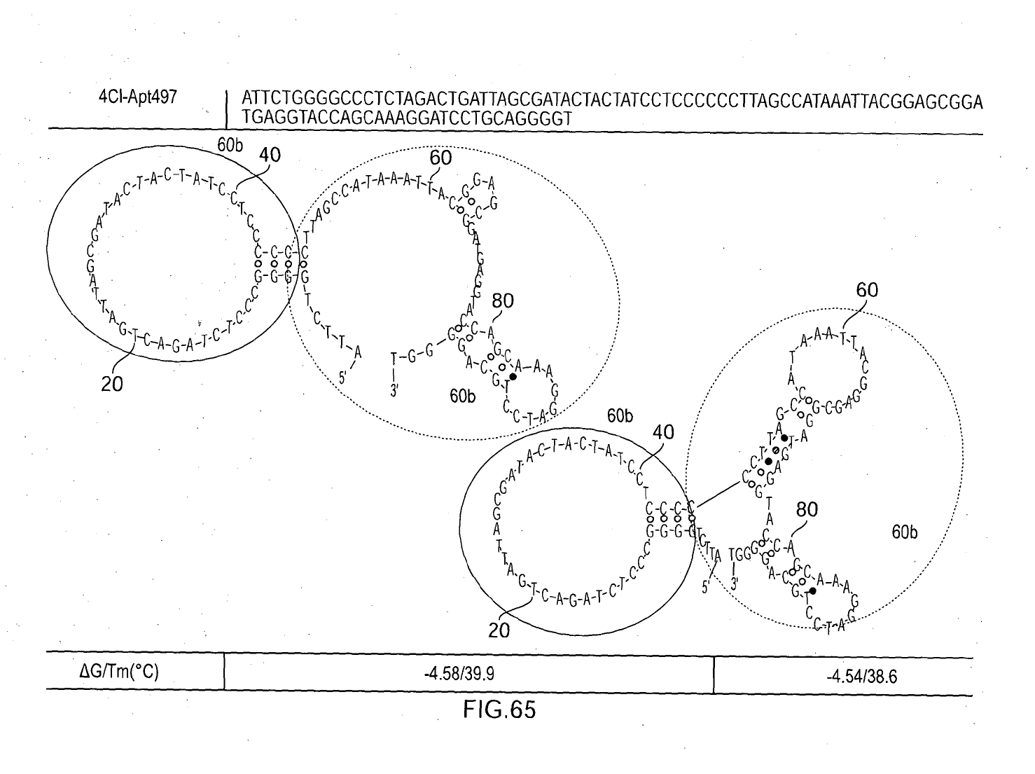

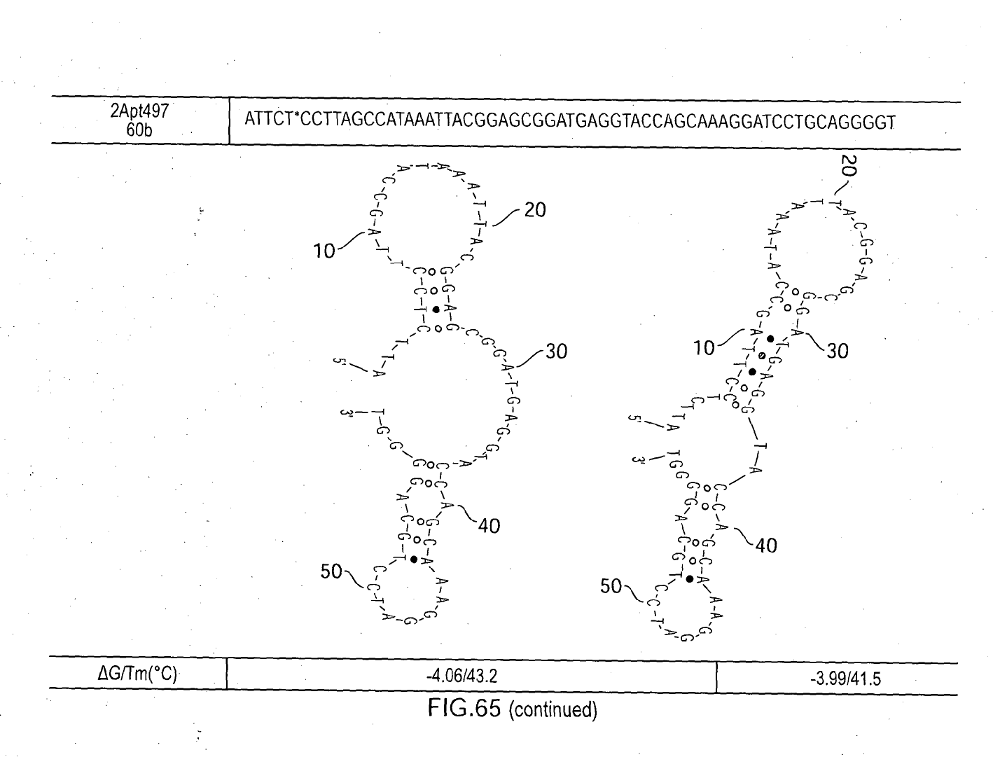

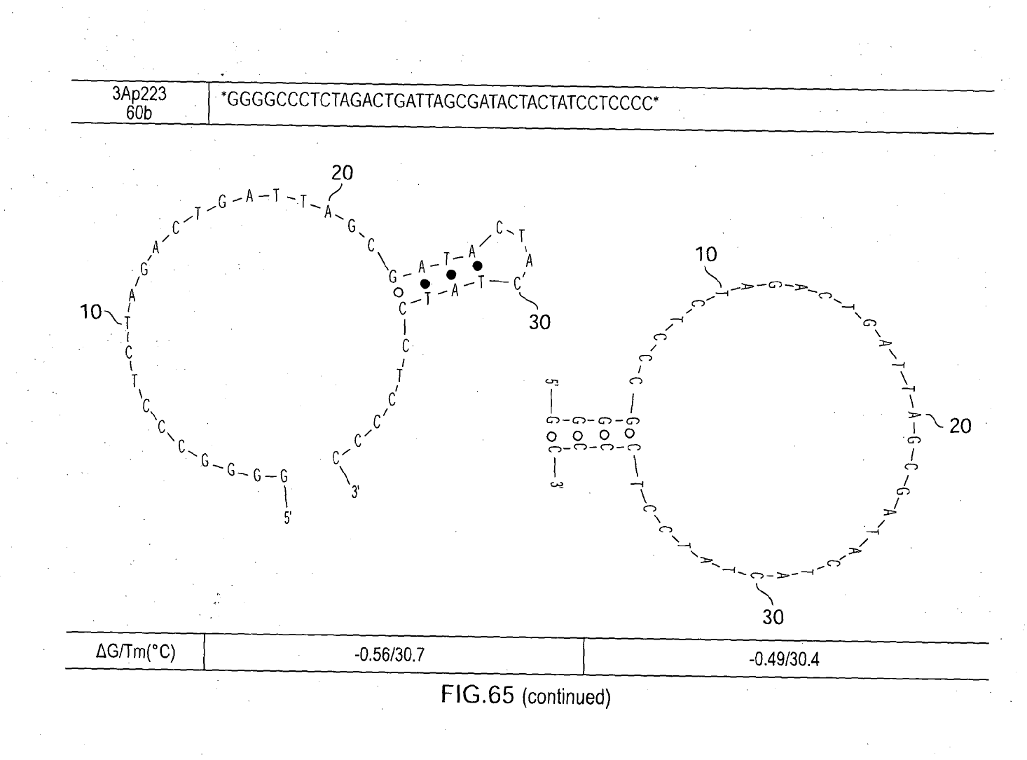

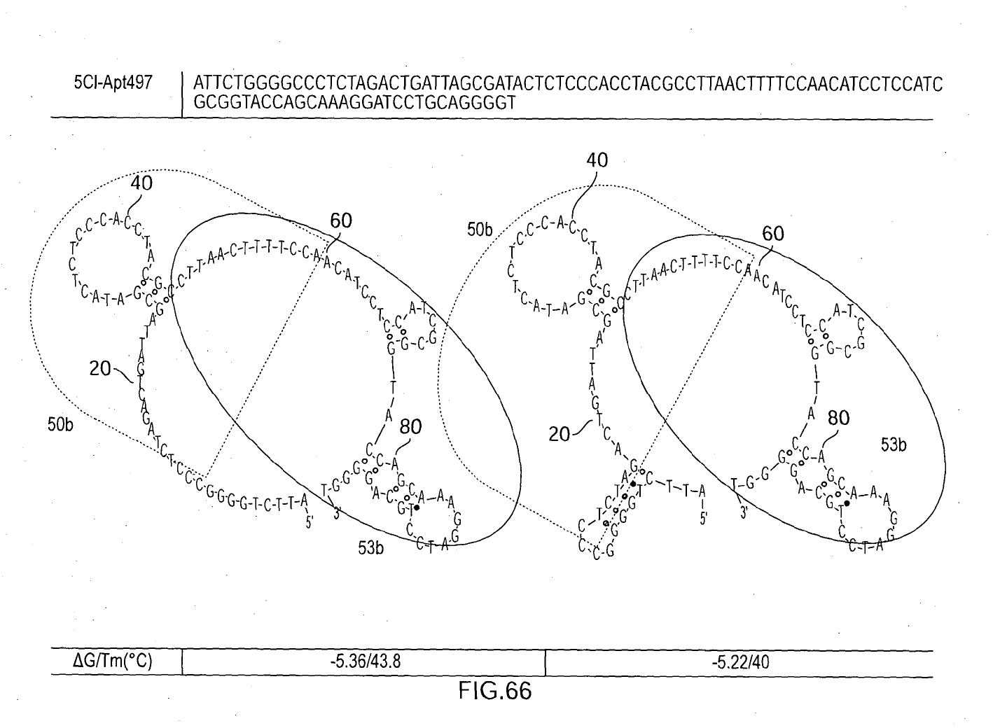

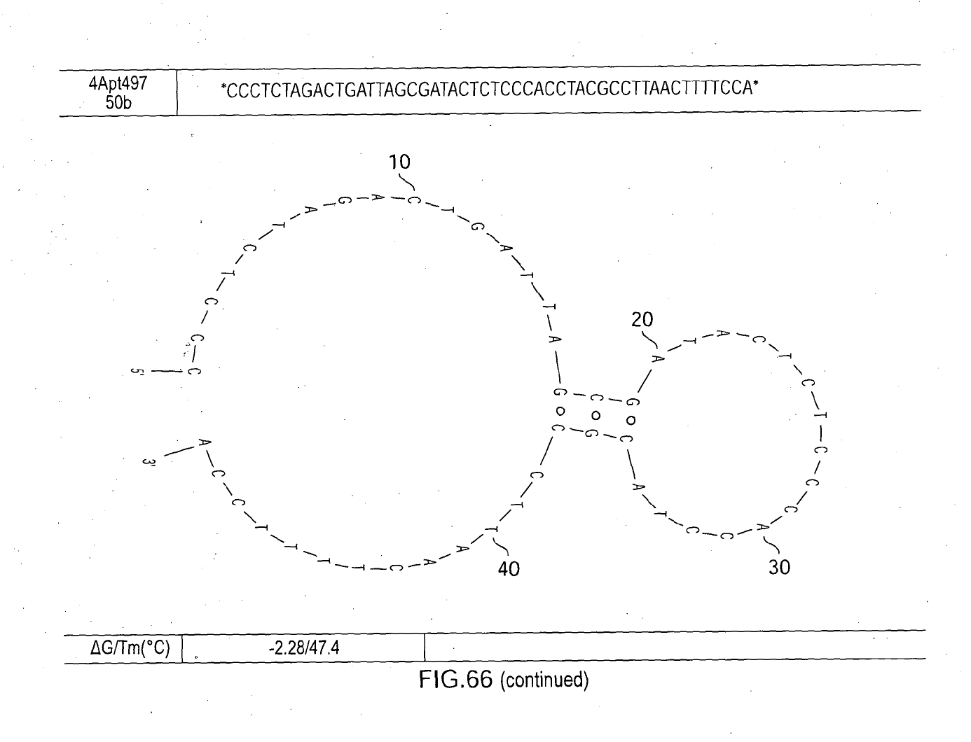

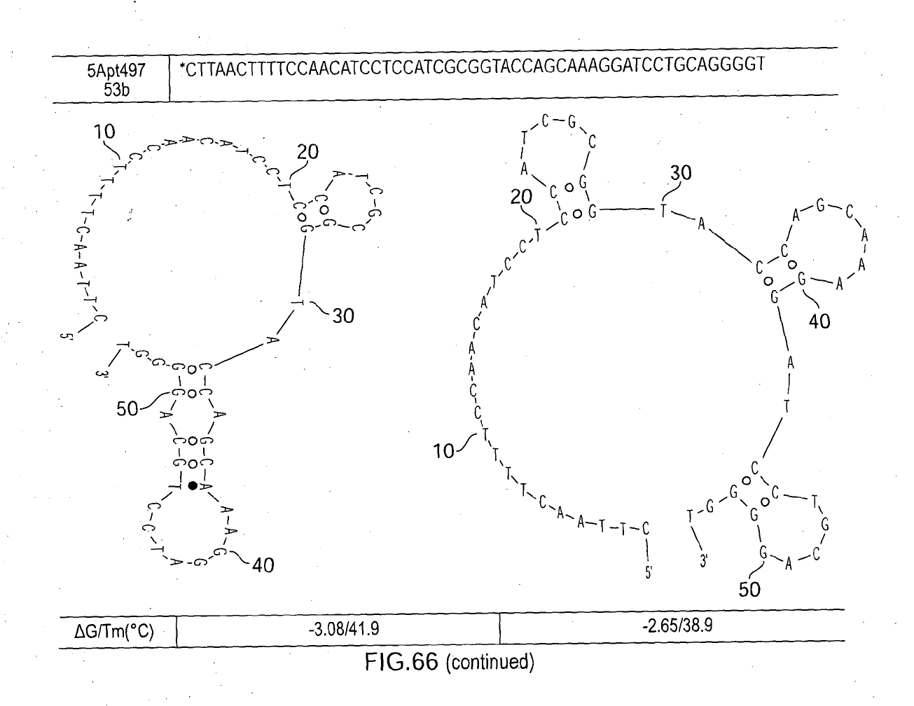

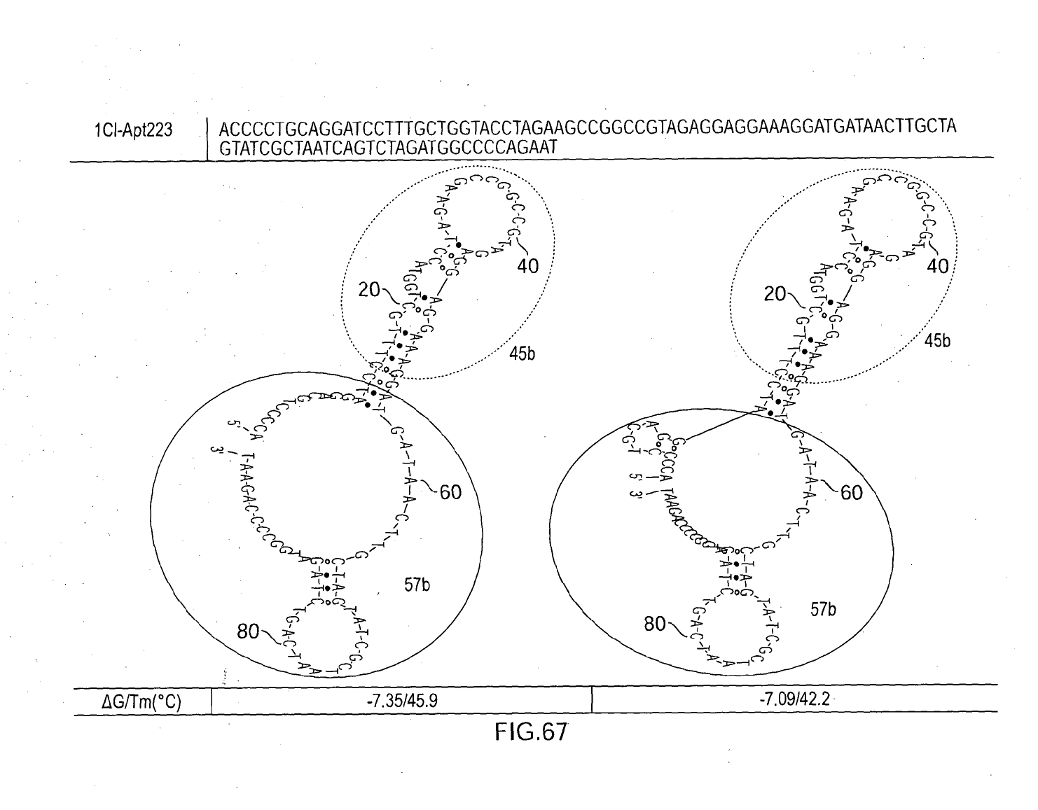

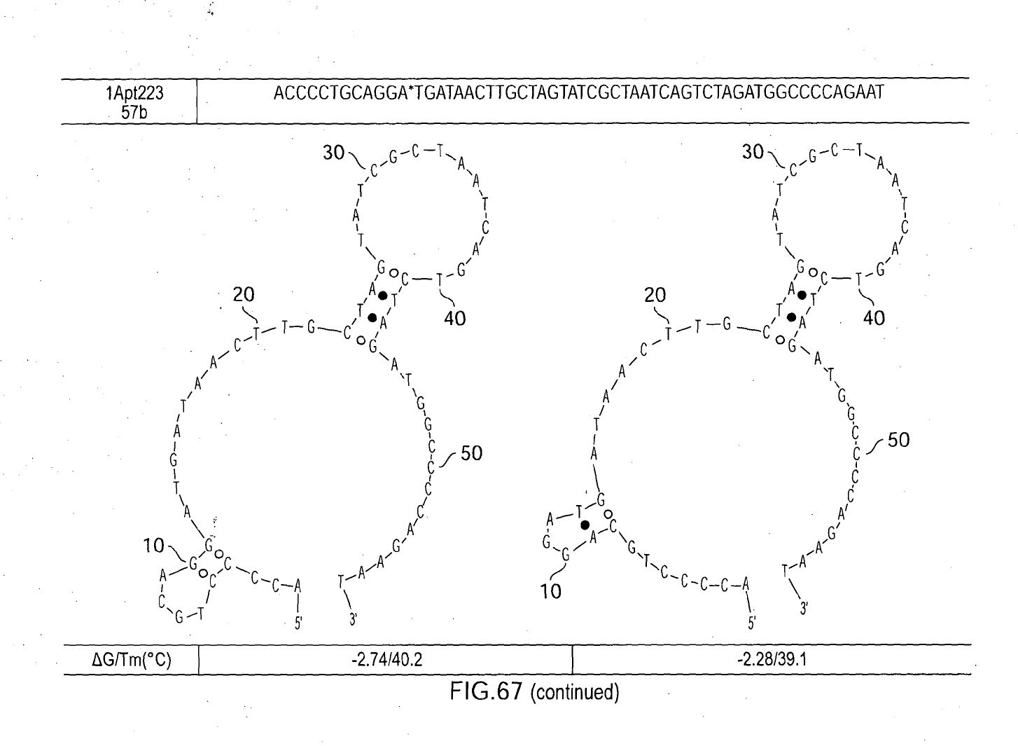

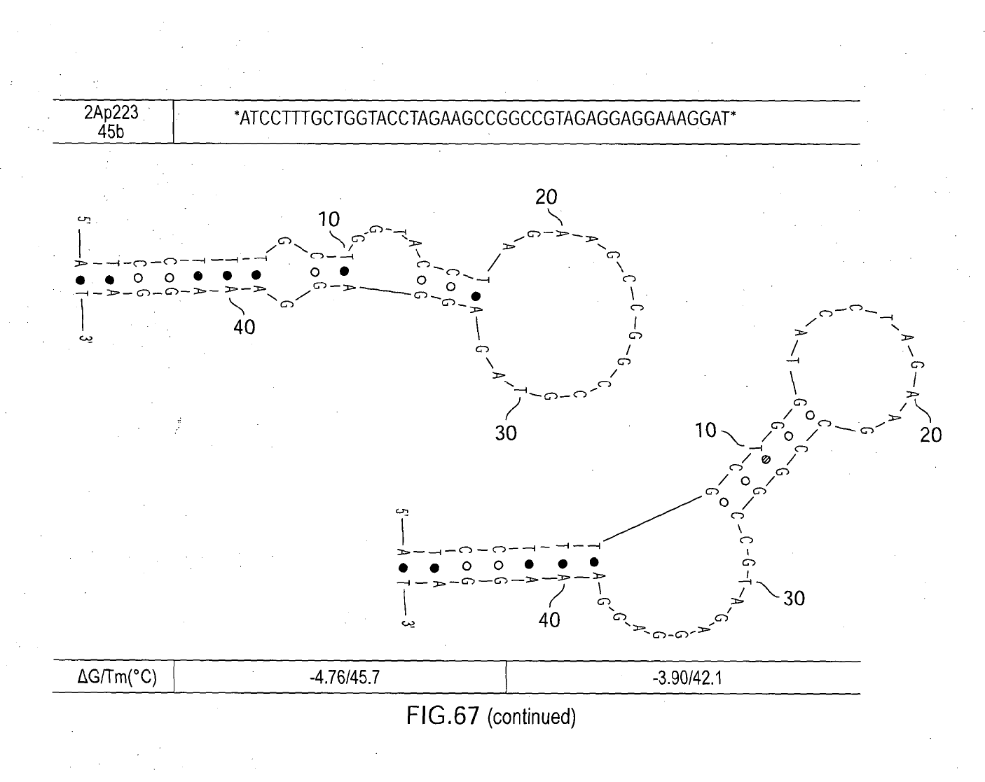

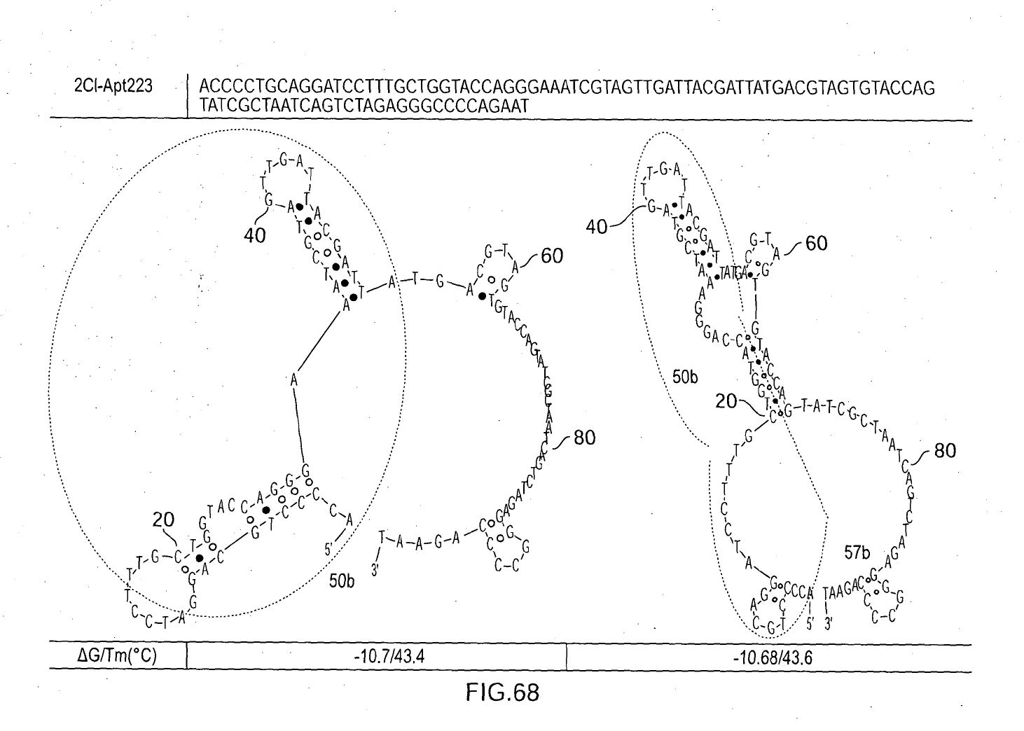

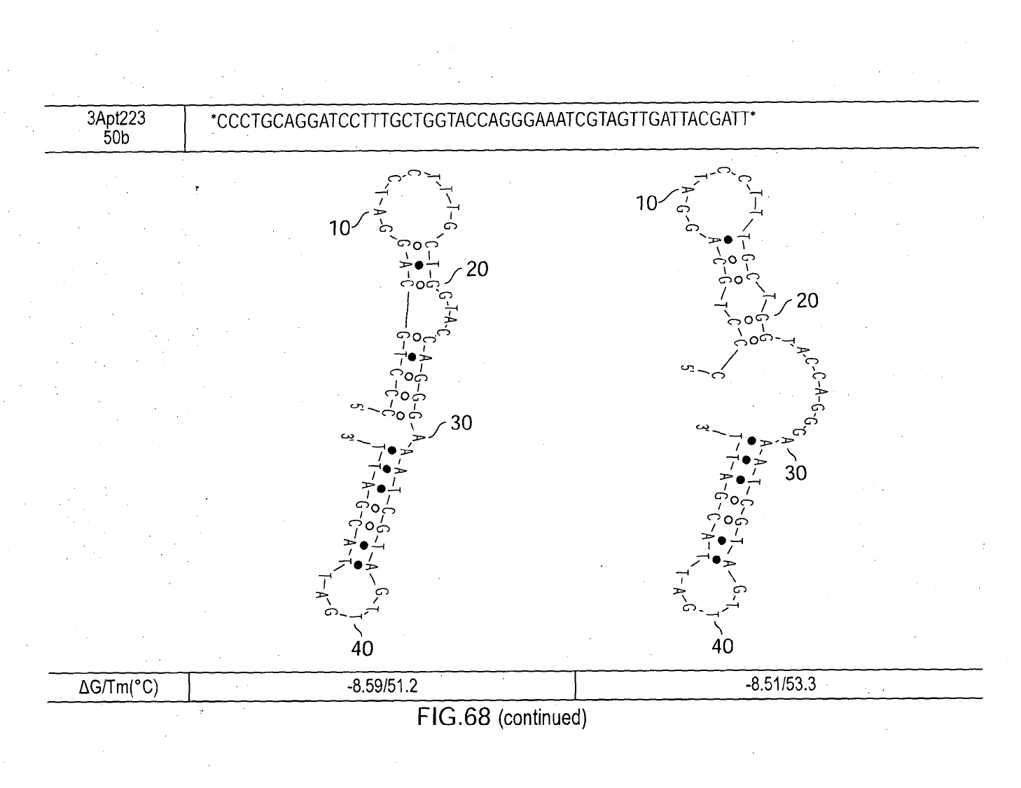

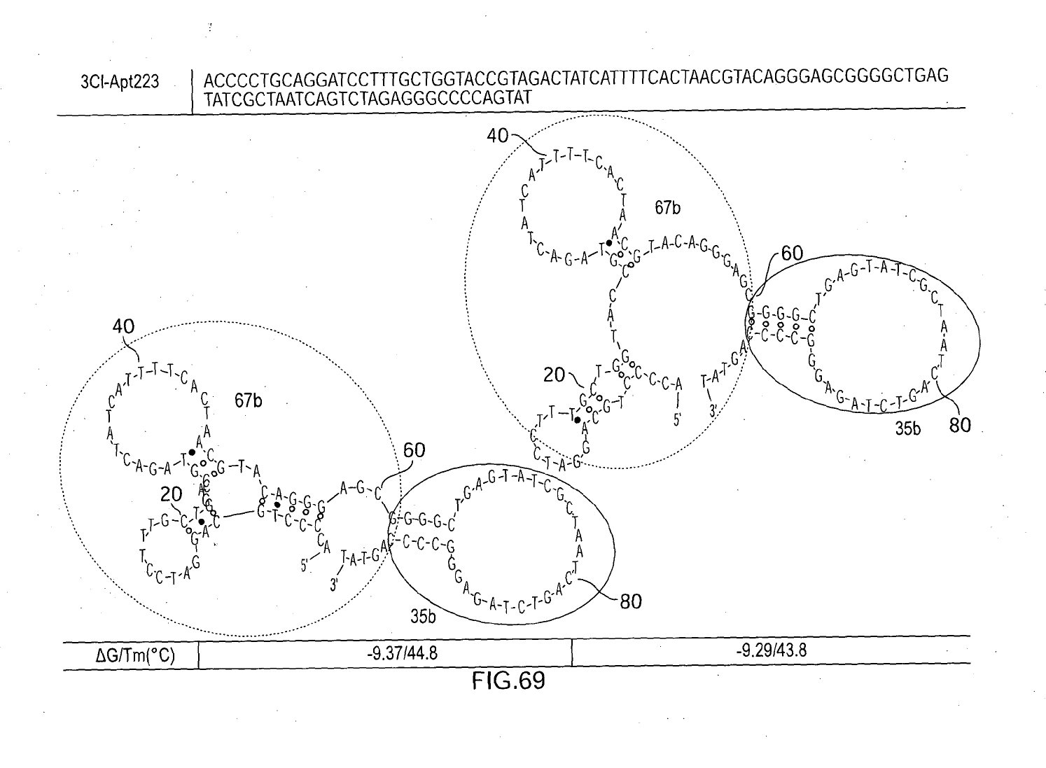

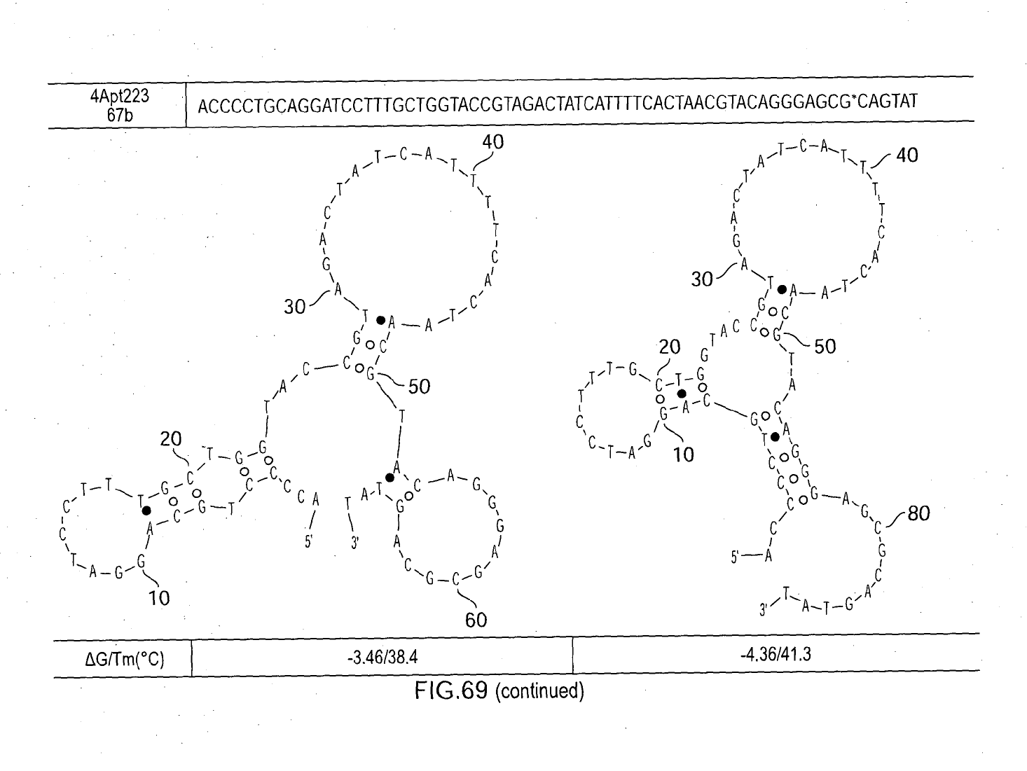

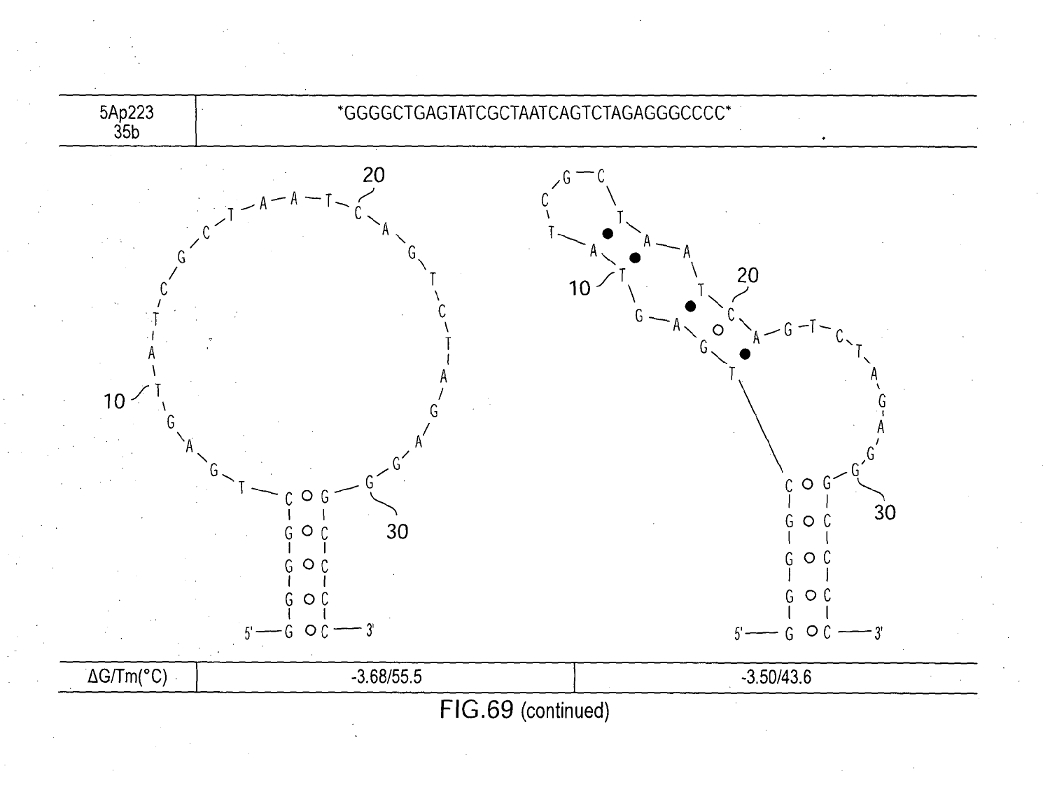

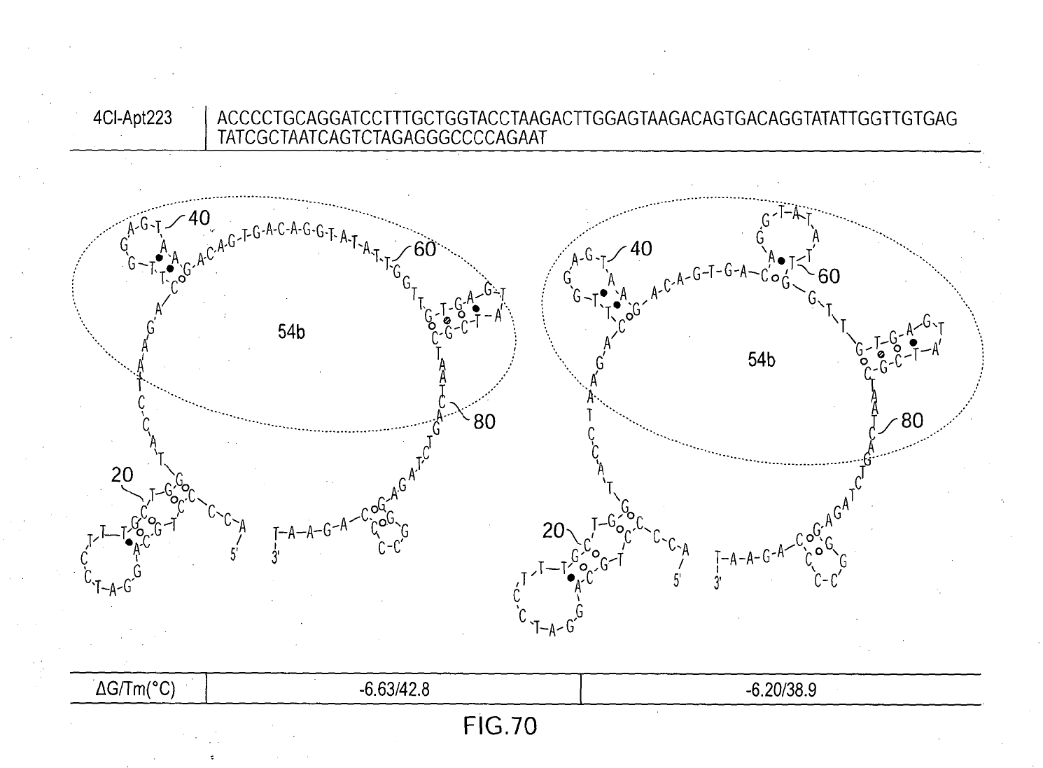

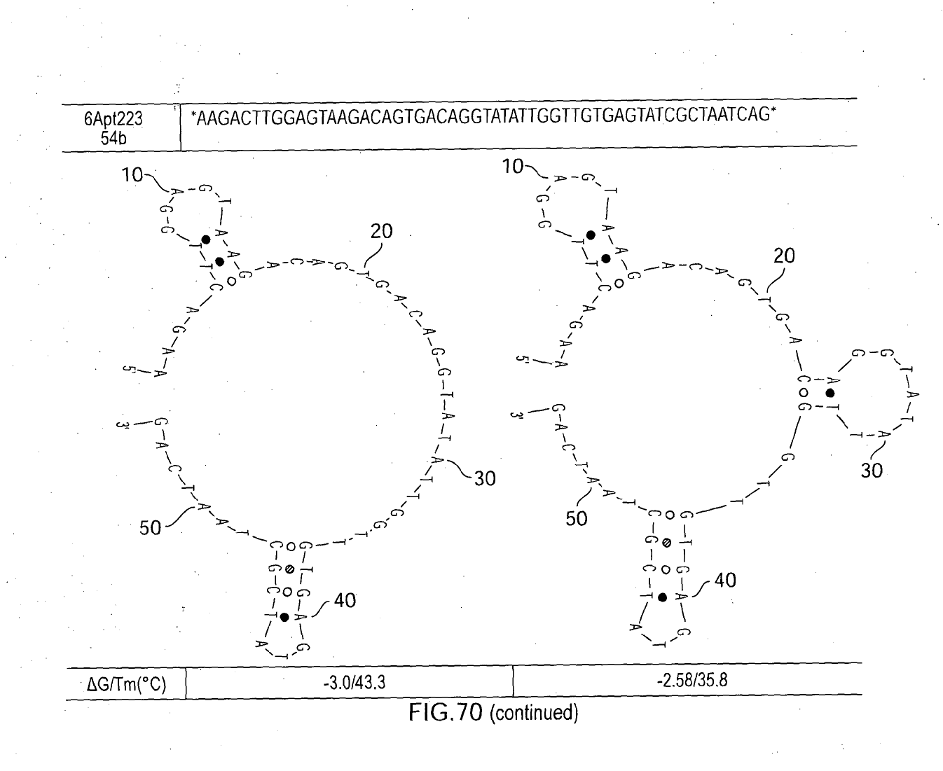

In order to synthesise the aptamers the number of nucleotides had to be reduced (James, 2000). The aptamer sequences (rows T7 in the Table above) 3CI-Apt497, 4CI-Apt497, 5CI-Apt497, 1 CI-Apt223, 2CI-Apt223, 3CI-Apt223 and 4CI-Apt223 were further analysed using the UNAFold program that gives aptamer secondary structures most likely to be formed in buffer conditions. The structures are presented in FIG. 64 (3CI-Apt497), FIG. 65 (4CI-Apt497), FIG. 66 (5CI-Apt497), FIG. 67 (1CI-Apt223), FIG. 68 (2CI-Apt223), FIG. 69 (3CI-Apt223) and FIG. 70 (4CI-Apt223). Two structures for 100 bp sequences are presented in the first line and the aptamer sequences that were cut off from these 100 bp sequences are presented below. The sequences with reduced lengths are marked by circle or circles in the pictures and the strongest isolated sequences with their secondary structures are shown for each sample below. For aptamer 5Apt497 (FIG. 66) only one structure is shown because this is the only way the sequence can fold according to UNAFold. The ΔG was calculated by the UNAFold and it is defined as a change in Gibbs energy when the system undergoes a thermodynamic change. Generally two different sequences were isolated from the 100 bp sequence but in some sequences only one sequence could have be isolated (3CI-Apt497, 2CI-Apt223, 4CI-Apt223).

The secondary structures as well as energy needed to break the structure (ΔG) were compared and the three strongest aptamers for each pathogenic strain (S. typhimurium and E. coli O157) were selected and synthesised with FAM-labels. The synthesised aptamers along with their size and ΔG values are listed in the Table below. The aptamer 4Apt497 was selected instead of aptamer 5Apt497 that has a ΔG value −3.08 because aptamer 4Apt497 has only a one secondary structure (FIG. 66). It would be interesting to see if this type of aptamer binds better than the aptamers that can form different type of secondary structures. These six aptamers were further analysed.

TABLE
Sequences and ΔG values for the
synthesised FAM-labelled aptamers

| Aptamer | Nt | Sequence | ΔG |
|---|---|---|---|
| 1Apt497 | 42 | 5′FAM-CCTGCATGCCCAGTAAGCGG | −5.12 |
| | | TACCAGCAAAGGATCCTGCAGG-3′ | |
| | | (SEQ ID No. 5) | |
| 2Apt497 | 60 | 5′FAM-ATTCTCCTTAGCCATAAATT | −4.06 |
| | | ACGGAGCGGATGAGGTACCAGCAAAG | |
| | | GATCCTGCAGGGGT-3′ | |
| | | (SEQ ID No. 6) | |
| 4Apt497 | 50 | 5′FAM-CCCTCTAGACTGATTAGCGA | −2.28 |
| | | TACTCTCCCACCTACGCCTTAACTTT | |
| | | TCCA-3′ (SEQ ID No. 7) | |
| 2Apt223 | 45 | 5′FAM-ATCCTTTGCTGGTACCTAGA | −4.76 |
| | | AGCCGGCCGTAGAGGAGGAAAGGAT-3′ | |
| | | (SEQ ID No. 8) | |
| 3Apt223 | 50 | 5′FAM-CCCTGCAGGATCCTTTGCTG | −8.59 |
| | | GTACCAGGGAAATCGTAGTTGATTAC | |
| | | GATT-3′ (SEQ ID No. 9) | |
| 5Apt223 | 35 | 5′FAM-GGGGCTGAGTATCGCTAATC | −3.68 |
| | | AGTCTAGAGGGCCCC-3′ | |
| | | (SEQ ID No. 10) | |

### 7.3.5 Binding of the Cloned Aptamers

7.3.5.1 E. coli O157 Aptamers

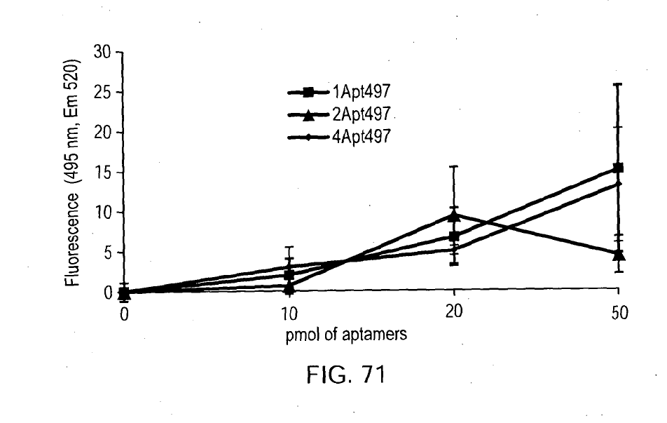

Aptamers selected to bind to pathogenic E. coli O157 497 were cloned and sequenced. The aptamer sequences (1Apt497, 2Apt497 and 4Apt497) were synthesised with a fluorescent label and the binding was tested against live E. coli O157 497 bacterial cells. The aptamers (10, 20 and 50 pmol) were incubated with the bacterial cells in triplicate. The binding was analysed with a fluorimetry analysis and the samples were examined under the fluorescence microscope. The fluorescence values are presented in FIG. 71. The result shows that when the aptamers have been added the fluorescence values are greater. Even though the error bars are large the data analysis showed that the fluorescence is significantly higher in all samples 1Apt497 (F=4.24, p=0.05), 2Apt497 (F=4.5, p=0.03) and 4Apt497 (F=6.47, p=0.02) when the aptamers have been added. According to the results an addition of 20 pmol of aptamers is enough to detect the fluorescence in the samples 1Apt497 (F=10, p=0.03) and 4Apt497 (F=112.5, p=4.5×10⁻⁴) since the addition of 50 pmol of aptamer 1Apt497 did not result in significantly higher fluorescence. For aptamer 2Apt497 the addition of 50 pmol is required (F=8.5, p=0.04). These results indicate the aptamers 1Apt497, 2Apt497 and 4Apt497 are binding to E. coli O157 497 bacterial cells. The large error bars could be caused by the variable number of the bacterial cells. It is possible that too many bacterial cells have been washed off during the washing steps.

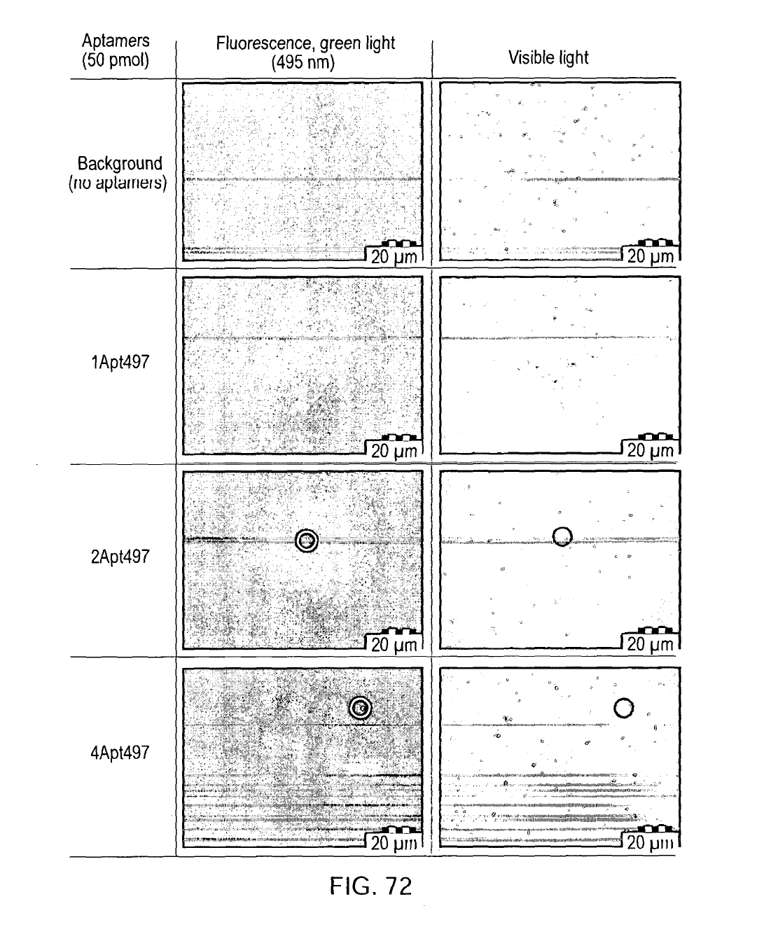

Samples were visualised under the microscope with a green fluorescence and a visible light. There was no great difference between the images taken of the samples with different aptamer concentrations. The images taken from 20 pmol samples are shown in FIG. 72 where the fluorescence microscope images are on the left hand side and the visible light images on the right hand side. The bacterial cells with fluorescent labelled aptamers bound to them can be seen on the images as bright green dots. No aptamers were added to the background samples. It can be seen in the images that not many bright dots are visible compared to the background. It was noticeable when looking at the microscope images that there were no bright dots all over the microscope slide. It can be possible that some aptamers have bound to the surface of the bacterial cells but they cannot be detected in the microscope images. The fluorescent FAM label that is attached to the aptamers is only a small molecule (6-Carboxyfluorescein). If only one aptamer has bound to a bacterial cell it is very likely that this bacterial cell is not visible in the microscope image. Although some bright dots that were not visible in background samples can be detected in the image where the aptamers were added. This result indicates that some aptamer binding can be detected. E. coli O157 bacterial cells are relatively small in size and move rapidly on the microscope slide. This made the visualisation of the bacterial cells challenging and therefore some of the images are not clear. Bright green bacterial cells were often seen when two bacterial cells were connected to each other (dividing cells). This can be seen for example in the fluorescence images of sample 2Apt497 and 4Apt497.

7.3.5.2 Binding of E. coli O157 Aptamers to E. coli K12

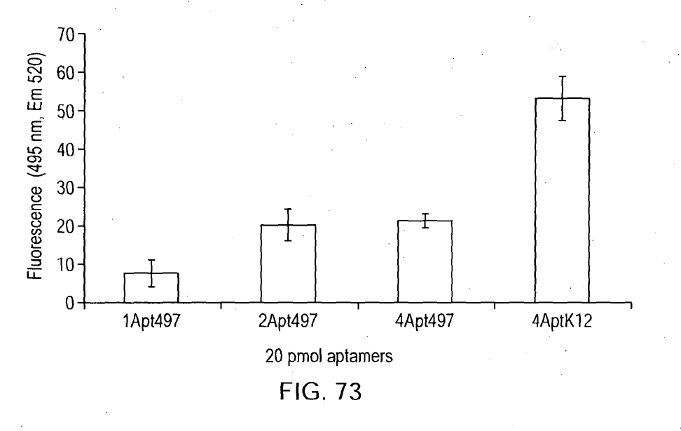

E. coli O157 aptamers were selected from a pool of E. coli K12 binding aptamers. The binding of these three cloned aptamers 1Apt497, 2Apt497 and 4Apt497 was tested against E. coli K12 by the fluorimetry analysis. The aptamers (20 pmol) were incubated with the bacterial cells and the fluorescence was measured. Aptamer 4AptK12 was used as a positive control as the binding of this aptamer has previously been demonstrated (5.3.4.1). The fluorescence values that are presented in FIG. 73 were measured for all the samples and some binding can be detected from each sample. The fluorescence values for all four samples are significantly higher when the aptamers have been added compared to the background fluorescence (1Apt497, F=7.56 p=0.05; 2Apt497 F=39.8, p=8.1×10⁻³; 4Apt497 F=105.6, p=5.0×10⁻⁴; 4AptK12 F=195.9, p=1.5×10⁻⁴). It can be seen in the figure that the fluorescence was significantly higher when the aptamer 4AptK12 was incubated with E. coli K12 bacterial cells than when the E. coli O157 aptamers 1Apt497 (F=136.1 p=3.1×10⁻⁴), 2Apt497 (F=46.2 p=6.5×10⁻³) and 4Apt497 (F=84.0 p=7.9×10⁻⁴) were incubated. This result indicates the aptamers selected to bind to E. coli O157 497 from a pool of E. coli K12 aptamers are also binding to E. coli K12 but not as strongly as the E. coli K12 binding aptamers. Due to a problem with the fluorescence plate reader the fluorescence of the samples was measured with a sensitivity of 75 instead of 50. This means that the fluorescence values cannot be compared directly to the values obtained before. When looking at the fluorescence values previously measured for E. coli K12 aptamer 4AptK12, for example in FIG. 47, it can be seen that the fluorescence is about 30 units less than the measurement (FIG. 73) with the sensitivity of 75. This information can be used when comparing the results and in that case the fluorescence measured for the aptamers 1Apt497, 2Apt497 and 4Apt497 are showing more binding against E. coli O157 497 (FIG. 71) than against E. coli K12 (FIG. 73).

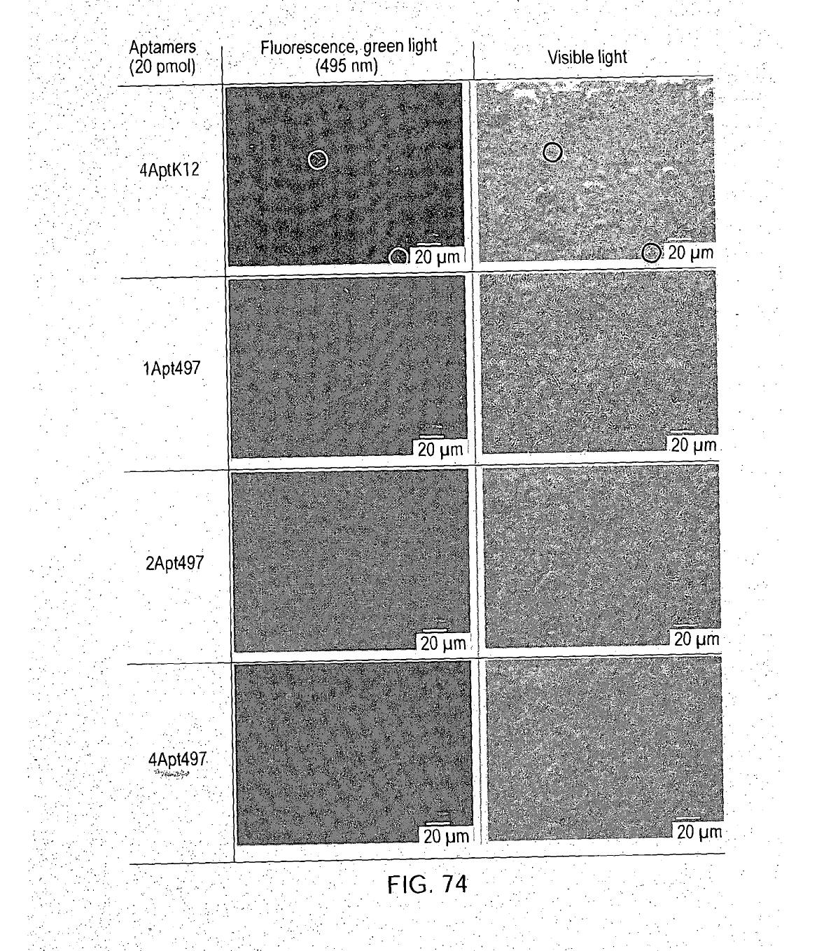

The samples were visualised under the fluorescence microscope with 60× magnification. The microscope images are presented in FIG. 74. The fluorescence images are on left hand side and the visible light images on right hand side. It can be seen in the images that when the E. coli K12 bacteria cells were incubated with the aptamer 4AptK12 (positive control), bright green dots can be seen. Some fluorescent dots can also be seen when E. coli K12 was incubated with the pathogen aptamers 1Apt497, 2Apt497 and 4Apt497 but the fluorescence is not as bright in the aptamer 4AptK12 image. This result confirms some binding of the aptamer 1Apt497, 2Apt497 and 4Apt497 to E. coli K12 can be detected.

7.3.5.3 S. typhimurium Binding Aptamers

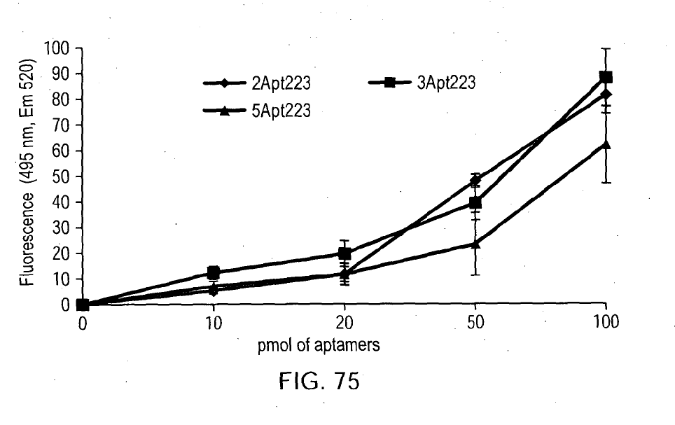

The aptamers that were selected to bind to S. typhimurium 223 were cloned and the sequences were synthesised with fluorescent labels. The binding of the FAM-aptamer sequences 2Apt223, 3Apt223 and 5Apt223 was tested against live S. typhimurium 223 bacterial cells. The aptamers (10, 20 and 50 pmol) were incubated with the bacterial cells in triplicate. The binding was analysed with the fluorimetry analysis. The fluorescence values are presented in FIG. 75. The results show that the greater the number of added aptamers the higher the fluorescence. The fluorescence is significantly higher in all samples 2Apt223 (F=233.8, p=7.9×10⁻¹⁰), 3Apt223 (F=87.8, p=9.54×10⁻⁸) and 5Apt223 (F=23.1, p=4.9×10⁻⁵) when the aptamer have been added. The results indicate that the addition of 10 pmol of S. typhimurium aptamers is enough to see a significantly higher fluorescence values than when the aptamers have not been added (2Apt223: F=64, p=1.3×10⁻³; 3Apt223: F=48.9, p=2.2×10⁻³; 5Apt223: F=25.9, p=7.0×10⁻³).

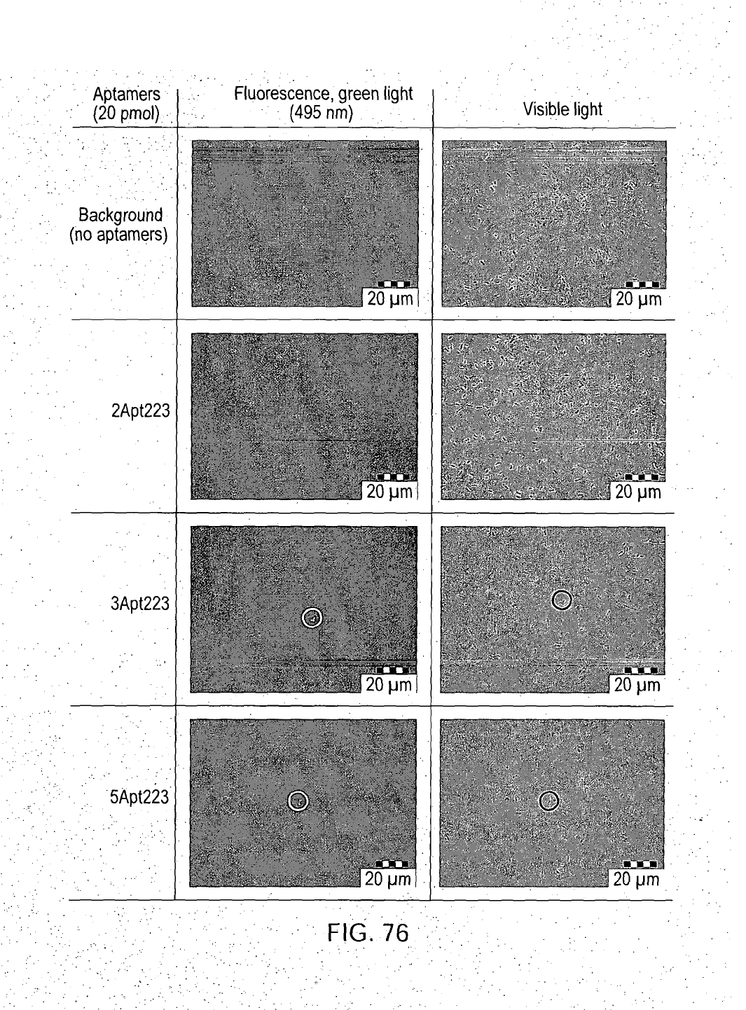

The samples were visualised under a microscope with a green fluorescent light and a visible light. There was no great difference between the images taken of the samples with different aptamer concentrations. The images for 20 pmol are shown in FIG. 76. The fluorescence microscope images are on the left hand side and the visible light images are on the right hand side. The bacterial cells with fluorescent labelled aptamers bound to them can be seen on the images as bright green dots. No aptamers were added to the background samples. In the images not many bright green bacterial cells can be seen. It is possible that the aptamers are only binding to some of the bacterial cell or only a small number of aptamers is binding to cell and therefore the bacterial cells cannot be detected in the images.

7.3.5.4 Specificity of the S. typhimurium Binding Aptamers

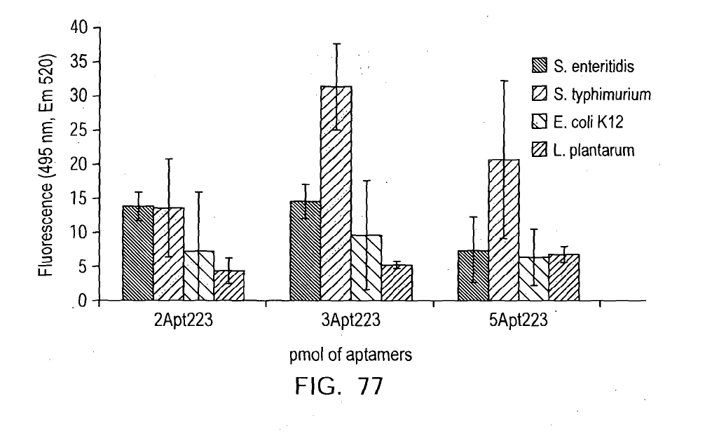

The specificity of the S. typhimurium aptamers 2Apt223, 3Apt223 and 5Apt223 was tested. The aptamers (20 pmol) were incubated with E. coli K12, L. plantarum and S. enteritidis and S. typhimurium. The binding was then analysed by the fluorimetry analysis and the fluorescence values are shown in FIG. 77. It can be seen in the figure that the fluorescence is significantly higher in samples 3AptK12 (F=17.3, p=2.3×10⁻³) and 5AptK12 (F=4.59, p=0.04) when incubated with S. typhimurium compared to when they were incubated with E coil K12, L. plantarum and S. enteritidis. The fluorescence of the aptamer 2Apt223 is not significantly higher when incubated with S. typhimurium (F=2.93, p=0.1) compared to the values from E coli K12, L. plantarum and S. enteritidis samples. The lower detection for this aptamer was also seen in FIG. 75. The aptamer 3Apt223 seem to be the strongest of these three aptamers according to the fluorescence values. This was expected because similar result was seen in the aptamer binding study in FIG. 75 when 20 pmol aptamers was used. These results indicate the aptamer 3Apt223 is having the strongest affinity and specificity of these three aptamers against S. typhimurium. It is possible that more specific aptamers could have been obtained by performing the counter selection.

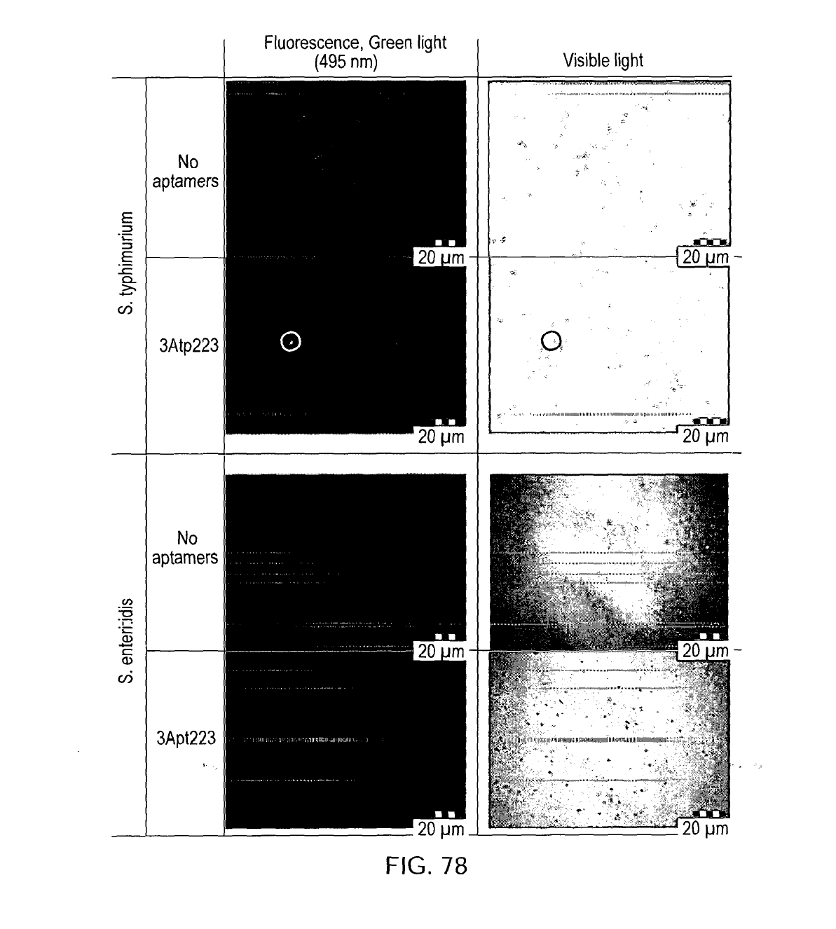

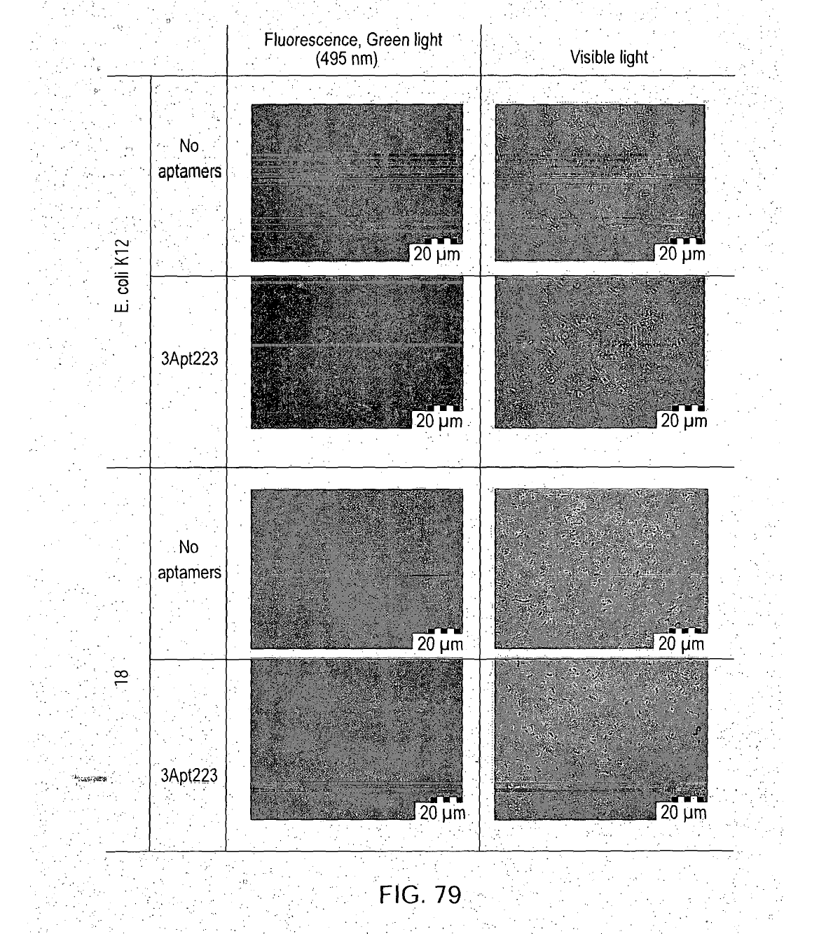

The samples were visualised under the microscope. As an example the microscope images for S. typhimurium, S. enteritidis, E. coli K12 and L. plantarum samples with 3Apt223 aptamers are presented in FIG. 78 and FIG. 79. The visible light images are presented on the left hand side and the fluorescence images on the right hand side. Samples 2Apt223 and 5Apt223 are not presented here because there is no great difference between the samples. A bright spot can be seen in the S. typhimurium fluorescence image (left hand side FIG. 78) when the aptamers have been added. In S. enteritidis and L. plantarum fluorescence images (left hand side, FIG. 78 and FIG. 79) no such a bright spots can be detected when the aptamers were added. In E. coli K12 sample (FIG. 79) the background control has some brighter spots. It was noticed that when this bacterium was looked at under the 100× magnification the background samples express some self-fluorescence. These green dots are most likely the nucleus inside the bacterial cells. The self-fluorescence was not visible when the bacterial cells were looked at under the 60× magnification (FIG. 32). In the E. coli K12 background sample bright green dots can be detected. When the aptamers were added, the background fluorescence can be seen in the images but also some brighter spots. This results show that some binding of the S. typhimurium aptamers can be detected against E. coli K12.

### 7.4 Conclusions

Aptamers were selected to bind to pathogenic E. coli 496, E. coli O157 497, L. innocua 17, L. monocytogenes 489, L. monocytogenes 490, S. enteritidis 1152 and S. typhimurium 223. The binding of the aptamer pools was tested against their targets. The ninth aptamer pools against E. coli O157 497 and S. typhimurium 223 were selected for cloning and sequencing. The aptamer structures were then analysed and three sequences for each strain were synthesised with fluorescent labels.

According to more sensitive PCR based detection (amplification of the aptamer pools) the binding was detected when the samples were separated on the agarose gel. Two aptamer pools against pathogens were then selected for cloning and sequencing. The binding of these aptamers was then tested against their targets. The three synthesised sequences against pathogenic E. coli O157 were showing some binding and the aptamers against S. typhimurium showed a good binding at the fluorimetry analysis. Some differences in binding were detected between different aptamers. The binding of the E. coli O157 aptamers, that were selected from a pool of E. coli K12 aptamers, was tested against E. coli K12 bacterial cells. The results showed these aptamers can also bind to E. coli K12 but the binding is not as strong as the binding of the E. coli K12 specific aptamers. The specificity test that was performed to S. typhimurium aptamers showed that some of the aptamers were capable of detecting other bacterial cells and only some of the aptamers were specific to S. typhimurium. This study showed that the aptamers can be selected to bind to food-borne pathogens.

Overall the inventors have shown that aptamers can be easily selected to specifically bind to live bacterial cells. Unlike the usual aptamer selection process where aptamers are selected to bind to extracted surface molecules, the inventors show herein that aptamers are successfully selected to bind to whole bacterial cells. Most importantly the inventors have also shown that these aptamers can be used in the detection of pathogens (e.g. food-borne pathogens) in complex matrixes, e.g. in contaminated food.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Bonwick, G. A. & Smith, C. J. (2004). Immunoassays: their history, development and current place in food science and technology. International Journal of Food Science and Technology, 39, 817-827.
Cao, X., Li, S., Chen, L., Ding, H., Xu, H., Huang, Y. Li, J., Liu, N., Cao W., Zhu, Y., Shen, B. & Shao, N. (2009). Combining use of a panel of ssDNA aptamers in the detection of Staphylococcus aureus. Nucleic Acids Research, 37, 4621-4628.
Ellington, A. D. & Szostak J. W. (1990). In vitro selection of RNA molecules that bind specific ligands. Nature, 346, 818-822.
Hamula, C. L. A., Zhang, H., Guan, L. L., Li, X-F. & Le, X. C. (2008). Selection of aptamers against live bacterial cells. Analytical Chemistry, 80, 7812-7819.
Iqbal, S. S., Mayo, M. W., Bruno, J. G., Bronk, B. V., Batt, C. A. & Chambers, J. P. (2000). A review of molecular recognition technologies for detection of biological threat agents. Biosensors & Bioelectronics, 15, 549-578.
Joshi, R., Janagama, H., Dwivedi, H. P., Kumar T. M. A. S., Jaykus, L-A, Schefers, J. & Sreevatsan, S. (2009). Selection, characterization, and application of DNA aptamers for the capture and detection of Salmonella enterica serovars. Molecular and Cellular Probes, 23, 20-28.
Karkkainen, R., Drasbek, M. R., McDowell, I., Smith, C. J., Young, N. W. G., and Bonwick, G. (2011) “Aptamers for safety and quality assurance in the food industry: detection of pathogens” International Journal of Food Science and Technology—In press DOI 10.1111/j.1365-2621.2010.02470.x
Karoonuthaisiri, N., Charlermroj, R., Uawisetwathana, U., Luxananil, P., Kirtikara, K. & Gajanandana, 0. (2009). Development of antibody array for simultaneous detection of foodborne pathogens. Biosensors & Bioelectronics, 24, 1641-1648.
Pendergrast, P. S., Marsh, H. N., Grate, D., Healy J. M. & Stanton, M. (2005). Nucleic acid aptamers for target validation and therapeutic applications. Journal of Biomolecular Techniques, 16, 224-234.
Symensma, T. L., Giver, L., Zapp, M., Takle, G. B. & Ellington, A. D. (1996). RNA aptamers selected to bind Human Immunodeficiency Virus Type 1 Rev in vitro are Rev responsive in vivo. Journal of Virology, 70, 179-187.
Takemura, K., Wang, P., Vorberg, I., Surewicz, W., Priola, S. A., Kanthasamy, A., Pottathil, R., Chen, S. G. & Sreevatsan, S. (2006). DNA aptamers that bind to PrP^{C} and not PrP^{Sc} show sequence and structure specificity. Experimental Biology and Medicine, 231, 204-214.
Tombelli, S., Minunni, M. & Mascini, M. (2007). Aptamers-based assays for diagnostics, environmental and food analysis. Biomolecular Engineering, 24, 191-200.
Tuerk, C. & Gold, L. (1990). Systematic evolution of ligands by exponential enrichment: RNA ligands to bacteriophage T4 DNA polymerase. Science, 249, 505-510.
Vivekananda, J. & Kiel, J. L. (2003). Methods and compositions for aptamers against anthrax. U.S. Pat. No. 6,569,630 B1.
Vivekananda, J. & Kiel, J. L. (2006). Anti-Francisella tularensis DNA aptamers detect tularemia antigen from different subspecies by Aptamer-linked immobilised Sorbent assay. Laboratory Investigation, 86, 610-618

1. A nucleic acid aptamer which is specifically binds a pathogenic microorganism.

2. A nucleic acid aptamer comprising the nucleotide sequence shown herein as SEQ ID No. 5, 6, 7, 8, 9, 10, 1, 2, 3 or 4 or a fragment thereof or a sequence which is at least 80% identical therewith, or a sequence which hybridises under stringent conditions therewith.

3. A nucleic acid aptamer according to claim 1 wherein the nucleotide sequence comprises the nucleotide sequence shown herein as SEQ ID No. 5, 6, 7, 8, 9 or 10 or a fragment thereof.

4. A nucleic acid aptamer according to claim 1 wherein the fragment is at least 20.

5. A nucleic acid aptamer according to claim 1 wherein the fragment is at most 70 nucleotides in length.

6. A nucleic acid aptamer according to claim 1 wherein the aptamer is in the region of 40 to 60 nucleotides in length.

7. A nucleic acid aptamer according to claim 1 wherein the nucleic acid has specificity against a live pathogenic bacterium.

8. A nucleic acid aptamer according to claim 1 wherein the nucleic acid is synthetic.

9. A nucleic acid aptamer according to claim 1 wherein the aptamer comprises a fluorescent label.

10. A kit comprising at least one nucleic acid aptamer according to claim 1 together with instructions on how to use the at least one nucleic acid aptamer.

11. A kit according to claim 10 wherein the kit comprises i) a biotin labelled aptamer and ii) an enzyme labelled streptavidin which enzyme reacts to provide a detectable label.

12. A kit according to claim 11 wherein the enzyme is peroxidase and the kit optionally comprises iii) a 2,2′-azino-bis(3-ethylbenzthiazoline-6-sulphonic acid (ABTS) substrate and iv) hydrogen peroxide.

13. A kit according to claim 10 wherein the kit comprises more than one nucleic acid aptamers.

14. A device comprising at least one of the nucleic aptamers according to claim 1.

15. A method of detecting a microorganism in a sample comprising admixing a nucleic acid aptamer according to claim 1 with the sample and identifying the presence of a bound aptamer.

16. A method according to claim 15 wherein the microorganism is a bacterium.

17. (canceled)

18. A method according to claim 15 wherein the nucleic acid aptamer has specificity against pathogenic microorganism.

19. A method according to claim 18 wherein the pathogenic microorganism is selected from the group consisting of Salmonella spp., Escherichia coli spp., and Listeria spp.

20. (canceled)

21. A method according to claim 15 wherein the sample is a food or feed sample, a beverage, a pharmaceutical sample, a personal care sample, a raw ingredient, a finished product or is taken from the environment of manufacture or storage.

22-32. (canceled)

33. A method of selecting aptamers, wherein the aptamer is selected on its ability to bind to live bacterial cells, which method comprises the steps of exposing an aptamer to live bacterial cells and selecting an aptamer which binds to said live bacterial cells, optionally said method further comprises a washing and centrifuging step.

34. The method of claim 33 wherein the method comprises two washing and centrifuging steps.

35. The method according to claim 34, wherein the first washing and centrifuging step occurs before aptamer binding and the second washing and centrifuging step occurs after aptamer binding.

36. (canceled)


SEQUENCE LISTING (53 sequences)

SEQ ID NO: 1 - DNA, 54 bp, Artificial Sequence
  Description of Artificial Sequence Synthetic oligonucleotide
acccctgcaggatcctttgctggtaccccgcgcgttatttccctgccccagaat

SEQ ID NO: 2 - DNA, 51 bp, Artificial Sequence
  Description of Artificial Sequence Synthetic oligonucleotide
ccctccctcatccgttgtctcgctcagagtatcgctaatcagtctagaggg

SEQ ID NO: 3 - DNA, 54 bp, Artificial Sequence
  Description of Artificial Sequence Synthetic oligonucleotide
attctggggccctctagactgattagcgatactacttaacctgcatgcaggggt

SEQ ID NO: 4 - DNA, 64 bp, Artificial Sequence
  Description of Artificial Sequence Synthetic oligonucleotide
acccctgcaggatcctttgctggtaccgcgttatgggaaaatcaggagagaggggccccagaat

SEQ ID NO: 5 - DNA, 42 bp, Artificial Sequence
  Description of Artificial Sequence Synthetic oligonucleotide
cctgcatgcccagtaagcggtaccagcaaaggatcctgcagg

SEQ ID NO: 6 - DNA, 60 bp, Artificial Sequence
  Description of Artificial Sequence Synthetic oligonucleotide
attctccttagccataaattacggagcggatgaggtaccagcaaaggatcctgcaggggt

SEQ ID NO: 7 - DNA, 50 bp, Artificial Sequence
  Description of Artificial Sequence Synthetic oligonucleotide
ccctctagactgattagcgatactctcccacctacgccttaacttttcca

SEQ ID NO: 8 - DNA, 45 bp, Artificial Sequence
  Description of Artificial Sequence Synthetic oligonucleotide
atcctttgctggtacctagaagccggccgtagaggaggaaaggat

SEQ ID NO: 9 - DNA, 50 bp, Artificial Sequence
  Description of Artificial Sequence Synthetic oligonucleotide
ccctgcaggatcctttgctggtaccagggaaatcgtagttgattacgatt

SEQ ID NO: 10 - DNA, 35 bp, Artificial Sequence
  Description of Artificial Sequence Synthetic oligonucleotide
ggggctgagtatcgctaatcagtctagagggcccc

SEQ ID NO: 11 - DNA, 100 bp, Artificial Sequence
  Description of Artificial Sequence Synthetic polynucleotide
attctggggccctctagactgattagcgatactaacagtaaccggcgaaaagattcctgcatgcccagtaagcggtaccagcaaaggatcctgcaggggt

SEQ ID NO: 12 - DNA, 100 bp, Artificial Sequence
  Description of Artificial Sequence Synthetic polynucleotide
acccctgcaggatcctttgctggtaccgcttactgggcatgcaggaatcttttcgccggttactgttagtatcgctaatcagtctagagggccccagaat

SEQ ID NO: 13 - DNA, 100 bp, Artificial Sequence
  Description of Artificial Sequence Synthetic polynucleotide
attctggggccctctagactgattagcgatactactatcctccccccttagccataaattacggagcggatgaggtaccagcaaaggatcctgcaggggt

SEQ ID NO: 14 - DNA, 100 bp, Artificial Sequence
  Description of Artificial Sequence Synthetic polynucleotide
acccctgcaggatcctttgctggtacctcatccgctccgtaatttatggctaaggggggaggatagtagtatcgctaatcagtctagagggccccagaat

SEQ ID NO: 15 - DNA, 100 bp, Artificial Sequence
  Description of Artificial Sequence Synthetic polynucleotide
attctggggccctctagactgattagcgatactctcccacctacgccttaacttttccaacatcctccatcgcggtaccagcaaaggatcctgcaggggt

SEQ ID NO: 16 - DNA, 100 bp, Artificial Sequence
  Description of Artificial Sequence Synthetic polynucleotide
acccctgcaggatcctttgctggtaccgcgatggaggatgttggaaaagttaaggcgtaggtgggagagtatcgctaatcagtctagagggccccagaat

SEQ ID NO: 17 - DNA, 100 bp, Artificial Sequence
  Description of Artificial Sequence Synthetic polynucleotide
acccctgcaggatcctttgctggtacctagaagccggccgtagaggaggaaaggatgataacttgctagtatcgctaatcagtctagatggccccagaat

SEQ ID NO: 18 - DNA, 100 bp, Artificial Sequence
  Description of Artificial Sequence Synthetic polynucleotide
attctggggccatctagactgattagcgatactagcaagttatcatcctttcctcctctacggccggcttctaggtaccagcaaaggatcctgcaggggt

SEQ ID NO: 19 - DNA, 100 bp, Artificial Sequence
  Description of Artificial Sequence Synthetic polynucleotide
acccctgcaggatcctttgctggtaccagggaaatcgtagttgattacgattatgacgtagtgtaccagtatcgctaatcagtctagagggccccagaat

SEQ ID NO: 20 - DNA, 100 bp, Artificial Sequence
  Description of Artificial Sequence Synthetic polynucleotide
attctggggccctctagactgattagcgatactggtacactacgtcataatcgtaatcaactacgatttccctggtaccagcaaaggatcctgcaggggt

SEQ ID NO: 21 - DNA, 100 bp, Artificial Sequence
  Description of Artificial Sequence Synthetic polynucleotide
acccctgcaggatcctttgctggtaccgtagactatcattttcactaacgtacagggagcggggctgagtatcgctaatcagtctagagggccccagtat

SEQ ID NO: 22 - DNA, 100 bp, Artificial Sequence
  Description of Artificial Sequence Synthetic polynucleotide
atactggggccctctagactgattagcgatactcagccccgctccctgtacgttagtgaaaatgatagtctacggtaccagcaaaggatcctgcaggggt

SEQ ID NO: 23 - DNA, 100 bp, Artificial Sequence
  Description of Artificial Sequence Synthetic polynucleotide
acccctgcaggatcctttgctggtacctaagacttggagtaagacagtgacaggtatattggttgtgagtatcgctaatcagtctagagggccccagaat

SEQ ID NO: 24 - DNA, 100 bp, Artificial Sequence
  Description of Artificial Sequence Synthetic polynucleotide
attctggggccctctagactgattagcgatactcacaaccaatatacctgtcactgtcttactccaagtcttaggtaccagcaaaggatcctgcaggggt

SEQ ID NO: 25 - DNA, 100 bp, Artificial Sequence
  Description of Artificial Sequence Synthetic polynucleotide
acccctgcaggatcctttgctggtaccnnnnnnnnnnnnnnnnnnnnnnnnnnnnnnnnnnnnnnnnagtatcgctaatcagtctagagggccccagaat

SEQ ID NO: 26 - DNA, 27 bp, Artificial Sequence
  Description of Artificial Sequence Synthetic primer
acccctgcaggatcctttgctggtacc

SEQ ID NO: 27 - DNA, 33 bp, Artificial Sequence
  Description of Artificial Sequence Synthetic primer
attctggggccctctagactgattagcgatact

SEQ ID NO: 28 - DNA, 27 bp, Artificial Sequence
  Description of Artificial Sequence Synthetic primer
acccctgcaggatcctttgctggtacc

SEQ ID NO: 29 - DNA, 33 bp, Artificial Sequence; identical to SEQ ID NO: 27
  Description of Artificial Sequence Synthetic primer

SEQ ID NO: 30 - DNA, 27 bp, Artificial Sequence
  Description of Artificial Sequence Synthetic primer
acccctgcaggatcctttgctggtacc

SEQ ID NO: 31 - DNA, 33 bp, Artificial Sequence; identical to SEQ ID NO: 27
  Description of Artificial Sequence Synthetic primer

SEQ ID NO: 32 - DNA, 19 bp, Artificial Sequence
  Description of Artificial Sequence Synthetic primer
tatttaggtgacactatag

SEQ ID NO: 33 - DNA, 20 bp, Artificial Sequence
  Description of Artificial Sequence Synthetic primer
taatacgactcactataggg

SEQ ID NO: 34 - DNA, 97 bp, Artificial Sequence
  Description of Artificial Sequence Synthetic oligonucleotide
acccctgcaggatcctttgctggtaccnnnnnnnnnnnnnnnnnnnnnnnnnnnnnnnnnnnnnnnntaagaccccgggagatctgactaatcgcta

SEQ ID NO: 35 - DNA, 99 bp, Artificial Sequence
  Description of Artificial Sequence Synthetic oligonucleotide
acccctgcaggatcctttgctggtaccagagcggaggggcgtgaggggagggagctatgagtggaaagtatcgctaatcagtctagagggccccagaat

SEQ ID NO: 36 - DNA, 99 bp, Artificial Sequence
  Description of Artificial Sequence Synthetic oligonucleotide
attctggggccctctagactgattagcgatactttccactcatagctccctcccctcacgcccctccgctctggtaccagcaaaggatcctgcaggggt

SEQ ID NO: 37 - DNA, 100 bp, Artificial Sequence
  Description of Artificial Sequence Synthetic oligonucleotide
acccctgcaggatcctttgctggtaccccgcgcgttatttccctccctcatccgttgtctcgctcagagtatcgctaatcagtctagagggccccagaat

SEQ ID NO: 38 - DNA, 100 bp, Artificial Sequence
  Description of Artificial Sequence Synthetic oligonucleotide
attctggggccctctagactgattagcgatactctgagcgagacaacggatgagggagggaaataacgcgcggggtaccagcaaaggatcctgcaggggt

SEQ ID NO: 39 - DNA, 100 bp, Artificial Sequence
  Description of Artificial Sequence Synthetic oligonucleotide
attctggggccctctagactgattagcgatactacttaacctgcacaccctcccaaatcactgctccccccccggtaccagcaaaggatcctgcaggggt

SEQ ID NO: 40 - DNA, 100 bp, Artificial Sequence
  Description of Artificial Sequence Synthetic oligonucleotide
acccctgcaggatcctttgctggtaccggggggggagcagtgatttgggagggtgtgcaggttaagtagtatcgctaatcagtctagagggccccagaat

SEQ ID NO: 41 - DNA, 100 bp, Artificial Sequence
  Description of Artificial Sequence Synthetic oligonucleotide
acccctgcaggatcctttgctggtaccgcgttatgggaaaatcaggagagagggggagggagaaagtagtatcgctaatcagtctagagggccccagaat

SEQ ID NO: 42 - DNA, 100 bp, Artificial Sequence
  Description of Artificial Sequence Synthetic oligonucleotide
attctggggccctctagactgattagcgatactactttctccctccccctctctcctgattttcccataacgcggtaccagcaaaggatcctgcaggggt

SEQ ID NO: 43 - DNA, 80 bp, Artificial Sequence
  Description of Artificial Sequence Synthetic oligonucleotide
tgtaatacgactcactatagggcgaattgggcccgacgtcgcatgctcccggccgccatggcggccgcgggaattcgatt

SEQ ID NO: 44 - DNA, 98 bp, Artificial Sequence
  Description of Artificial Sequence Synthetic oligonucleotide
atcactagtgaattcgcggccgcctgcaggtcgaccatatgggagagctcccaacgcgttggatgcatagcttgagtattctatagtgtcacctaaat

SEQ ID NO: 45 - DNA, 79 bp, Artificial Sequence
  Description of Artificial Sequence Synthetic oligonucleotide
atcgaattcccgcggccgccatggcggccgggagcatgcgacgtcgggcccaattcgccctatagtgagtcgtattaca

SEQ ID NO: 46 - DNA, 99 bp, Artificial Sequence
  Description of Artificial Sequence Synthetic oligonucleotide
atttaggtgacactatagaatactcaagctatgcatccaacgcgttgggagctctcccatatggtcgacctgcaggcggccgcgaattcactagtgatt

SEQ ID NO: 47 - DNA, 58 bp, Artificial Sequence
  Description of Artificial Sequence Synthetic oligonucleotide
cctgcacaccctcccaaatcactgctccccccccggtaccagcaaaggatcctgcagg

SEQ ID NO: 48 - DNA, 44 bp, Artificial Sequence
  Description of Artificial Sequence Synthetic oligonucleotide
gggggagggagaaagtagtatcgctaatcagtctagagggcccc

SEQ ID NO: 49 - DNA, 40 bp, Artificial Sequence
  Description of Artificial Sequence Synthetic oligonucleotide
ggggccctctagactgattagcgatactactatcctcccc

SEQ ID NO: 50 - DNA, 54 bp, Artificial Sequence
  Description of Artificial Sequence Synthetic oligonucleotide
cttaacttttccaacatcctccatcgcggtaccagcaaaggatcctgcaggggt

SEQ ID NO: 51 - DNA, 57 bp, Artificial Sequence
  Description of Artificial Sequence Synthetic oligonucleotide
acccctgcaggatgataacttgctagtatcgctaatcagtctagatggccccagaat

SEQ ID NO: 52 - DNA, 67 bp, Artificial Sequence
  Description of Artificial Sequence Synthetic oligonucleotide
acccctgcaggatcctttgctggtaccgtagactatcattttcactaacgtacagggagcgcagtat

SEQ ID NO: 53 - DNA, 54 bp, Artificial Sequence
  Description of Artificial Sequence Synthetic oligonucleotide
aagacttggagtaagacagtgacaggtatattggttgtgagtatcgctaatcag